SEQUENCE LISTING (11 sequences)

SEQ ID NO: 1 - protein, 10 aa, Artificial Sequence
  Description of Artificial Sequence Synthetic peptide
GFSFSSTYVC

SEQ ID NO: 2 - protein, 16 aa, Artificial Sequence
  Description of Artificial Sequence Synthetic peptide
CIYTGDGTNYSASWAK

SEQ ID NO: 3 - protein, 11 aa, Artificial Sequence
  Description of Artificial Sequence Synthetic peptide
PDITYGFAINF

SEQ ID NO: 4 - protein, 11 aa, Artificial Sequence
  Description of Artificial Sequence Synthetic peptide
QASQSISSRLA

SEQ ID NO: 5 - protein, 7 aa, Artificial Sequence
  Description of Artificial Sequence Synthetic peptide
RASTLAS

SEQ ID NO: 6 - protein, 10 aa, Artificial Sequence
  Description of Artificial Sequence Synthetic peptide
QCTGYGISWP

SEQ ID NO: 7 - protein, 120 aa, Artificial Sequence
  Description of Artificial Sequence Synthetic polypeptide
QVQLVESGGGVVQPGRSLRLSCAASGFSFSSTYVCWVRQAPGKGLEWIACIYTGDGTNYSASWAKGRFTISKDSSKNTVYLQMNSLRAEDTAVYFCARPDITYGFAINFWGPGTLVTVSS

SEQ ID NO: 8 - protein, 130 aa, Artificial Sequence
  Description of Artificial Sequence Synthetic polypeptide
MDMRVPAQLLGLLLLWLRGARCDIQMTQSPSSLSASVGDRVTIKCQASQSISSRLAWYQQKPGKPPKLLIYRASTLASGVPSRFSGSGSGTDFTLTISSLQPEDVATYYCQCTGYGISWPIGGGTKVEIK

SEQ ID NO: 9 - protein, 137 aa, Artificial Sequence
  Description of Artificial Sequence Synthetic polypeptide
METGLRGLLLVAVLKGVQCQSLEESGGDLVKPGASLTLTCTASGFSFSSTYVCWVRQAPGKGLEWIACIYTGDGTNYSASWAKGRFTISKPSSTTVTLQMTSLTPADTATYFCARPDITYGFAINFWGPGTLVTVSS

SEQ ID NO: 10 - protein, 131 aa, Artificial Sequence
  Description of Artificial Sequence Synthetic polypeptide
MDTRAPTQLLGLLLLWLPGARSADIVMTQTPSSASEPVGGTVTIKCQASQSISSRLAWYQQKPGQPPKLLIYRASTLASGVPSRFKGSGSGTEFTLTISDLECADAATYYCQCTGYGISWPIGGGTEVVVK

SEQ ID NO: 11 - protein, 6 aa, Artificial Sequence
  Description of Artificial Sequence Synthetic 6xHis tag
HHHHHH

(19) United States
(12) Patent Application Publication
(10) Pub. No.: US 2021/0386856 A1
(43) Pub. Date: Dec. 16, 2021

(54) COMBINATION THERAPY WITH NEOANTIGEN VACCINE

(71) Applicant: BIONTECH US INC., Cambridge, MA (US)

(72) Inventors: Robert Ang, Waban, MA (US); Richard Gaynor, Carmel, IN (US); Matthew Goldstein, Jamaica Plain, MA (US)

(21) Appl. No.: 17/251,285

(22) PCT Filed: Jun. 11, 2019
(86) PCT No.: PCT/US2019/036632

§ 371 Date: Dec. 11, 2020

Related U.S. Application Data
(60) Provisional application No. 62/684,013, filed on Jun. 12, 2018.

(51) Int. Cl. A61K 39/395; A61K 39/00; A61K 39/39; A61P 35/00

(52) U.S. Cl. CPC A61K 39/3955; A61K 39/0011; A61K 39/39; A61P 35/00; A61K 2039/55; A61K 2039/5158; A61K 2039/55583; A61K 2039/545; A61K 2039/5154

(57) ABSTRACT

The present invention relates to neoplasia vaccine or immunogenic composition administered in combination with other agents, such as checkpoint blockade inhibitors for the treatment or prevention of neoplasia in a subject

### CROSS-REFERENCE

This application claims priority to U.S. Provisional Application No. 62/684,013, filed on Jun. 12, 2018; which is incorporated herein by reference in its entirety.

### BACKGROUND

Cancer immunotherapy is the use of the immune system to treat cancer. Immunotherapies exploit the fact that cancer cells often have molecules on their surface that can be detected by the immune system, known as tumor antigens, which are often proteins or other macromolecules (e.g. carbohydrates). Active immunotherapy directs the immune system to attack tumor cells by targeting tumor antigens. Passive immunotherapies enhance existing anti-tumor responses and include the use of monoclonal antibodies, lymphocytes and cytokines. Tumor vaccines are typically composed of tumor antigens and immunostimulatory molecules (e.g., adjuvants, cytokines or TLR ligands) that work together to induce antigen-specific cytotoxic T cells (CTLs) that recognize and lyse tumor cells. One of the critical barriers to developing curative and tumor-specific immunotherapy is the identification and selection of highly specific and restricted tumor antigens to avoid autoimmunity.

Tumor neoantigens, which arise as a result of genetic change (e.g., inversions, translocations, deletions, missense mutations, splice site mutations, etc.) within malignant cells, represent the most tumor-specific class of antigens and can be patient-specific or shared. Tumor neoantigens are unique to the tumor cell as the mutation and its corresponding protein are present only in the tumor. They also avoid central tolerance and are therefore more likely to be immunogenic. Therefore, tumor neoantigens provide an excellent target for immune recognition including by both humoral and cellular immunity. Accordingly, there is still a need for developing additional cancer therapeutics.

### SUMMARY

In some aspects, provided herein is a method of treating or preventing a neoplasia in a human subject in need thereof comprising administering to a subject in need thereof: a first component comprising (i) a peptide comprising a neoepitope of a protein, (ii) a polynucleotide encoding the peptide, (iii) one or more APCs comprising the peptide or the polynucleotide encoding the peptide or (iv) a T cell receptor (TCR) specific for the neoepitope in complex with an HLA protein; and a second component comprising at least two inhibitors, wherein the at least two inhibitors comprise: nivolumab and an anti-CD40 agonist antibody, or nivolumab and ipilimumab, or ipilimumab and an anti-CD40 agonist antibody.

In some embodiments, the anti-CD40 agonist antibody comprises a heavy chain complement determining region 1 (HCDR1) of SEQ ID NO: 1 a HCDR2 of SEQ ID NO: 2, and a HCDR3 of SEQ ID NO: 3, a light chain CDR1 (LCDR1) of SEQ ID NO: 4, a LCDR2 of SEQ ID NO: 5 and an LCDR3 of SEQ ID NO: 6. In some embodiments, the anti-CD40 agonist antibody comprises a heavy chain variable sequence (V_{H}) with at least 80% sequence identity to SEQ ID NO: 7, and/or a light chain variable sequence (V_{L}) with at least 80% sequence identity to SEQ ID NO: 8. In some embodiments, the anti-CD40 agonist antibody comprises a heavy chain sequence with at least 70% sequence identity to SEQ ID NO: 9, and/or a light chain sequence with at least 70% sequence identity to SEQ ID NO: 10. In some embodiments, the anti-CD40 agonist antibody is a human or humanized antibody. In some embodiments, the anti-CD40 agonist antibody is APX005M.

In some embodiments, the anti-CD40 agonist antibody is an antibody other than APX005M. In some embodiments, the anti-CD40 agonist antibody is ABBV-927. In some embodiments, the anti-CD40 agonist antibody is CDX-1140. In some embodiments, the anti-CD40 agonist antibody is ADC-1013.

In some embodiments, the first component comprises a neoplasia vaccine or immunogenic composition.

In some embodiments, the first component further comprises an adjuvant. In some embodiments, the adjuvant is poly-ICLC.

In some embodiments, the peptide comprises at least two, at least three, at least four or at least five peptides. In some embodiments, the peptide comprises at most 15, at most 20, at most 25 or at most 30 peptides. In some embodiments, the peptide is from 5 to 50 amino acids in length. In some embodiments, the peptide is from 14 to 35 amino acids in length. In some embodiments, the neoepitope of each peptide is unique.

In some embodiments, the first component further comprises a pH modifier. In some embodiments, the first component further comprises a pharmaceutically acceptable carrier.

In some embodiments, the subject is suffering from a neoplasia selected from the group consisting of Non-Hodgkin's Lymphoma (NHL), clear cell Renal Cell Carcinoma (ccRCC), melanoma, sarcoma, leukemia or a cancer of the bladder, colon, brain, breast, head and neck, endometrium, lung, ovary, pancreas or prostate. In some embodiments, the neoplasia is metastatic melanoma. In some embodiments, the subject has no detectable neoplasia but is at high risk for disease recurrence. In embodiments, the cancer is selected from the group consisting of: adrenal, bladder, breast, cervical, colorectal, glioblastoma, head and neck, kidney chromophobe, kidney clear cell, kidney papillary, liver, lung adenocarcinoma, lung squamous, ovarian, pancreatic, melanoma, stomach, uterine corpus endometrial, and uterine carcinosarcoma. In embodiments, the cancer is selected from the group consisting of: prostate cancer, bladder, lung squamous, NSCLC, breast, head and neck, lung adenocarcinoma, GBM, Glioma, CML, AML, supretentorial ependyomas, acute promyelocytic leukemia, solitary fibrous tumors, and crizotinib resistant cancer. In embodiments, the cancer is selected from the group consisting of: CRC, head and neck, stomach, lung squamous, lung adenocarcinoma, prostate, bladder, stomach, renal cell carcinoma, and uterine. In embodiments, the cancer is selected from the group consisting of: melanoma, lung squamous, DLBCL, uterine, head and neck, uterine, liver, and CRC. In embodiments, the cancer is selected from the group consisting of: lymphoid cancer; Burkitt lymphoma, neuroblastoma, prostate adenocarcinoma, colorectal adenocarcinoma; Uterine/Endometrium Adenocarcinoma; MSI+; endometrium serous carcinoma; endometrium carcinosarcoma-malignant mesodermal mixed tumour; glioma; astrocytoma; GBM, acute myeloid leukemia associated with MDS; chronic lymphocytic leukemia-small lymphocytic lymphoma; myelodysplastic syndrome; acute myeloid leukemia; luminal NS carcinoma of breast; chronic myeloid leukemia; ductal carcinoma of pancreas; chronic myelomonocytic leukemia; myelofibrosis; myelodysplastic syndrome; prostate adenocarcinoma; essential thrombocythaemia; and medullomyoblastoma. In embodiments, the cancer is selected from the group consisting of: colorectal, uterine, endometrial, and stomach. In embodiments, the cancer is selected from the group consisting of: cervical, head and neck, anal, stomach, Burkitt's lymphoma, and nasopharyngeal carcinoma. In embodiments, the cancer is selected from the group consisting of: bladder, colorectal, and stomach. In embodiments, the cancer is selected from the group consisting of: lung, CRC, melanoma, breast, NSCLC, and CLL. In embodiments, the subject is a partial or non-responder to checkpoint inhibitor therapy. In embodiments, the cancer is selected from the group consisting of: bladder urothelial carcinoma (BLCA), breast invasive carcinoma (BRCA), breast cancer, cervical squamous cell carcinoma and endocervical adenocarcinoma (CESC), chronic lymphocytic leukemia (CLL), colorectal cancer (CRC), glioblastoma multiforme (GBM), head and neck squamous cell carcinoma (HNSC), kidney renal papillary cell carcinoma (KIRP), liver hepatocellular carcinoma (LIHC), lung adenocarcinoma (LUAD), lung squamous cell carcinoma (LUSC), pancreatic adenocarcinoma (PAAD), Prostate Cancer, skin cutaneous melanoma (SKCM), stomach adenocarcinoma (STAD), thyroid adenocarcinoma (THCA), and uterine corpus endometrioid carcinoma (UCEC). In embodiments, the cancer is selected from the group consisting of: colorectal cancer, uterine cancer, endometrium cancer, stomach cancer, and Lynch syndrome. In embodiments, the cancer is an MSI+ cancer.

In some embodiments, the first component is administered before the second component. In some embodiments, the second component is administered before the first component. In some embodiments, the first component is administered on the same day as the second component. In some embodiments, the first component is administered before the second component. In some embodiments, administration of nivolumab is initiated before initiation of administration of the first component. In some embodiments, administration of nivolumab is initiated before initiation of administration of the anti-CD40 agonist antibody. In some embodiments, administration of nivolumab is initiated before initiation of administration of ipilimumab. In some embodiments, administration of ipilimumab is on the same day as the initial administration of the first component. In some embodiments, administration of APX005M is initiated on the same day as the initial administration of the first component. In some embodiments, administration of nivolumab continues every 12-36 or more weeks after a first administration of nivolumab. In some embodiments, administration of nivolumab continues every 2, 3, 4, 6 or 8 weeks after the first administration of nivolumab. In some embodiments, administration of an inhibitor, such as a checkpoint inhibitor or CD40 agonist, is initiated following tumor resection. In some embodiments, administration of the first component is in a prime boost dosing regimen.

In some embodiments, administration of the first component is at weeks 1, 2, 3 or 4 as a prime. In some embodiments, administration of the first component is at months 2, 3, 4 or 5 as a boost. In some embodiments, administration of the first component is at weeks 19, 20, 21, 22, 23 or 24 as a boost.

In some embodiments, the peptide is administered at an average dose level of about 300-500 μg/ml per peptide. In some embodiments, a total dose of the peptide administered is from 4-8 mg. In some embodiments, nivolumab is administered at a dose of from 200-260 mg. In some embodiments, APX005M is administered at a dose of from 0.05-0.2 mg/kg. In some embodiments, APX005M is administered at a dose of from 0.5-2.0 mg/kg.

In some embodiments, the first component and/or the second component is administered intravenously or subcutaneously.

In some embodiments, a dose of the peptide is divided into at least 2, at least 3, at least 4 or at least 5 sub-doses. In some embodiments, each sub-dose of the peptide comprises at least 4 or at least 5 peptides.

In some embodiments, each peptide is administered at a dose of from 200-400 μg. In some embodiments, each sub-dose is administered at a different location of the subject.

In some embodiments, the method further comprises administering of one or more additional agents. In some embodiments, the additional agents are selected from the group consisting of: chemotherapeutic agents, anti-angiogenesis agents and agents that reduce immune-suppression.

In some embodiments, administration of ipilimumab is in a prime boost regimen. In some embodiments, administration of ipilimumab is at day 1, 2, 3 or 4 as a prime. In some embodiments, administration of ipilimumab is at months 2 or 3 as a boost. In some embodiments, administration of APX005M is in a prime boost regimen. In some embodiments, administration of APX005M is at week 1, 2, 3, 4 or 5 as a prime. In some embodiments, administration of APX005M is at months 2 or 3 as a boost.

In some aspects, provided herein is a method of treating or preventing metastatic melanoma in a human subject in need thereof that has been treated with nivolumab comprising administering to the subject: at least five peptides each comprising a unique neoepitope of a protein at a dose of from 200-400 μg of each peptide; and then an anti-CD40 agonist antibody that is APX005M at a dose of from 0.05-2.0 mg/kg.

In some aspects, provided herein is a method of treating or preventing metastatic melanoma in a human subject in need thereof that has been treated with nivolumab comprising administering to the subject: at least five peptides each comprising a unique neoepitope of a protein at a dose of from 200-400 μg of each peptide; and then ipilimumab at a dose of from 0.5-1.5 mg/kg.

In some aspects, provided herein is a method of treating or preventing cancer in a human subject in need thereof that has been treated with nivolumab comprising administering to the subject an anti-CD40 agonist antibody at a dose of less than 1.0 mg/kg or at a dose of less than 0.1 mg/kg.

In some aspects, provided herein is a method of treating or preventing cancer in a human subject in need thereof that has been treated with nivolumab comprising administering to the subject an anti-CD40 agonist antibody at a dose of from 1-95% a dosage of the anti-CD40 agonist antibody normally administered in a monotherapy regimen. In some embodiments the anti-CD40 agonist antibody is APX005M.

In some aspects, provided herein is a method of treating or preventing cancer in a human subject in need thereof that has been treated with nivolumab at a dose of from 1-95% a dosage of the nivolumab normally administered in a monotherapy regimen, the method comprising administering to the subject an anti-CD40 agonist antibody at a dose of less than 1.0 mg/kg or at a dose of less than 0.1 mg/kg. In some embodiments the anti-CD40 agonist antibody is APX005M.

In some aspects, provided herein is a method of treating or preventing cancer in a human subject in need thereof that has been treated with nivolumab at a dose of from 1-95% a dosage of the nivolumab normally administered in a monotherapy regimen, the method comprising administering to the subject an anti-CD40 agonist antibody at a dose of from 1-95% a dosage of the anti-CD40 agonist antibody normally administered in a monotherapy regimen. In some embodiments the anti-CD40 agonist antibody is APX005M.

In some aspects, provided herein is a method of treating or preventing cancer in a human subject in need thereof comprising administering to the subject: nivolumab at a dose of less than 1.0 mg/kg or at a dose of less than 3.0 mg/kg or at a dose of from 1-95% a dosage of the nivolumab normally administered in a monotherapy regimen; and an anti-CD40 agonist antibody at a dose of less than 1.0 mg/kg or at a dose of less than 0.1 mg/kg or at a dose of from 1-95% a dosage of the anti-CD40 agonist antibody normally administered in a monotherapy regimen. In some embodiments the anti-CD40 agonist antibody is APX005M.

In some aspects, provided herein is a method of treating or preventing cancer in a human subject in need thereof that has been treated with nivolumab comprising administering to the subject ipilimumab at a dose of less than 1.0 mg/kg.

In some aspects, provided herein is a method of treating or preventing cancer in a human subject in need thereof that has been treated with nivolumab comprising administering to the subject ipilimumab at a dose of from 1-95% a dosage of the ipilimumab normally administered in a monotherapy regimen.

In some aspects, provided herein is a method of treating or preventing cancer in a human subject in need thereof that has been treated with nivolumab at a dose of from 1-95% a dosage of the nivolumab normally administered in a monotherapy regimen, the method comprising administering to the subject ipilimumab at a dose of less than 1.0 mg/kg.

In some aspects, provided herein is a method of treating or preventing cancer in a human subject in need thereof that has been treated with nivolumab at a dose of from 1-95% a dosage of the nivolumab normally administered in a monotherapy regimen, the method comprising administering to the subject ipilimumab at a dose of from 1-95% a dosage of the ipilimumab normally administered in a monotherapy regimen.

In some aspects, provided herein is a method of treating or preventing cancer in a human subject in need thereof that has been treated with nivolumab comprising administering to the subject: nivolumab at a dose of less than 1.0 mg/kg or at a dose of less than 3.0 mg/kg or at a dose of from 1-95% a dosage of the nivolumab normally administered in a monotherapy regimen; and ipilimumab at a dose of less than 1.0 mg/kg or at a dose of from 1-95% a dosage of the ipilimumab normally administered in a monotherapy regimen.

In some embodiments, the method further comprises administering to the subject at least five peptides each comprising a unique neoepitope of a protein at a dose of from 100-500 μg of each peptide.

In some aspects, provided herein is a composition comprising: a first component comprising (i) a peptide comprising a neoepitope of a protein, (ii) a polynucleotide encoding the peptide, (iii) one or more APCs comprising the peptide or the polynucleotide encoding the peptide or (iv) a T cell receptor (TCR) specific for the neoepitope in complex with an HLA protein; and a second component comprising at least two inhibitors, wherein the at least two inhibitors comprise: nivolumab and an anti-CD40 agonist antibody, or nivolumab and ipilimumab, or ipilimumab and an anti-CD40 agonist antibody.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 depicts an example of a study schematic described herein.

FIG. 2A depicts an example of a dosing schedule described herein.

FIG. 2B depicts an example of a dosing schedule described herein.

FIG. 2C depicts an example of a dosing schedule described herein.

### DETAILED DESCRIPTION

Described herein are new immunotherapeutic agents and uses thereof based on the discovery of neoantigens arising from mutational events unique to an individual's tumor. Accordingly, the present disclosure described herein provides peptides, polynucleotides encoding the peptides, and peptide binding agents that can be used, for example, to stimulate an immune response to a tumor associated antigen or neoepitope, to create an immunogenic composition or cancer vaccine for use in treating disease.

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

### I. Definitions

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

“Major Histocompatibility Complex” or “MHC” is a cluster of genes that plays a role in control of the cellular interactions responsible for physiologic immune responses. In humans, the MHC complex is also known as the human leukocyte antigen (HLA) complex. For a detailed description of the MHC and HLA complexes, see, Paul, Fundamental Immunology, 3^{rd} Ed., Raven Press, New York (1993). “Proteins or molecules of the major histocompatibility complex (MHC)”, “MHC molecules”, “MHC proteins” or “HLA proteins” are to be understood as meaning proteins capable of binding peptides resulting from the proteolytic cleavage of protein antigens and representing potential lymphocyte epitopes, (e.g., T cell epitope and B cell epitope) transporting them to the cell surface and presenting them there to specific cells, in particular cytotoxic T-lymphocytes, T-helper cells, or B cells. The major histocompatibility complex in the genome comprises the genetic region whose gene products expressed on the cell surface are important for binding and presenting endogenous and/or foreign antigens and thus for regulating immunological processes. The major histocompatibility complex is classified into two gene groups coding for different proteins, namely molecules of MHC class I and molecules of MHC class II. The cellular biology and the expression patterns of the two MHC classes are adapted to these different roles.

“Human Leukocyte Antigen” or “HLA” is a human class I or class II Major Histocompatibility Complex (MHC) protein (see, e.g., Stites, et al., Immunology, 8^{th} Ed., Lange Publishing, Los Altos, Calif. (1994).

“Polypeptide”, “peptide” and their grammatical equivalents as used herein refer to a polymer of amino acid residues. A “mature protein” is a protein which is full-length and which, optionally, includes glycosylation or other modifications typical for the protein in a given cellular environment. Polypeptides and proteins disclosed herein (including functional portions and functional variants thereof) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetyl aminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine. The present disclosure further contemplates that expression of polypeptides described herein in an engineered cell can be associated with post-translational modifications of one or more amino acids of the polypeptide constructs. Non-limiting examples of post-translational modifications include phosphorylation, acylation including acetylation and formylation, glycosylation (including N-linked and O-linked), amidation, hydroxylation, alkylation including methylation and ethylation, ubiquitination, addition of pyrrolidone carboxylic acid, formation of disulfide bridges, sulfation, myristoylation, palmitoylation, isoprenylation, farnesylation, geranylation, glypiation, lipoylation and iodination. The term “polypeptide” or “peptide” may also mean that a polypeptide that has been separated from components that naturally accompany it. Typically, the polypeptide is isolated when it is at least 60%, by weight, free from the proteins and naturally-occurring organic molecules with which it is naturally associated. In some embodiments, the preparation is at least 75%, at least 90%, or at least 99%, by weight, a polypeptide. An isolated polypeptide may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

An “immunogenic” peptide or an “immunogenic” epitope or “peptide epitope” is a peptide that comprises an allele-specific motif such that the peptide will bind an HLA molecule and induce a cell-mediated or humoral response, for example, cytotoxic T lymphocyte (CTL (e.g., CD8⁺)), helper T lymphocyte (Th (e.g., CD4⁺)) and/or B lymphocyte response. Thus, immunogenic peptides described herein are capable of binding to an appropriate HLA molecule and thereafter inducing a CTL (cytotoxic) response, or a HTL (and humoral) response, to the peptide.

The term “neoantigen” or “neoantigenic” means a class of tumor antigens that arises from a tumor-specific mutation(s) which alters the amino acid sequence of genome encoded proteins. Neoantigens encompass, but are not limited to, tumor antigens which arise from, for example, substitution in the protein sequence, frame shift mutation, fusion polypeptide, in-frame deletion, insertion, expression of endogenous retroviral polypeptides, and tumor-specific overexpression of polypeptides.

The term “neoantigen peptide” and “neoantigenic peptide”, used interchangeably with “peptide” in the present specification, refers to a series of residues, typically L-amino acids, connected one to the other, typically by peptide bonds between the α-amino and carboxyl groups of adjacent amino acids. Similarly, the term “polypeptide” is used interchangeably with “mutant polypeptide”, “neoantigen polypeptide” and “neoantigenic polypeptide” in the present specification to designate a series of residues, e.g., L-amino acids, connected one to the other, typically by peptide bonds between the α-amino and carboxyl groups of adjacent amino acids. The polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described. A peptide or polypeptide as used herein comprises at least one flanking sequence. The term “flanking sequence” as used herein refers to a fragment or region of the neoantigen peptide that is not a part of the neoepitope. The term “residue” refers to an amino acid residue or amino acid mimetic residue incorporated into a peptide or protein by an amide bond or amide bond mimetic, or nucleic acid (DNA or RNA) that encodes the amino acid or amino acid mimetic.

By “neoplasia” is meant any disease that is caused by or results in inappropriately high levels of cell division, inappropriately low levels of apoptosis, or both. For example, cancer is an example of a neoplasia. Examples of cancers include, without limitation, leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma). Lymphoproliferative disorders are also considered to be proliferative diseases.

The term “neoplasia vaccine” is meant to refer to a pooled sample of neoplasia/tumor specific neoantigens, for example at least two, at least three, at least four, at least five, or more neoantigenic peptides. A “vaccine” is to be understood as meaning a composition for generating immunity for the prophylaxis and/or treatment of diseases (e.g., neoplasia/tumor). Accordingly, vaccines are medicaments which comprise antigens and are intended to be used in humans or animals for generating specific defense and protective substance by vaccination. A “vaccine composition” or a “neoplasia vaccine composition” can include a pharmaceutically acceptable excipient, earner or diluent.

Immune checkpoints are inhibitory pathways that slow down or stop immune reactions and prevent excessive tissue damage from uncontrolled activity of immune cells. By “checkpoint inhibitor” is meant to refer to any small molecule chemical compound, antibody, nucleic acid molecule, or polypeptide, or fragments thereof, which inhibit the inhibitory pathways, allowing more extensive immune activity. In certain embodiments, the checkpoint inhibitor is an inhibitor of the programmed death-1 (PD-1) pathway, for example an anti-PD1 antibody, such as, but not limited to Nivolumab. In other embodiments, the checkpoint inhibitor is an anti-cytotoxic T-lymphocyte-associated antigen (CTLA-4) antibody. In additional embodiments, the checkpoint inhibitor is targeted at another member of the CD28CTLA4 (g superfamily such as BTLA, LAG3, ICOS, PDL1 or KIR Page et al., Annual Review of Medicine 65:27 (2014)). In some cases targeting a checkpoint inhibitor is accomplished with an inhibitory antibody or similar molecule. In other cases, it is accomplished with an agonist for the target.

In further additional embodiments, the inhibitor is targeted at a member of the TNF Superfamily such as CD40, OX40, CD 137, GITR, CD27 or TIM-3. In some cases targeting a member of the TNF Superfamily is accomplished with an inhibitory antibody or similar molecule. In other cases, it is accomplished with an agonist for the target; examples of this class include the stimulatory targets CD40, OX40 and GITR.

The term “combination” embraces the administration of a vaccine or immunogenic composition (e.g. a pooled sample of neoplasia/tumor specific neo antigens) and one or more inhibitors, such as a checkpoint inhibitor or CD40 agonist, as part of a treatment regimen intended to provide a beneficial (additive or synergistic) effect from the co-action of one or more of these therapeutic agents. The combination may also include one or more additional agents, for example, but not limited to, chemotherapeutic agents, anti-angiogenesis agents and agents that reduce immune-suppression. The beneficial effect of the combination includes, but is not limited to, pharmacokinetic or pharmacodynamic co-action resulting from the combination of therapeutic agents. Administration of these therapeutic agents in combination typically is carried out over a defined time period (for example, minutes, hours, days, or weeks depending upon the combination selected).

“Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. For example, one combination of the present invention may comprise a pooled sample of tumor specific neoantigens and an inhibitor, such as a checkpoint inhibitor or CD40 agonist, administered at the same or different times, or the composition can be formulated as a single, co-formulated pharmaceutical composition comprising the two compounds. As another example, a combination of the present invention (e.g., a pooled sample of tumor specific neoantigens and an inhibitor, such as a checkpoint inhibitor (e.g., an anti-CTLA4 antibody), and/or CD40 agonist) may be formulated as separate pharmaceutical compositions that can be administered at the same or different time. As used herein, the term “simultaneously” is meant to refer to administration of one or more agents at the same time. For example, in certain embodiments, a vaccine or immunogenic composition and an inhibitor, such as a checkpoint inhibitor or CD40 agonist, are administered simultaneously. Simultaneously includes administration contemporaneously, that is during the same period of time. In certain embodiments, the one or more agents are administered simultaneously in the same hour, or simultaneously in the same day. Sequential or substantially simultaneous administration of each therapeutic agent can be effected by any appropriate route including, but not limited to, oral routes, intravenous routes, sub-cutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by intravenous injection while the other component(s) of the combination may be administered orally. The components may be administered in any therapeutically effective sequence. The phrase “combination” embraces groups of compounds or non-drug therapies useful as part of a combination therapy.

The term “pharmaceutically acceptable” refers to approved or approvable by a regulatory agency of the Federal or a state government or listed in the U.S. Pharmacopeia or other generally recognized pharmacopeia for use in animals, including humans. Examples of cancers include, without limitation, leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma). Lymphoproliferative disorders are also considered to be proliferative diseases.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” of pooled tumor specific neoantigens as recited herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled tumor specific neoantigens provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the terms “prevent,” “preventing,” “prevention,” “prophylactic treatment,” and the like, refer to reducing the probability of developing a disease or condition in a subject, who does not have, but is at risk of or susceptible to developing a disease or condition.

The term “prime/boost” or “prime/boost dosing regimen” is meant to refer to the successive administrations of a vaccine or immunogenic or immunological compositions. The priming administration (priming) is the administration of a first vaccine or immunogenic or immunological composition type and may comprise one, two or more administrations. The boost administration is the second administration of a vaccine or immunogenic or immunological composition type and may comprise one, two or more administrations, and, for instance, may comprise or consist essentially of annual administrations. In certain embodiments, administration of the neoplasia vaccine or immunogenic composition is in a prime/boost dosing regimen.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

A “receptor” is to be understood as meaning a biological molecule or a molecule grouping capable of binding a ligand. A receptor may serve, to transmit information in a cell, a cell formation or an organism. The receptor comprises at least one receptor unit and frequently contains two or more receptor units, where each receptor unit may consist of a protein molecule, in particular a glycoprotein molecule. The receptor has a structure that complements the structure of a ligand and may complex the ligand as a binding partner. Signaling information may be transmitted by conformational changes of the receptor following binding with the ligand on the surface of a cell. According to the invention, a receptor may refer to particular proteins of MHC classes I and II capable of forming a receptor/ligand complex with a ligand, in particular a peptide or peptide fragment of suitable length.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The terms “treat,” “treated,” “treating,” “treatment,” and the like are meant to refer to reducing or ameliorating a disorder and/or symptoms associated therewith (e.g., a neoplasia or tumor). “Treating” may refer to administration of the combination therapy to a subject after the onset, or suspected onset, of a cancer. “Treating” includes the concepts of “alleviating”, which refers to lessening the frequency of occurrence or recurrence, or the severity, of any symptoms or other ill effects related to a cancer and/or the side effects associated with cancer therapy. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

The term “therapeutic effect” refers to some extent of relief of one or more of the symptoms of a disorder (e.g., a neoplasia or tumor) or its associated pathology. “Therapeutically effective amount” as used herein refers to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the compounds of the invention employed in a pharmaceutical composition at levels lower than that required in order to achieve the desired therapeutic effect and gradually increase the dosage until the desired effect is achieved.

An “adverse reaction” or AE may generally refer to any untoward medical occurrence in a patient administered a pharmaceutical product, which may not necessarily have a causal relationship with the treatment. An AE can be any unfavorable and unintended sign (e.g., including an abnormal laboratory finding), symptom, or disease temporally associated with the use of the investigational product, whether or not it is considered to be study treatment related. This includes any newly occurring event or previous condition that has increased in severity or frequency since the administration of study treatment. Abnormal laboratory values or test results constitute AEs only if they induce clinical signs or symptoms, are considered clinically significant, or require therapy.

Progression of the cancer under study is not considered an AE unless it is considered to be drug-related by the patients' care team. An “adverse drug reaction (ADR)” is defined as all noxious and unintended responses to a medicinal product, related to any dose. A causal relationship between the medicinal product and an AE is at least a reasonable possibility—i.e., the relationship cannot be ruled out. An expected AE is one that is listed or characterized in the applicable product information, e.g., the current D3. An unexpected AE is one that is not identified in nature, severity, or frequency as described in the applicable product information, e.g., the current D3. An unexpected ADR is defined as an ADR where the nature or severity is not consistent with the applicable product information. ADRs that are more specific or more severe than described in the IB(s) can also be considered unexpected. A “serious adverse event” or SAE is any AE, occurring at any dose and regardless of causality that: results in death; is life-threatening (Life-threatening means that the patient was at immediate risk of death from the reaction as it occurred, i.e., it does not include a reaction which hypothetically might have caused death had it occurred in a more severe form.); requires in-patient hospitalization or prolongation of existing hospitalization (hospitalization admissions and/or surgical operations scheduled to occur during the study period but planned prior to study entry are not considered AEs if the illness or disease existed before the patient was enrolled in the study, provided that it did not deteriorate in an unexpected manner during the study (e.g., surgery performed earlier than planned)); results in persistent or significant disability/incapacity (disability is defined as a substantial disruption of a person's ability to conduct normal life functions.); is a congenital anomaly/birth defect or is an important medical event, defined as an event that may not result in death, be life-threatening, or require hospitalization but may be considered an SAE when, based on appropriate medical judgment, it may jeopardize the patient or patients and may require medical or surgical intervention to prevent one of the outcomes listed in the definitions for SAEs. Examples of such medical events include allergic bronchospasm requiring intensive treatment in an emergency room or at home, blood dyscrasias or convulsions that do not result in in-patient hospitalization, or the development of drug dependency or drug abuse.

Each patient can be carefully monitored for the development of any AEs from the signing of consent through 30 days following the cessation of treatment. This information can be obtained in the form of non-leading questions (e.g., “How are you feeling?”) and from signs and symptoms detected during each examination, observations of study personnel, and spontaneous reports from patients. All AEs (serious and non-serious) spontaneously reported by the patient and/or in response to an open question from study personnel or revealed by observation, physical examination, or other diagnostic procedures may be recorded on the appropriate page of the eCRF. When possible, signs and symptoms indicating a common underlying pathology can be noted as one comprehensive event. All SAEs that occur from the signing of the ICF through 90 days after the last dose of nivolumab, or 30 days after the last dose of nivolumab if the patient initiates new anticancer therapy, should be reported by the Investigator to the Sponsor assigned to the study (see below) within 1 working day from the point in time when the Investigator becomes aware of the SAE. Should the Sponsor not be available for any reason, there may be an alternate physician contact. All SAEs may be reported whether or not considered causally related to the study treatment. SAE forms may be completed, and the information collected may include patient number, a narrative description of the event, and an assessment by the Investigator as to the severity of the event and relatedness to study treatment. A sample of the follow-up information on the SAE may be requested by the Sponsor or CRO.

The present invention relates to methods for the treatment of neoplasia, and more particularly tumors, by administering to a subject a neoplasia vaccine or immunogenic composition comprising a plurality of neoplasia/tumor specific neoantigens and at least one an inhibitor, such as a checkpoint inhibitor or CD40 agonist.

Human tumors contain large numbers of unique deoxyribonucleic acid (DNA) mutations that result in altered amino acid sequences of the encoded proteins. These novel protein sequences, known as neoantigens, range from single amino acid changes (caused by missense mutations) to the addition of long regions of novel amino acid sequences due to frame shifts, read-through of termination codons, or translation of intron regions (novel open reading frames [neoORFs]). Tumor neoantigens arise mostly because of mutations in tumors. Therefore, they are extremely tumor-specific and are not subject to the immune-dampening effects of self-tolerance.

Immune responses to neoantigens depend critically on the ability of major histocompatibility complex (MHC) molecules to effectively bind a small peptide (epitope) containing the altered amino acid sequence and present it to a T cell. Such an epitope can be generated synthetically and used in a vaccine to initiate an antigen-specific T-cell response targeting tumor cells expressing the mutated protein.

Binding of peptides to MHC can be used as a surrogate for the immunogenicity of a given peptide sequence. Advanced algorithms predicting peptide binding to MHC have been built using binding data from a large number of peptides to different MHC molecules (Lundegaard, 2011). These algorithms can be used to predict with high accuracy whether a specific peptide sequence will bind to MHC and with what affinity. Using these algorithms, protein sequences containing tumor-encoded mutations (both missense and neoORF) can be evaluated in silico for binding to a specific MHC molecule.

In some embodiments, a subject may comprise mutated epitopes comprising altered amino acid sequences, for example if the subject has cancer. In one embodiment mutated epitopes are determined by sequencing the genome and/or exome of tumor tissue and healthy tissue from a cancer patient using next generation sequencing technologies. In another embodiment genes that are selected based on their frequency of mutation and ability to act as a neoantigen are sequenced using next generation sequencing technology. Next-generation sequencing applies to genome sequencing, genome resequencing, transcriptome profiling (RNA-Seq), DNA-protein interactions (ChiP-sequencing), and epigenome characterization (de Magalhaes J P, Finch C E, Janssens G (2010). “Next-generation sequencing in aging research: emerging applications, problems, pitfalls and possible solutions”. Ageing Research Reviews 9 (3): 315-323; Hall N (May 2007). “Advanced sequencing technologies and their wider impact in microbiology”. J. Exp. Biol. 209 (Pt 9): 1518-1525; Church G M (January 2006). “Genomes for ail”. Sci. Am. 294 (1): 46-54; ten Bosch J R, Grody W W (2008). “Keeping Up with the Next Generation”. The Journal of Molecular Diagnostics 10 (6): 484-492; Tucker T, Marra M, Friedman J M (2009). “Massively Parallel Sequencing: The Next Big Thing in Genetic Medicine”. The American Journal of Human Genetics 85 (2): 142-154).

Next-generation sequencing can now rapidly reveal the presence of discrete mutations such as coding mutations in individual tumors, most commonly single amino acid changes (e.g., missense mutations) and less frequently novel stretches of amino acids generated by frame-shift insertions/deletions/gene fusions, read-through mutations in stop codons, and translation of improperly spliced introns (e.g., neoORFs). NeoORFs are particularly valuable as immunogens because the entirety of their sequence is completely novel to the immune system and so are analogous to a viral or bacterial foreign antigen. Thus, neoORFs: (1) are highly specific to the tumor (i.e. there is no expression in any normal cells); (2) can bypass central tolerance, thereby increasing the precursor frequency of neoantigen-specific CTLs. For example, the power of utilizing analogous foreign sequences in a therapeutic anti-cancer vaccine or immunogenic composition was recently demonstrated with peptides derived from human papilloma virus (HPV). −50% of the 19 patients with pre-neoplastic, viral-induced disease who received 3-4 vaccinations of a mix of HPV peptides derived from the viral oncogenes E6 and E7 maintained a complete response for >24 months (Kenter et a, Vaccination against HPV-16 Oncoproteins for Vulvar Intraepithelial Neoplasia NEJM 361: 1838 (2009)).

Sequencing technology has revealed that each tumor contains multiple, patient-specific mutations that alter the protein coding content of a gene. Such mutations create altered proteins, ranging from single amino acid changes (caused by missense mutations) to addition of long regions of novel amino acid sequence due to frame shifts, read-through of termination codons or translation of intron regions (novel open reading frame mutations; neoORFs). These mutated proteins are valuable targets for the host's immune response to the tumor as, unlike native proteins; they are not subject to the immune-dampening effects of self-tolerance. Therefore, mutated proteins are more likely to be immunogenic and are also more specific for the tumor cells compared to normal cells of the patient.

An alternative method for identifying tumor specific neoantigens is direct protein sequencing. Protein sequencing of enzymatic digests using multidimensional MS techniques (MSn) including tandem mass spectrometry (MS/MS)) can also be used to identify neoantigens of the invention. Such proteomic approaches permit rapid, highly automated analysis (see, e.g., Gevaert and J. Vandekerckhove, Electrophoresis 21: 1145-1154 (2000)). It is further contemplated within the scope of the invention that high-throughput methods for de novo sequencing of unknown proteins may be used to analyze the proteome of a patient's tumor to identify expressed neoantigens. For example, meta shotgun protein sequencing may be used to identify expressed neoantigens (see e.g., Gutilais et al. (2012) Shotgun Protein Sequencing with Meta-contig Assembly, Molecular and Cellular Proteomics 11(30): 3084-96).

Tumor specific neoantigens may also be identified using MHC multimers to identify neoantigen-specific T-cell responses. For example, high-throughput analysis of neoantigen-specific T-cell responses in patient samples may be performed using MEW tetramer-based screening techniques (see e.g., Hombrink et al. (2011) High-Throughput Identification of Potential Minor Histocompatibility Antigens by MHC Tetramer-Based Screening: Feasibility and Limitations 6(8): 1-11; Hadrup et al. (2009) Parallel detection of antigen-specific T-cell. responses by multidimensional encoding of MHC multimers, Nature Methods, 6(7):520-26; van Rooij et al. (2013) Tumor exome analysis reveals neoantigen-specific T-cell reactivity in an Ipilimumab-responsive melanoma, Journal of Clinical Oncology, 31: 1-4; and Heemskerk et al. (2013) The cancer antigenome, EMBO Journal, 32(2):194-203). Such tetramer-based screening techniques may be used for the initial identification of tumor specific neoantigens, or alternatively as a secondary screening protocol to assess what neoantigens a patient may have already been exposed to, thereby facilitating the selection of candidate neoantigens for the invention.

In one embodiment the sequencing data derived from determining the presence of mutations in a cancer patient is analyzed to predict personal mutated peptides that can bind to HLA molecules of the individual. In one embodiment the data is analyzed using a computer. In another embodiment the sequence data is analyzed for the presence of neoantigens. In one embodiment neoantigens are determined by their affinity to MHC molecules. Efficiently choosing which particular mutations to utilize as immunogen requires identification of the patient HLA type and the ability to predict which mutated peptides would efficiently bind to the patient's HLA alleles. Recently, neural network based learning approaches with validated binding and non-binding peptides have advanced the accuracy of prediction algorithms for the major HLA-A and HLA-B alleles. Utilizing the recently improved algorithms for predicting which missense mutations create strong binding peptides to the patient's cognate MHC molecules, a set of peptides representative of optimal mutated epitopes (both neoORF and missense) for each patient may be identified and prioritized (Zhang et al, Machine learning competition in immunology—Prediction of HLA class I binding peptides J Immunol Methods 374: 1 (2011); Lundegaard et al Prediction of epitopes using neural network based methods J Immunol Methods 374:26 (2011)).

Targeting as many mutated epitopes as practically possible takes advantage of the enormous capacity of the immune system, prevents the opportunity for immunological escape by down-modulation of a particular immune targeted gene product, and compensates for the known inaccuracy of epitope prediction approaches. Synthetic peptides provide a particularly useful means to prepare multiple immunogens efficiently and to rapidly translate identification of mutant epitopes to an effective vaccine or immunogenic composition. Peptides can be readily synthesized chemically and easily purified utilizing reagents free of contaminating bacteria or animal substances. The small size allows a clear focus on the mutated region of the protein and also reduces irrelevant antigenic competition from other components (unmutated protein or viral vector antigens).

In one embodiment the drug formulation is a multi-epitope vaccine or immunogenic composition of long peptides. Such “long” peptides undergo efficient internalization, processing and cross-presentation in professional antigen-presenting cells such as dendritic cells, and have been shown to induce CTLs in humans (Melief and van der Burg, Immunotherapy of established (pre) malignant disease by synthetic long peptide vaccines Nature Rev Cancer 8:351 (2008)). In one embodiment at least 1 peptide is prepared for immunization. In some embodiments, 20 or more peptides are prepared for immunization. In one embodiment the neoantigenic peptide ranges from about 5 to about 50 amino acids in length. In another embodiment peptides from about 15 to about 35 amino acids in length is synthesized. In some embodiments, the neoantigenic peptide ranges from about 20 to about 35 amino acids in length.

In some embodiments, personalized cancer vaccines consisting of up to 20 synthesized peptides approximately 14 to 35 amino acids in length that are derived from an individual patient's mutated tumor DNA (neoantigens) are provided. Because these mutations are not expressed in the patient's normal cells, they are specific targets expressed only on tumor cells.

Unlike most previously used cancer vaccines, this neoantigen peptide vaccine is based on the production of a novel and unique product for each individual patient or cancer phenotype. The extent of possible tumor mutations and the wide range of patient human leukocyte antigen (HLA) haplotypes make it highly unlikely that any 2 patients will receive the same vaccine.

Generation of neoantigens may begin with whole exome DNA and ribonucleic acid (RNA) sequencing of tumor and normal tissue samples and HLA-A, HLA-B, and HLA-C genotypes from a subject. These data may then be used to identify coding sequence mutations that have occurred in the subject's tumor. These mutations may in some cases include single-amino acid missense mutations, fusion proteins, and neoORFs which may vary in length from 1 amino acid up to hundreds of amino acids. Long peptides 14-35 residues in length may then be designed specifically from the specific mutations identified in an individual's tumor. The vaccine may then be composed of a mixture of peptides that are predicted to induce a response in CD4+ and/or CD8+ T cells. In order to predict which are most likely to induce such an immune response, a number of filters may be applied to the entire set of long peptides that cover the subject's tumor mutanome. A primary criterion is the HLA binding affinity of the mutant epitope compared to its native protein. An epitope selection algorithm may be used to identify mutation-containing epitopes that are predicted to bind to MHC class I molecules of each subject (Lundegaard, 2011). Other key criteria can include RNA expression, type of mutation (e.g., missense versus neoORF), the likelihood that the mutation is an oncogenic driver, and the physical location of the mutant residue(s) on the peptide. Up to 35 peptides may be selected and prioritized for synthesis. Thereafter, up to 20 synthesized peptides may be mixed together in up to 4 pools of up to 5 peptides each for injection. Each of the 4 pools may be injected in to the subject.

### II. Production of Tumor Specific Neoantigens

The present invention is based, at least in part, on the ability to present the immune system of the patient with a pool of tumor specific neoantigens. One of skill in the art from this disclosure a d the knowledge in the art will appreciate that there are a variety of ways in which to produce such tumor specific neoantigens. In general, such tumor specific neoantigens may be produced either in vitro or in vivo. Tumor specific neoantigens may be produced in vitro as peptides or polypeptides, which may then be formulated into a personalized neoplasia vaccine or immunogenic composition and administered to a subject. As described in further detail herein, such in vitro production may occur by a variety of methods known to one of skill in the art such as, for example, peptide synthesis or expression of a peptide/polypeptide from a DNA or RNA molecule in any of a variety of bacterial, eukaryotic, or viral recombinant expression systems, followed by purification of the expressed peptide/polypeptide. Alternatively, tumor specific neoantigens may be produced in vivo by introducing molecules (e.g., DNA, RNA, viral expression systems, and the like) that encode tumor specific neoantigens into a subject, whereupon the encoded tumor specific neoantigens are expressed. The methods of in vitro a d in vivo production of neoantigens is also further described herein as it relates to pharmaceutical compositions and methods of deliver of the combination therapy.

### A. In Vitro Peptide/Polypeptide Synthesis

Proteins or peptides may be made by any technique known to those of skill in the art, including the expression of proteins, polypeptides or peptides through standard molecular biological techniques, the isolation of proteins or peptides from natural sources, in vitro translation, or the chemical synthesis of proteins or peptides. The nucleotide and protein, polypeptide and peptide sequences corresponding to various genes have been previously disclosed, and may be found at computerized databases known to those of ordinary skill in the art. Exemplary database can be found in the National Center for Biotechnology Information, Genbank and GenPept databases located at the National Institutes of Health website. The coding regions for known genes may be amplified and/or expressed using the techniques disclosed herein or as would be known to those of ordinary skill in the art. Alternatively, various commercial preparations of proteins, polypeptides and peptides are known to those of skill in the art.

Peptides can be readily synthesized chemically utilizing reagents that are free of contaminating bacterial or animal substances (Merrifield RB: Solid phase peptide synthesis. I. The synthesis of a tetrapeptide. J. Am. Chem. Soc. 85:2149-54, 1963). In certain embodiments, neoantigenic peptides are prepared by (1) parallel solid-phase synthesis on multi-channel instruments using uniform synthesis and cleavage conditions; (2) purification over a P-HPLC column with column stripping; and re-washing, but not replacement, between peptides; followed by (3) analysis with a limited set of the most informative assays. The Good Manufacturing Practices (GMP) footprint can be defined around the set of peptides for an individual patient, thus requiring suite changeover procedures only between syntheses of peptides for different-patients.

Alternatively, a nucleic acid (e.g., a polynucleotide) encoding a neoantigenic peptide of the invention may be used to produce the neoantigenic peptide in vitro. The polynucleotide may be, e.g., DNA, cDNA, PNA, CNA, RNA, either single- and/or double-stranded, or native or stabilized forms of polynucleotides, such as e.g. polynucleotides with a phosphorothiate backbone, or combinations thereof and it may or may not contain introns so long as it codes for the peptide. In one embodiment in vitro translation is used to produce the peptide. Many exemplary systems exist that one skilled in the art could utilize (e.g., Retic Lysate IVT Kit, Life Technologies, Waltham, Mass.).

An expression vector capable of expressing a polypeptide can also be prepared. Expression vectors for different cell types are well known in the art and can be selected without undue experimentation. Generally, the DNA is inserted into an expression vector, such as a plasmid, in proper orientation and correct reading frame for expression, if necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

Expression vectors comprising the isolated polynucleotides, as well as host cells containing the expression vectors, are also contemplated. The neoantigenic peptides may be provided in the form of RNA or cDNA molecules encoding the desired neoantigenic peptides. One or more neoantigenic peptides of the invention may be encoded by a single expression vector.

The term “polynucleotide encoding a polypeptide” encompasses a polynucleotide which includes only coding sequences for the polypeptide as well as a polynucleotide which includes additional coding and/or non-coding sequences. Polynucleotides can be in the form of RNA or in the form of DNA. DNA includes cDNA, genomic DNA, and synthetic DNA; and can be double-stranded or single-stranded, and if single stranded can be the coding strand or non-coding (anti-sense) strand.

In embodiments, the polynucleotides may comprise the coding sequence for the tumor specific neoantigenic peptide fused in the same reading frame to a polynucleotide which aids, for example, in expression and/or secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide.

In embodiments, the polynucleotides can comprise the coding sequence for the tumor specific neoantigenic peptide fused in the same reading frame to a marker sequence that allows, for example, for purification of the encoded polypeptide, which may then be incorporated into the personalized neoplasia vaccine or immunogenic composition. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. Additional tags include, but are not limited to, Calmodulin tags, FLAG tags, Myc tags, S tags, SBP tags, Softag 1, Softag 3, V5 tag, Xpress tag, Isopeptag, SpyTag, Biotin Carboxyl Carrier Protein (BCCP) tags, GST tags, fluorescent protein tags (e.g., green fluorescent protein tags), maltose binding protein tags, Nus tags, Strep-tag, thioredoxin tag, TC tag, Ty tag, and the like.

In embodiments, the polynucleotides may comprise the coding sequence for one or more of the tumor specific neoantigenic peptides fused in the same reading frame to create a single concatamerized neoantigenic peptide construct capable of producing multiple neoantigenic peptides.

In certain embodiments, isolated nucleic acid molecules having a nucleotide sequence at least 60% identical, at least 65% identical, at least 70% identical, at least 75% identical, at least 80% identical, at least 85% identical, at least 90% identical, at least 95% identical, or at least 96%, 97%, 98% or 99% identical to a polynucleotide encoding a tumor specific neoantigenic peptide of the present invention, can be provided.

By a polynucleotide having a nucleotide sequence at least, for example, 95% “identical” to a reference nucleotide sequence is intended that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95%) identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the amino- or carboxy-terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 80% identical, at least 85% identical, at least 90% identical, and in some embodiments, at least 95%, 96%, 97%, 98%, or 99% identical to a reference sequence can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smit and Waterman, Advances in Applied Mathematics 2:482-489 (1981), to find the best segment of homology between two sequences. When using Bestfit or any other sequence alignment program to determine whether a particular sequence is, for instance, 95%) identical to a reference sequence according to the present invention, the parameters are set such that the percentage of identity is calculated over the full length of the reference nucleotide sequence and that gaps in homology of up to 5% of the total number of nucleotides in the reference sequence are allowed.

The isolated tumor specific neoantigenic peptides described herein can be produced in vitro (e.g., in the laboratory) by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g. Zoeller et al, Proc. Nat'l. Acad. Sci. USA 81:5662-5066 (1984) and U.S. Pat. No. 4,588,585.

In embodiments, a DNA sequence encoding a polypeptide of interest would be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest is produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (e.g., by synthesis, site-directed mutagenesis, or another method), the polynucleotide sequences encoding a particular isolated polypeptide of interest is inserted into an expression vector and optionally operatively linked to an expression control sequence appropriate for expression of the protein in a desired host. Proper assembly can be confirmed by nucleotide sequencing, restriction mapping, and expression of a biologically active polypeptide in a suitable host. As well known in the art, in order to obtain high expression levels of a transfected gene in a host, the gene can be operatively linked to transcriptional and translational expression control sequences that are functional in the chosen expression host.

Recombinant expression vectors may be used to amplify and express DNA encoding the tumor specific neoantigenic peptides. Recombinant expression vectors are replicable DNA constructs which have synthetic or cDNA-derived DNA fragments encoding a tumor specific neoantigenic peptide or a bioequivalent analog operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail herein. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Generally, operatively linked means contiguous, and in the case of secretory leaders, operatively linked means contiguous and in the reading frame. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

Useful expression vectors for eukaryotic hosts, especially mammals or humans include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR 1, pBR322, pMB9 and their derivatives, wider host range plasmids, such as Ml 3 and filamentous single-stranded DNA phages.

Suitable host cells for expression of a polypeptide include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are well known in the art (see Pouwels et al, Cloning Vectors: A Laboratory Manual, Elsevier, Y., 1985).

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include the COS-7 lines of monkey kidney cells, described by Gluzman (Cell 23: 175, 1981), and other cell lines capable of expressing an appropriate vector including, for example, L cells, C127, 3T3, Chinese hamster ovary (CHO), 293, HeLa and BHK cell lines. Mammalian expression vectors can comprise nontranscribed elements such as an origin of replication, a suitable promoter and enhancer linked to the gene to be expressed, and other 5 ‘ or 3’ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins produced by a transformed host can be purified according to any suitable method. Such standard methods include chromatography (e.g., ion exchange, affinity and sizing column chromatography, and the like), centrifugation, differential solubility, or by any other standard technique for protein purification. Affinity tags such as hexahistidine, maltose binding domain, influenza coat sequence, glutathione-S-transferase, and the like can be attached to the j 1 protein to allow easy purification by passage over an appropriate affinity column. Isolated proteins can also be physically characterized using such techniques as proteolysis, nuclear magnetic resonance and x-ray crystallography.

For example, supernatants from systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify a cancer stem cell protein-Fc composition. Some or all of the foregoing purification steps, in various combinations, can also be employed to provide a homogeneous recombinant protein.

Recombinant protein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

### B. In Vivo Peptide/Polypeptide Synthesis

The present invention also contemplates the use of nucleic acid molecules as vehicles for delivering neoantigenic peptides/polypeptides to the subject in need thereof, in vivo, in the form of, e.g., DNA/RNA vaccines (see, e.g., WO2012/159643, and WO2012/159754, hereby incorporated by reference in their entirety).

In one embodiment neoantigens may be administered to a patient in need thereof by use of a plasmid. These are plasmids which usually consist of a strong viral promoter to drive the in vivo transcription and translation of the gene (or complementary DNA) of interest (Mor, et al., (1995). The Journal of Immunology 155 (4): 2039-2046). Intron A may sometimes be included to improve mRNA stability and hence increase protein expression (Leitner et al, (1997). The Journal of Immunology 159 (12): 6112-6119). Plasmids also include a strong polyadenylation/transcriptional termination signal, such as bovine growth hormone or rabbit beta-globulin polyadenylation sequences (Alarcon et al, (1999). Adv. Parasitol. Advances in Parasitology 42: 343-410; Robinson et al., (2000). Adv. Virus Res. Advances in Virus Research 55: 1-74; Bohm et al, (1996). Journal of Immunological Methods 193 (i): 29-40.). Multicistronic vectors are sometimes constructed to express more than one immunogen, or to express an immunogen and an immunostimulatory protein (Lewis et al., (1999). Advances in Virus Research (Academic Press) 54: 129-88).

Because the plasmid is the “vehicle” from which the immunogen is expressed, optimizing vector design for maximal protein expression is essential (Lewis et al., (1999), Advances in Vims Research (Academic Press) 54: 129-88). One way of enhancing protein expression is by optimizing the codon usage of pathogenic mRNAs for eukaryotic cells. Another consideration is the choice of promoter. Such promoters may be the SV40 promoter or Rous Sarcoma Vims (RSV),

Plasmids may be introduced into animal tissues by a number of different methods. The two most popular approaches are injection of DNA in saline, using a standard hypodermic needle, and gene gun delivery, A schematic outline of the construction of a DNA vaccine plasmid and its subsequent delivery by these two methods into a host is illustrated at Scientific American (Weiner et al., (1999) Scientific American 281 (I): 34-41). Injection in saline is normally conducted intramuscularly (IM) in skeletal muscle, or intradermally (ID), with DNA being delivered to the extracellular spaces. This can be assisted by electroporation by temporarily damaging muscle fibers with myotoxins such as bupivacaine; or by using hypertonic solutions of saline or sucrose (Alarcon et al., (1999). Adv. Parasitol. Advances in Parasitology 42: 343-410). Immune responses to this method of delivery can be affected by many factors, including needle type, needle alignment, speed of injection, volume of injection, muscle type, and age, sex and physiological condition of the animal being injected (Alarcon et al., (1999). Adv. Parasitol. Advances in Parasitology 42: 343-410).

Gene gun delivery, the other commonly used method of delivery, ballistically accelerates plasmid DNA (pDNA) that has been adsorbed onto gold or tungsten microparticles into the target cells, using compressed helium as an accelerant (Alarcon et al., (1999), Adv, Parasitol. Advances in Parasitology 42: 343-410; Lewis et al., (1999), Advances in Virus Research (Academic Press) 54: 12.9-88),

Alternative delivery methods may include aerosol instillation of naked DNA on mucosal surfaces, such as the nasal and lung mucosa, (Lewis et al., (1999). Advances in Virus Research (Academic Press) 54: 129-88) and topical administration of pDNA to the eye and vaginal mucosa (Lewis et al., (1999) Advances in Vims Research (Academic Press) 54: 129-88). Mucosal surface delivery has also been achieved using cationic liposome-DNA preparations, biodegradable microspheres, attenuated Shigella or Listeria vectors for oral administration to the intestinal mucosa, and recombinant adenovirus vectors,

The method of delivery determines the dose of DNA required to raise an effective immune response. Saline injections require variable amounts of DNA, from 10 μg-1 mg, whereas gene gun deliveries require 100 to 1000 times less DNA than intramuscular saline injection to raise an effective immune response. Generally, 0.2 μg-20 μg are required, although quantities as low as 16 ng have been reported. These quantities vary from species to species, with mice, for example, requiring approximately 10 times less DNA than primates. Saline injections require more DNA because the DNA is delivered to the extracellular spaces of the target tissue (normally muscle), where it has to overcome physical barriers (such as the basal lamina and large amounts of connective tissue, to mention a few) before it is taken up by the cells, while gene gun deliveries bombard DNA directly into the cells, resulting in less “wastage” (See e.g., Sedegah et al., (1994). Proceedings of the National Academy of Sciences of the United States of America 91 (21): 9866-9870; Daheshia et al., (1997). The Journal of Immunology 159 (4): 1945-1952; Chen et al., (1998). The Journal of Immunology 160 (5): 2425-2432; Sizemore (1995) Science 270 (5234): 299-302; Fynan et al., (1993) Proc. Natl. Acad. Sci. U.S.A. 90 (24): 11478-82).

In one embodiment, a neoplasia vaccine or immunogenic composition may include separate DNA plasmids encoding, for example, one or more neoantigenic peptides/polypeptides as identified in according to the invention. As discussed herein, the exact choice of expression vectors can depend upon the peptide/polypeptides to be expressed, and is well within the skill of the ordinary artisan. The expected persistence of the DNA constructs (e.g., in an episomal, non-replicating, non-integrated form in the muscle cells) is expected to provide an increased duration of protection.

One or more neoantigenic peptides of the invention may be encoded and expressed in vivo using a viral based system (e.g., an adenovirus system, an adeno associated vims (AAV) vector, a poxvirus, or a lentivirus). In one embodiment, the neoplasia vaccine or immunogenic composition may include a viral based vector for use in a human patient in need thereof, such as, for example, an adenovirus (see, e.g., Baden et al. First-in-human evaluation of the safety and immunogenicity of a recombinant adenovirus serotype 26 HIV-1 Env vaccine (1PCAVD 001). J Infect Dis. 2013 Jan. 15; 207(2):240-7, hereby incorporated by reference in its entirety). Plasmids that can be used for adeno associated virus, adenovirus, and lentivirus delivery have been described previously (see e.g., U.S. Pat. Nos. 6,955,808 and 6,943,019, and U.S. Patent application No. 20080254008, hereby incorporated by reference).

Among vectors that may be used in the practice of the invention, integration in the host genome of a cell is possible with retrovirus gene transfer methods, often resulting in long term expression of the inserted transgene. In some embodiments, the retrovirus is a lentivirus. Additionally, high transduction efficiencies have been observed in many different cell types and target tissues. The tropism of a retrovirus can be altered by incorporating foreign envelope proteins, expanding the potential target population of target cells. A retrovirus can also be engineered to allow for conditional expression of the inserted transgene, such that only certain cell types are infected by the lentivirus. Cell type specific promoters can be used to target expression in specific cell types. Lentiviral vectors are retroviral vectors (and hence both lentiviral and retroviral vectors may be used in the practice of the invention). Moreover, lentiviral vectors are able to transduce or infect non-dividing cells and typically produce high viral titers. Selection of a retroviral gene transfer system may therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the desired nucleic acid into the target cell to provide permanent expression. Widely used retroviral vectors that may be used in the practice of the invention include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., (1992) J. Virol. 66:2731-2739; Johann et al., (1992) J. Virol. 66: 1635-1640; Sommnerfelt et al., (1990) Virol. 176:58-59; Wilson et al, (1998) J. Virol. 63:2374-2378; Miller et al., (1991) J. Virol. 65:2220-2224; PCT/US94/05700). Zo et al. administered about 10 μl of a recombinant lentivirus having a titer of 1×10⁹ transducing units (TU)/ml by an intrathecal catheter. These sort of dosages can be adapted or extrapolated to use of a retroviral or lentiviral vector in the present invention.

Also useful in the practice of the invention is a minimal non-primate lentiviral vector, such as a lentiviral vector based on the equine infectious anemia vims (EIAV) (see, e.g., Balagaan, (2006) J Gene Med; 8: 275-285, Published online 21 Nov. 2005 in Wiley InterScience (interscience.wiley.com). DOI: 1002/jgm.845). The vectors may have cytomegalovirus (CMV) promoter driving expression of the target gene. Accordingly, the invention contemplates amongst vector(s) useful in the practice of the invention: viral vectors, including retroviral vectors and lentiviral vectors.

Also useful in the practice of the invention is an adenovirus vector. One advantage is the ability of recombinant adenoviruses to efficiently transfer and express recombinant genes in a variety of mammalian cells and tissues in vitro and in vivo, resulting in the high expression of the transferred nucleic acids. Further, the ability to productively infect quiescent cells expands the utility of recombinant adenoviral vectors. In addition, high expression levels ensure that the products of the nucleic acids will be expressed to sufficient levels to generate an immune response (see e.g., U.S. Pat. No. 7,029,848, hereby incorporated by reference).

In an embodiment herein the delivery is via an adenovirus, which may be at a single booster dose containing at least 1×10⁵ particles (also referred to as particle units, pu) of adenoviral vector. In an embodiment herein, the dose can be at least about 1×10⁶ particles (for example, about 1×10⁶-1×10² particles), at least about 1×10⁷ particles, at least about 1×10⁸ particles (e.g., about 1×10⁸-1×10¹¹ particles or about 1×10⁸-1×10¹² particles), or at least about 1×10⁹ particles (e.g., about 1×10⁹-1×10¹⁰ particles or about 1×10⁹-1×10¹² particles), or even at least about 1×10¹⁰ particles (e.g., about 1×10¹⁰-1×10¹² particles) of the adenoviral vector. Alternatively, the dose comprises no more than about 1×10¹⁴ particles, no more than about 1×10¹³ particles, no more than about 1×10¹² particles, no more than about 1×10¹¹ particles, or no more than about 1×10¹⁰ particles (e.g., no more than about 1×10⁹ articles). Thus, the dose may contain a single dose of adenoviral vector with, for example, about 1×10⁶ particle units (pu), about 2×10⁶ pu, about 4×10⁶ pu, about 1×10⁷ pu, about 2×10⁷ pu, about 4×10⁷ pu, about 1×10⁸ pu, about 2×10⁸ pu, about 4×10⁸ pu, about 1×10⁹ pu, about 2×10⁹ pu, about 4×10⁹ pu, about 1×10¹⁰ pu, about 2×10¹⁰ pu, about 4×10¹⁰ pu, about 1×10¹¹ pu, about 2×10¹¹ pu, about 4×10¹¹ pu, about 1×10¹² pu, about 2×10¹² pu, or about 4×10¹² pu of adenoviral vector. See, for example, the adenoviral vectors in U.S. Pat. No. 8,454,972 B2 to Nabel, et. al, granted on Jun. 4, 2013; incorporated by reference herein, and the dosages at col 29, lines 36-58 thereof In an embodiment herein, the adenovirus is delivered via multiple doses.

In terms of in vivo delivery, AAV is advantageous over other viral vectors due to low toxicity and low probability of causing insertional mutagenesis because it doesn't integrate into the host genome. AAV has a packaging limit of 4.5 or 4.75 Kb. Constructs larger than 4.5 or 4.75 Kb result in significantly reduced virus production. There are many promoters that can be used to drive nucleic acid molecule expression. AAV ITR can serve as a promoter and is advantageous for eliminating the need for an additional promoter element. For ubiquitous expression, the following promoters can be used: CMV, CAG, CBh, PGK, SV40, Ferritin heavy or light chains, etc. For brain expression, the following promoters can be used: SynapsinI for all neurons, CaMKIIalpha for excitatory neurons, GAD67 or GAD65 or VGAT for GABAergic neurons, etc. Promoters used to drive RNA synthesis can include: Pol II I promoters such as U6 or HI, The use of a Pol II promoter and intronic cassettes can be used to express guide RNA (gRNA).

As to AAV, the AAV can be AAV1, AAV2, AAV5 or any combination thereof. One can select the AAV with regard to the cells to be targeted; e.g., one can select AAV serotypes 1, 2, 5 or a hybrid capsid AAV 1, AAV2, AAV5 or any combination thereof for targeting brain or neuronal cells; and one can select AAV4 for targeting cardiac tissue. AAV8 is useful for delivery to the liver. The above promoters and vectors can be used individually.

In an embodiment herein, the delivery is via an AAV. A therapeutically effective dosage for in vivo delivery of the AAV to a human is believed to be in the range of from about 20 to about 50 ml of saline solution containing from about 1×10¹⁰ to about 1×10⁵⁰ functional AAV/ml solution. The dosage may be adjusted to balance the therapeutic benefit against any side effects. In an embodiment herein, the AAV dose is generally in the range of concentrations of from about 1×10⁵ to 1×10⁵⁰ genomes AAV, from about 1×10⁸ to 1×10²⁰ genomes AAV, from about 1×10¹⁰ to about 1×10¹⁶ genomes, or about 1×10¹¹ to about 1×10¹⁶ genomes AAV. A human dosage may be about 1×10¹³ genomes AAV. Such concentrations may be delivered in from about 0.001 ml to about 100 ml, about 0.05 to about 50 ml, or about 10 to about 25 ml of a carrier solution. In some embodiments, AAV is used with a titer of about 2×10¹³ viral genomes/milliliter, and each of the striatal hemispheres of a mouse receives one 500 nanoliter injection. Other effective dosages can be readily established by one of ordinary skill in the art through routine trials establishing dose response curves. (See, e.g., U.S. Pat. No. 8,404,658 B2, hereby incorporated by reference in its entirety).

In another embodiment effectively activating a cellular immune response for a neoplasia vaccine or immunogenic composition can be achieved by expressing the relevant neoantigens in a vaccine or immunogenic composition in a non-pathogenic microorganism. Well-known examples of such microorganisms are Mycobacterium bovis BCG, Salmonella and Pseudomona (See, U.S. Pat. No. 6,991,797, hereby incorporated by reference in its entirety).

In another embodiment a Poxvirus is used in the neoplasia vaccine or immunogenic composition. These include orthopoxvirus, avipox, vaccinia, MVA, NYVAC, canarypox, ALVAC, fowlpox, TROVAC, etc. (See e.g., Verardiet al., Hum Vaccin Immunother. 2012 Jul.; 8 (7):961-70; and Moss, Vaccine. 2013: 31(39): 4220-4222). Poxvirus expression vectors were described in 1982 and quickly became widely used for vaccine development as well as research in numerous fields. Advantages of the vectors include simple construction, ability to accommodate large amounts of foreign DNA and high expression levels.

In another embodiment the vaccinia virus is used in the neoplasia vaccine or immunogenic composition to express a neoantigen. (See e.g., Rolph et al., Recombinant viruses as vaccines and immunological tools. Curr Opin Immunol 9:517-524, 1997). The recombinant vaccinia virus is able to replicate within the cytoplasm of the infected host cell and the polypeptide of interest can therefore induce an immune response. Moreover, Poxviruses have been widely used as vaccine or immunogenic composition vectors because of their ability to target encoded antigens for processing by the major histocompatibility complex class I pathway by directly infecting immune cells, in particular antigen-presenting cells, but also due to their ability to self-adjuvant.

In another embodiment ALVAC is used as a vector in a neoplasia vaccine or immunogenic composition. ALVAC is a canarypox vims that can be modified to express foreign transgenes and has been used as a method for vaccination against both prokaryotic and eukaryotic antigens (Honig H, Lee D S, Conkright W, et al. Phase I clinical trial, of a recombinant canarypoxvirus (ALVAC) vaccine expressing human carcinoembryonic antigen and the B7.1 co-stimulatory molecule. Cancer Immunol Immunother 2000; 49:504-14; von Mehren M, Arlen P, Tsang K Y, et al. Pilot study of a dual gene recombinant avipox vaccine containing both carcinoembryonic antigen (CEA) and B7.1 transgenes in patients with recurrent CEA-expressing adenocarcinomas. Clin Cancer Res 2000; 6:2219-28; Musey L, Ding Y, Elizaga M, et al. HIV-1 vaccination administered intramuscularly can induce both, systemic and mucosal T cell immunity in HIV-1-uninfected individuals. J Immunol 2003; 171:1094-101; Paoletti E. Applications of pox virus vectors to vaccination: an update. Proc Natl Acad Sci USA 1996; 93:11349-53; U.S. Pat. No. 7,255,862). In a phase I clinical trial, an ALVAC virus expressing the tumor antigen CEA showed an excellent safety profile and resulted in increased CEA-specific T-cell responses in selected patients; objective clinical responses, however, were not observed (Marshall J L, Hawkins M J, Tsang K Y, et al. Phase I study in cancer patients of a replication-defective avipox recombinant, vaccine that expresses human carcinoembryonic antigen. J Clin Oncol 1999; 17:332-7).

In another embodiment a Modified Vaccinia Ankara (MVA) virus may be used as a viral vector for a neoantigen vaccine or immunogenic composition. MVA is a member of the Orthopoxvirus family and has been generated by about 570 serial passages on chicken embryo fibroblasts of the Ankara strain of Vaccinia virus (CVA) (for review see Mayr, A., et al., Infection 3, 6-14, 1975). As a consequence of these passages, the resulting MVA vims contains 3.1 kilobases less genomic information compared to CVA, and is highly host-cell restricted (Meyer, H. et al., J. Gen. Virol. 72, 1031-1038, 1991). MVA is characterized by its extreme attenuation, namely, by a diminished virulence or infectious ability, but still holds an excellent immunogenicity. When tested in a variety of animal models, MVA was proven to be avirulent, even in immuno-suppressed individuals. Moreover, MVA-BN®-HER2 is a candidate immunotherapy designed for the treatment of HER-2-positive breast, cancer and is currently in clinical trials. (Mandl et al. Cancer Immunol Immunother. January 2012; 61(1): 19-29). Methods to make and use recombinant MVA have been described (e.g., see U.S. Pat. Nos. 8,309,098 and 5,185,146 hereby incorporated in its entirety).

In another embodiment the modified Copenhagen strain of vaccinia virus, NYVAC and NYVAC variations are used as a vector (see U.S. Pat. No. 7,255,862; PCT WO 95/30018; U.S. Pat. Nos. 5,364,773 and 5,494,807, hereby incorporated by reference in its entirety),

In one embodiment recombinant viral particles of the vaccine or immunogenic composition are administered to patients in need thereof. Dosages of expressed neoantigen can range from a few to a few hundred micrograms, e.g., 5 to 500 .mu.g. The vaccine or immunogenic composition can be administered in any suitable amount to achieve expression at these dosage levels. The viral particles can be administered to a patient in need thereof or transfected into cells in an amount of about at least 10^{3.5} pfu; thus, the viral particles can be administered to a patient in need thereof or infected or transfected into cells in at least about 10⁴ pfu to about 10⁶ pfu; however, a patient in need thereof can be administered at least about 10⁸ pfu such that an amount for administration can be at least about 10⁷ pfu to about 10⁹ pfu. Doses as to NYVAC are applicable as to ALVAC, MVA, MVA-BN, and avipoxes, such as canarypox and fowlpox.

### III. Vaccine or Immunogenic Composition Adjuvant

Toll-like receptors (TLRs) are important members of the family of pattern recognition receptors (PRRs) expressed by cells of the innate and adaptive immune systems. The TLRs recognize conserved motifs shared by many microorganisms, termed pathogen-associated molecular patterns (PAMPS). Different TLRs recognize distinct PAMPs, and TLR ligand binding leads to activation of inflammatory signaling cascades including the nuclear factor kappa light-chain of activated B cells (NF-κB) transcription factor and the type I interferons (IFNs). Toll-like receptor-mediated activation of APCs such as dendritic cells (DCs) results in increased expression of MHC and T-cell co-stimulatory molecules and can help facilitate initiation of a peptide-specific T-cell response.

Non-limiting examples of cancer vaccine adjuvants include TLR9 agonist 5′-C-phoshate-G-3′ (CpG) and the synthetic double-stranded ribonucleic acid (dsRNA) TLR3 ligand polyinosinic-polycytidylic acid-polylysine carboxymethylcellulose (adjuvant) (poly-ICLC) [Hiltonol®] (poly-inosinic acid: poly-cytidilic acid). The CpG is a synthetic dinucleotide, and pICLC is a synthetic, dsRNA stabilized with poly-lysine and carboxymethylcellulose.

Poly-ICLC is a synthetic, dsRNA “host-targeted” therapeutic viral-mimic and PAMP with broad innate and adaptive immune adjuvant function. Poly-ICLC exerts its function through TLR3, melanoma differentiation-associate protein 5 (MDA5), and several nuclear and cytoplasmic enzyme systems (oligoadenylate synthetase, the dsRNA-dependent protein kinase R [PKR], retinoic acid-inducible gene 1 [RIG-1] helicase, and MDA5) that are involved in antiviral and anti-tumor host defenses.

Stimulation with poly-ICLC leads to DC and natural killer (NK) cell activation and production of a natural mix of type I IFNS, cytokines, and chemokines (Meylan, 2006). This adjuvant has been shown to induce local and systemic activation of immune cells in vivo, produce stimulatory chemokines and cytokines, and stimulate antigen presentation by DCs. In preclinical studies, poly-ICLC appears to be a potent TLR adjuvant due to its induction of pro-inflammatory cytokines, lack of stimulation of Interleukin-10 (IL-10), and maintenance of high levels of co-stimulatory molecules in DCs (Bogunovic, 2011). Furthermore, poly-ICLC was directly compared to CpG in non-human primates as an adjuvant for a protein vaccine consisting of human papillomavirus (HPV) 16 capsomers and was found to be much more effective in inducing HPV-specific T_{H}1 (T helper cell 1) immune responses (Stahl-Hennig, 2009).

Poly-ICLC can induce durable CD4+ and CD8+ responses in humans. Striking similarities were seen in the up-regulation of transcriptional and signal transduction pathways between patients vaccinated with poly-ICLC and in volunteers who had received the highly effective, replication-competent yellow fever vaccine (Okada, 2011). In a recent Phase 1 study, >90% of ovarian carcinoma patients immunized with poly-ICLC in combination with a NY-ESO-1 peptide vaccine showed induction of CD4+ and CD8+ T cells, as well as antibody responses to the peptide (Sabbatini, 2012). Without being bound by theory, these neoantigens are expected to bypass central thymic tolerance (thus allowing stronger anti-tumor T cell response), while reducing the potential for autoimmunity (e.g., by avoiding targeting of normal self-antigens). An effective immune response advantageously includes a strong adjuvant to activate the immune system (Speiser and Romero, Molecularly defined vaccines for cancer immunotherapy, and protective T cell immunity Seminars in Immunol 22: 144 (2010)). For example, Toll-like receptors (TLRs) have emerged as powerful sensors of microbial and viral pathogen “danger signals”, effectively inducing the innate immune system, and in turn, the adaptive immune system (Bhardwaj and Gnjatic, TLR AGONISTS: Are They Good Adjuvants? Cancer J. 16:382-391 (2010)). Among the TLR agonists, poly-ICLC (a synthetic double-stranded RNA mimic) is one of the most potent activators of myeloid-derived dendritic cells. In a human volunteer study, poly-ICLC has been shown to be safe and to induce a gene expression profile in peripheral blood cells comparable to that induced by one of the most potent live attenuated viral vaccines, the yellow fever vaccine YF-17D (Caskey et al, Synthetic double-stranded RNA induces innate immune responses similar to a live viral vaccine in humans J Exp Med 208:2357 (2011)). In some embodiments, Hiitonol®, a GMP preparation of poly-ICLC prepared by Oncovir, Inc. is utilized as the adjuvant. In other embodiments, other adjuvants described herein are envisioned. For instance oil-in-water, water-in-oil or multiphasic W/O/W; see, e.g., U.S. Pat. No. 7,608,279 and Aucouturier et al, Vaccine 19 (2001), 2666-2672, and documents cited therein.

### IV. Immune Checkpoint Modulators

Immune checkpoints are crucial signaling pathways in the immune system that maintain self-tolerance and modulate the duration and amplitude of physiological immune responses. Under normal conditions, these pathways prevent excessive effector activity by T cells. Two important examples of this pathway are the cell surface receptors CTLA-4 and PD-1 (Teft, 2006; Keir, 2008). In some cases, tumors express or over-express inhibitory immune checkpoint pathways as a major mechanism of immune evasion. Because many of the immune checkpoints are initiated by ligand-receptor interactions, these signals can be readily blocked by antibodies or modulated by recombinant forms of ligands or receptors (Pardoll, 2012).

Immune checkpoint proteins are important targets for pharmacologic blockade (Teft, 2006; Keir, 2008), and dramatic clinical responses have been observed after treatment with antibodies blocking PD-1 and CTLA-4 (See, e.g., Brahmer, 2010; Robert, 2011; Topalian, 2012; Powles, 2014; Topalian, 2014; Brahmer, 2015; Le, 2015; Robert, 2015; Reck, 2016; Langer, 2017). Accordingly, the present invention features in exemplary embodiments, novel combinations of a neoplasia vaccine or immunogenic composition and one or more anti-CTLA4 or anti-PD-1 antibodies.

In some embodiments, the anti-CTLA4 antibody is Ipilimumab.Anti-CTLA-4 antibody, or ipilimumab, can be a recombinant, human monoclonal antibody that binds to the CTLA-4 and blocks the interaction of CTLA-4 with its ligands, CD80/CD86. Blockade of CTLA-4 has been shown to augment T-cell activation and proliferation, including the activation and proliferation of tumor-infiltrating T effector cells. Inhibition of CTLA-4 signaling can also reduce T regulatory cell function, which may contribute to a general increase in T-cell responsiveness, including the anti-tumor immune response. Ipilimumab is an IgG1 kappa immunoglobulin with an approximate molecular weight of 148 kDa. Ipilimumab (Yervoy®′ Bristol-Meyers Squibb, New York, N.Y.) is a recombinant, human mAb produced in mammalian (Chinese hamster ovaries) cell culture.

CTLA-4 serves to regulate early T cell activation and Programmed Death-1 (PD-1) signaling functions in part to regulate T cell activation in peripheral tissues. The PD-1 receptor refers to an immunoinhibitory receptor belonging to the CD28 family. PD-1 is expressed on a number of cell types including Tregs, activated B cells, and natural killer (NK) cells, and is expressed predominantly on previously activated T cells in vivo, and binds to two ligands, PD-L1 and PD-L2. PD1 's endogenous ligands, PD-L1 and PD-L2, are expressed in activated immune cells as well as nonhematopoietic cells, including tumor cells. PD-1 as used herein is meant to include human PD-1 (hPD-1), variants, isoforms, and species homologs of hPD-1, and analogs having at least one common epitope with hPD-1. The complete hPD-1 sequence can be found under GENBANK Accession No. U64863. Programmed Death Ligand-1 (PD-L1 “is one of two cell surface glycoprotein ligands for PD-1 (the other being PD-L2) that downregulate T cell activation and cytokine secretion upon binding to PD-1. PD-L3 as used herein includes human PD-L1 (hPD-L1), variants, isoforms, and species homologs of hPD-L1, and analogs having at least one common epitope with hPD-L1. The complete hPD-L1 sequence can be found under GENBAN Accession No. Q9NZQ7. Tumors have been demonstrated to escape immune surveillance by expressing PD-L1/L2, thereby suppressing tumor-infiltrating lymphocytes via PD-1/PD˜L1,2 interactions (Dong et al. Nat. Med. 8:793-800. 2002).

In some embodiments, the anti-PD-1 antibody is nivolumab. Nivolumab (Opdivo®, Bristol-Myers Squibb Company, NY) is a human immunoglobulin G4 (IgG4) mAb that binds to the programmed death 1 (PD-1) receptor and blocks its interaction with PD-L1 and programmed death ligand 2 (PD-L2), reversing PD-1 pathway-mediated inhibition of the immune response, including the anti-tumor immune response. Binding of PD-L1 and PD-L2 to the PD-1 receptor expressed on T cells inhibits T-cell proliferation and cytokine production. Up-regulation of PD-1 ligands occurs in some tumors, and signaling through this pathway can contribute to inhibition of active T-cell immune surveillance of tumors. The antibodies of the invention includes, but are not limited to, all of the anti-PD-1 and anti-PD-L1 Abs disclosed in U.S. Pat. Nos. 8,008,449 and 7,943,743, respectively. Other anti-PD-1 mAbs have been described in, for example, U.S. Pat. Nos. 7,488,802 and 8,168,757, and anti-PD-L1 mAbs have been described in, for example, U.S. Pat. Nos. 7,635,757 and 8,217,149, and U.S. Publication No. 2009/0317368. U.S. Pat. No. 8,008,449 exemplifies seven anti-PD-1 HuMAbs: 1 708. 2D3, 4M, 5C4 (also referred to herein as nivolumab or BMS-936558), 4A11, 7D3 and 5F4.

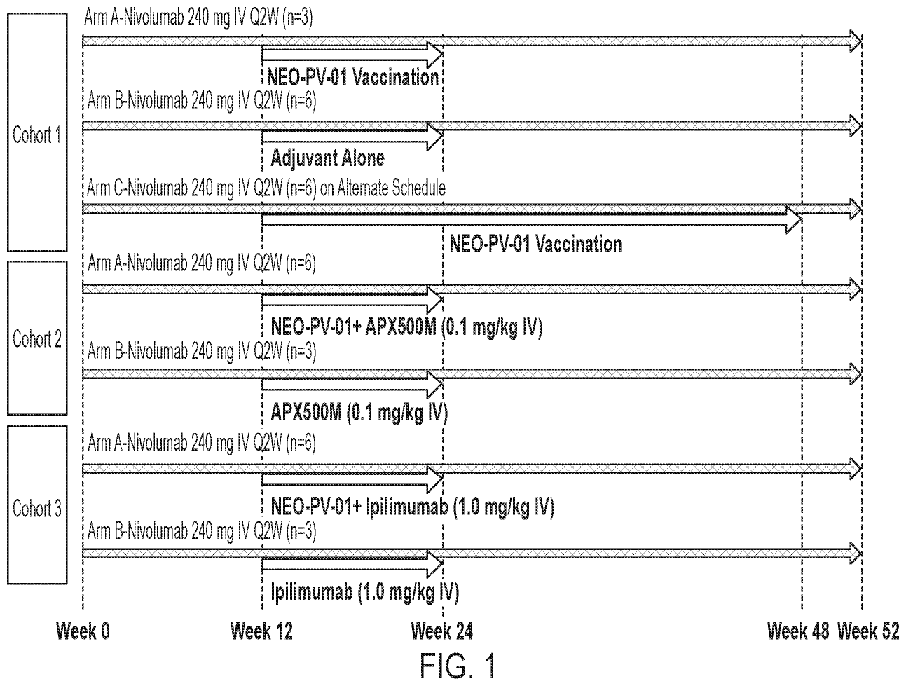

In addition to CTLA-4 and PD-1/PD-L1, numerous other immunomodulatory targets have been identified preliminarily, many with corresponding therapeutic antibodies that are being investigated in clinical trials. Page et al. (Annu. Rev. Med. 2014.65) details targets of antibody immune modulators in FIG. 1, incorporated by reference herein,

The present invention features in exemplary aspects, novel combinations of a neoplasia vaccine or immunogenic composition and one or more inhibitors of the PD-1 pathway. In some embodiments, the inhibitor of the PD-1 pathway is an anti-PD1 antibody, for example Nivolumab.

The present invention also features in other exemplary aspects, novel combinations of a neoplasia vaccine or immunogenic composition and Nivolumab and/or one or more anti-CTLA4 antibodies.

In further additional embodiments, the inhibitor is targeted at a member of the TNFR superfamily such as CD40, OX40, CD137, GITR, CD27 or TIM-3. In some cases the inhibitor is an antibody or similar molecule. In other cases, the inhibitor is an agonist for the target; examples of this class include the stimulatory targets OX40 and GITR. CD40 is a member of the tumor necrosis factor receptor (TNFR) superfamily and plays a role in induction of tumor apoptosis and regulation of immune activation, especially in crosstalk between T cells and antigen-presenting cells (APCs) (Aggarwal, 2003). CD40 ligand (CD40L), also known as CD154, is the chief ligand described for CD40 and is expressed primarily by activated T lymphocytes and platelets (Grewal, 1998). CD40 is expressed on APCs such as DCs, B cells, monocytes (FIG. 1), and other nonlymphoid cells (Banchereau, 1994). CD40-agonistic antibodies can substitute for CD40L/CD154 on activated T cells to boost immunity.

Signaling through CD40 on APCs, including DCs, B cells, and monocytes, results in improved antigen processing and presentation, and cytokine release from activated APCs, which in turn enhance the T-cell response (Clark, 1994; Grewal, 1998). Because of its action on both immune and tumor cells, CD40 has been studied as a target for novel cancer immunotherapy; agonistic anti-CD40 antibodies have been demonstrated to be potent stimulators of tumor immune responses in both animal models and cancer patients (Khong, 2012; Law, 2009; Rakhmilevich, 2012; Tong, 2003).

In some embodiments, an anti-CD40 antibody may be used. APX005M is an IgG1 humanized mAb with the S267E mutation at the Fc region. APX005M binds with high affinity to human CD40 (Kd=1.2×10⁻¹° M) and monkey CD40 (Kd=3.5×10⁻¹⁰ M), but does not cross-react with mouse or rat CD40. APX005M blocks the binding of CD40 to CD40L.

Preclinical experiments with APX005M showed that it activates the CD40 signaling pathway, leading to APC activation, as demonstrated by an increased expression of CD80, CD83, and CD86 and by expression and release of cytokines from human DCs and lymphocytes. As a result of APC activation, APX005M enhances T-cell proliferation to alloantigen, triggers production of IFN-γ in response to viral antigens, and enhances T-cell response to tumor antigens. APX005M did not appear to have a substantive effect on normal human DC and T-cell counts, but could partially reduce B-cell counts in vitro. The potential for APX005M to induce expression of cytokines was evaluated with peripheral blood mononuclear cells (PBMCs) obtained from normal humans and treatment-naïve cynomolgus monkeys, including anti-CD3 antibody as a positive control. Cytokine secretion differed significantly between species with much less secretion from monkey PBMCs compared with human PBMCs. These data suggest that APX005M is a strong CD40 agonistic antibody that can activate APCs (DCs, B cells, and monocytes) and in turn stimulate T-cell response. APX005M demonstrated a dose-dependent activation of APCs (as demonstrated by increases in expression of activation markers such as CD54, CD70, CD80, CD86, and HLA-DR), T-cell activation and increases in circulating levels of IL-12, INF-γ, TNF-α, and IL-6.

Administration of the vaccine or immunogenic composition may be combined with administration of one or more inhibitors, such as one or more checkpoint inhibitors or CD40 agonists. In some embodiments, administration of the neoantigenic vaccine composition is combined with the administration of two inhibitors, such as two checkpoint inhibitor or two CD40 agonists. For instance, a regimen of neoantigen administration may be combined with the administration of a CTLA4 inhibitor such as ipilimumab and a PD-1 inhibitor such as nivolumab. In some cases, a regimen of neoantigen administration may be combined with the administration of nivolumab and a CD40 inhibitor such as APX005M. In some cases, a regimen of neoantigen administration may be combined with the administration of ipilimumab and APX005M.

In some embodiments, a CD40 agonist antibody such as APX005M may be administered once or more than once along with neoantigen administration. In some dosage regimens, a CD40 agonist antibody such as APX005M may be administered once as a priming dose at the beginning of the vaccination period, followed by one, two, three, four, five or more boost doses during and/or after the neoantigen vaccine doses. In some embodiments, a CD40 agonist antibody dose is administered once before administering the neoantigen vaccine. In some embodiments, a CD40 agonist antibody dose is administered more than once before administering the neoantigen vaccine. In some embodiments, a CD40 agonist antibody dose is administered once after administering the neoantigen vaccine. In some embodiments, a CD40 agonist antibody dose is administered more than once after administering the neoantigen vaccine. In some embodiments, a CD40 agonist antibody dose is administered twice, three times, four times, five times or more after administering the neoantigen vaccine. In some embodiments, a CD40 agonist antibody dose is administered both before and after administering the neoantigen vaccine.

In some embodiments, a CTLA4 inhibitor such as ipilimumab may be administered once or more than once along with neoantigen administration. In some dosage regimens, a CTLA4 inhibitor such as ipilimumab may be administered once as a priming dose at the beginning of the vaccination period, followed by one, two, three, four, five or more boost doses during and/or after the neoantigen vaccine doses. In some embodiments, an ipilimumab dose is administered once before administering the neoantigen vaccine. In some embodiments, an ipilimumab dose is administered more than once before administering the neoantigen vaccine. In some embodiments, an ipilimumab dose is administered once after administering the neoantigen vaccine. In some embodiments, an ipilimumab dose is administered more than once after administering the neoantigen vaccine. In some embodiments, an ipilimumab dose is administered twice, three times, four times, five times or more after administering the neoantigen vaccine. In some embodiments, an ipilimumab dose is administered both before and after administering the neoantigen vaccine.

In some embodiments, a PD-1 inhibitor such as nivolumab may be administered once or more than once along with neoantigen administration. In some dosage regimens, a PD-1 inhibitor such as nivolumab may be administered once as a priming dose at the beginning of the vaccination period, followed by one, two, three, four, five or more boost doses during and/or after the neoantigen vaccine doses. In some embodiments, a nivolumab dose is administered once before administering the neoantigen vaccine. In some embodiments, a nivolumab dose is administered more than once before administering the neoantigen vaccine. In some embodiments, a nivolumab dose is administered once after administering the neoantigen vaccine. In some embodiments, a nivolumab dose is administered more than once after administering the neoantigen vaccine. In some embodiments, a nivolumab dose is administered twice, three times, four times, five times or more after administering the neoantigen vaccine. In some embodiments, a nivolumab dose is administered both before and after administering the neoantigen vaccine.

In some embodiments, the subject is suffering from a neoplasia selected from the group consisting of Non-Hodgkin's Lymphoma (NHL), clear cell Renal Cell Carcinoma (ccRCC), melanoma, sarcoma, leukemia or a cancer of the bladder, colon, brain, breast, head and neck, endometrium, lung, ovary, pancreas or prostate. In some embodiments, the neoplasia is metastatic melanoma. In some embodiments, the subject has no detectable neoplasia but is at high risk for disease recurrence. In embodiments, the cancer is selected from the group consisting of: adrenal, bladder, breast, cervical, colorectal, glioblasoma, head and neck, kidney chromophobe, kidney clear cell, kidney papillary, liver, lung adenocarcinoma, lung squamous, ovarian, pancreatic, melanoma, stomach, uterine corpus endometrial, and uterine carcinosarcoma. In embodiments, the cancer is selected from the group consisting of: prostate cancer, bladder, lung squamous, NSCLC, breast, head and neck, lung adenocarcinoma, GBM, Glioma, CML, AML, supretentorial ependyomas, acute promyelocytic leukemia, solitary fibrous tumors, and crizotinib resistant cancer. In embodiments, the cancer is selected from the group consisting of: CRC, head and neck, stomach, lung squamous, lung adenocarcinoma, prostate, bladder, stomach, renal cell carcinoma, and uterine. In embodiments, the cancer is selected from the group consisting of: melanoma, lung squamous, DLBCL, uterine, head and neck, uterine, liver, and CRC. In embodiments, the cancer is selected from the group consisting of: lymphoid cancer; Burkitt lymphoma, neuroblastoma, prostate adenocarcinoma, colorectal adenocarcinoma; Uterine/Endometrium Adenocarcinoma; MSI+; endometrium serous carcinoma; endometrium carcinosarcoma-malignant mesodermal mixed tumour; glioma; astrocytoma; GBM, acute myeloid leukemia associated with MDS; chronic lymphocytic leukemia-small lymphocytic lymphoma; myelodysplastic syndrome; acute myeloid leukemia; luminal NS carcinoma of breast; chronic myeloid leukemia; ductal carcinoma of pancreas; chronic myelomonocytic leukemia; myelofibrosis; myelodysplastic syndrome; prostate adenocarcinoma; essential thrombocythaemia; and medullomyoblastoma. In embodiments, the cancer is selected from the group consisting of: colorectal, uterine, endometrial, and stomach. In embodiments, the cancer is selected from the group consisting of: cervical, head and neck, anal, stomach, Burkitt's lymphoma, and nasopharyngeal carcinoma. In embodiments, the cancer is selected from the group consisting of: bladder, colorectal, and stomach. In embodiments, the cancer is selected from the group consisting of: lung, CRC, melanoma, breast, NSCLC, and CLL. In embodiments, the subject is a partial or non-responder to checkpoint inhibitor therapy. In embodiments, the subject is a partial or non-responder to CD40 agonist therapy, In embodiments, the cancer is selected from the group consisting of: bladder urothelial carcinoma (BLCA), breast invasive carcinoma (BRCA), breast cancer, cervical squamous cell carcinoma and endocervical adenocarcinoma (CESC), chronic lymphocytic leukemia (CLL), colorectal cancer (CRC), glioblastoma multiforme (GBM), head and neck squamous cell carcinoma (HNSC), kidney renal papillary cell carcinoma (KIRP), liver hepatocellular carcinoma (LIHC), lung adenocarcinoma (LUAD), lung squamous cell carcinoma (LUSC), pancreatic adenocarcinoma (PAAD), Prostate Cancer, skin cutaneous melanoma (SKCM), stomach adenocarcinoma (STAD), thyroid adenocarcinoma (THCA), and uterine corpus endometrioid carcinoma (UCEC). In embodiments, the cancer is selected from the group consisting of: colorectal cancer, uterine cancer, endometrium cancer, stomach cancer, and Lynch syndrome. In embodiments, the cancer is an MSI+ cancer.

### V. Pharmaceutical Compositions/Methods of Delivery

The present invention is also directed to pharmaceutical compositions comprising an effective amount of one or more compounds according to the present invention (including a pharmaceutically acceptable salt, thereof), optionally in combination with a pharmaceutically acceptable carrier, excipient or additive.

When administered as a combination, the therapeutic agents (i.e. the neoplasia vaccine or immunogenic composition and one or more inhibitors, such as one or more checkpoint inhibitors or CD40 agonists) can be formulated as separate compositions that are given at the same time or different times, or the therapeutic agents can be given as a single composition.

The compositions may be administered once daily, twice daily, once every two days, once every three days, once every four days, once every five days, once every six days, once every seven days, once every two weeks, once every three weeks, once every four weeks, once every two months, once every six months, or once per year. The dosing interval can be adjusted according to the needs of individual patients. For longer intervals of administration, extended release or depot formulations can be used.

The compositions of the invention can be used to treat diseases and disease conditions that are acute, and may also be used for treatment of chronic conditions. In particular, the compositions of the invention are used in methods to treat or prevent a neoplasia.

In certain embodiments, the compounds of the invention are administered for time periods exceeding two weeks, three weeks, one month, two months, three months, four months, five months, six months, one year, two years, three years, four years, or five years, ten years, or fifteen years; or for example, any time period range in days, months or years in which the low end of the range is any time period between 14 days and 15 years and the upper end of the range is between 15 days and 20 years (e.g., 4 weeks and 15 years, 6 months and 20 years). In some cases, it may be advantageous for the compounds of the invention to be administered for the remainder of the patient's life. In some embodiments, the patient is monitored to check the progression of the disease or disorder, and the dose is adjusted accordingly. In some embodiments, treatment according to the invention is effective for at least two weeks, three weeks, one month, two months, three months, four months, five months, six months, one year, two years, three years, four years, or five years, ten years, fifteen years, twenty years, or for the remainder of the subject's life.

As described herein, in certain embodiments, administration of the inhibitor is initiated before initiation of administration of the neoplasia vaccine or immunogenic composition. In other embodiments, administration of the inhibitor is initiated after initiation of administration of the neoplasia vaccine or immunogenic composition. In still other embodiments, administration of the inhibitor is initiated simultaneously with the initiation of administration of the neoplasia vaccine or immunogenic composition,

Administration of the inhibitor, such as a checkpoint inhibitor or CD40 agonist, may continue every 2, 3, 4, 5, 6, 7, 8 or more weeks after the first administration of the inhibitor, such as a checkpoint inhibitor or CD40 agonist. It is understood that week 1 is meant to include days 1-7, week 2 is meant to include days 8-14, week 3 is meant to include days 15-21 and week 4 is meant to include days 22-28. When dosing is described as being on weekly intervals it means approximately 7 days apart although in any given week the day can be one or more days before or after the scheduled day.

In combination therapies, one or more inhibitors, such as a checkpoint inhibitors or CD40 agonists, may be administered to the patient throughout the treatment regimen. For instance, if a treatment regimen lasts for 52 weeks, one or more checkpoint inhibitors such as Nivolumab, or Ipilimumab, or a CD40 agonist such as APX005M may be administered throughout the 52 weeks. In some embodiments, one inhibitor, such as a checkpoint inhibitor or CD40 agonist, may be administered for the length of the treatment regimen whereas one or more other inhibitors, such as a checkpoint inhibitor or CD40 agonist, which are part of the combination therapy may be administered for a shorter time period.

In certain embodiments, administration of the inhibitor, such as a checkpoint inhibitor or CD40 agonist, is withheld during the week prior to administration of the neoplasia vaccine or immunogenic composition. In other embodiments, administration of the inhibitor, such as a checkpoint inhibitor or CD40 agonist, is withheld during administration of the neoplasia vaccine or immunogenic composition.

Surgical resection uses surgery to remove abnormal tissue in cancer, such as mediastinal, neurogenic, or germ cell tumors, or thymoma. In certain embodiments, administration of the inhibitor, such as a checkpoint inhibitor or CD40 agonist, is initiated following tumor resection. In other embodiments, administration of the neoplasia vaccine or immunogenic composition is initiated 1, 2, 3, 4, 5, 6, 7, 8, 9, 10, 11, 12, 13, 14, 15 or more weeks after tumor resection. In some embodiments, administration of the neoplasia vaccine or immunogenic composition is initiated 4, 5, 6, 7, 8, 9, 10, 11 or 12 weeks after tumor resection.

Prime/boost regimens refer to the successive administrations of a vaccine or immunogenic or immunological compositions. In certain embodiments, administration of the neoplasia vaccine or immunogenic composition is in a prime/boost dosing regimen, for example administration of the neoplasia vaccine or immunogenic composition at weeks 1, 2, 3 or 4 as a prime and administration of the neoplasia vaccine or immunogenic composition is at months 2, 3 or 4 as a boost. In another embodiment heterologous prime-boost strategies are used to elicit a greater cytotoxic T-cell response (see Schneider et al., Induction of CD8+ T cells using heterologous prime-boost immunization strategies, Immunological Reviews Volume 170, Issue 1, pages 29-38, August 1999). In another embodiment DNA encoding neoantigens is used to prime followed by a protein boost. In another embodiment protein is used to prime followed by boosting with a virus encoding the neoantigen. In another embodiment a virus encoding the neoantigen is used to prime and another virus is used to boost. In another embodiment protein is used to prime and DNA is used to boost. In some embodiments, a DNA vaccine or immunogenic composition is used to prime a T-cell response and a recombinant viral vaccine or immunogenic composition is used to boost the response. In some embodiments, a viral vaccine or immunogenic composition is co-administered with a protein or DNA vaccine or immunogenic composition to act as an adjuvant for the protein or DNA vaccine or immunogenic composition. The patient can then be boosted with either the viral vaccine or immunogenic composition, protein, or DNA vaccine or immunogenic composition (see Hutchings et al., Combination of protein and viral vaccines induces potent cellular and humoral immune responses and enhanced protection from murine malaria challenge. Infect Immun, 2007 December; 75(12):5S19-26. Epub 2007 October 1).

As used herein, the term “fixed intermittent dosing regimen” refers to repeating cycles of preplanned drug administration in which the drug is administered on one or more consecutive days (“days on”) followed by one or more consecutive days of rest on which the drug is not administered (“days off”).

In some embodiments, the cycles are regular, in that the pattern of days on and days off is the same in each cycle. In some embodiments, the cycles are irregular, in that the pattern of days on and days off differs from one cycle to the next cycle. In some embodiments, each of the repeating cycles, however, is preplanned in that it is not determined solely in response to the appearance of one or more adverse events. In some embodiments, administration of the composition comprising the first component and/or the second component is repeated for one to ten cycles, such as for example one cycle, two cycles, three cycles, four cycles, five cycles, six cycles, seven cycles, eight cycles, nine cycles or ten cycles.

In some embodiments, a cycle comprises 3 days to 60 days. In some embodiments, a cycle comprises 7 to 50 days, such as 7 to 30 days, 7 to 21 days, or 7 to 14 days. In some embodiments, a cycle consists of 7 days.

In some embodiments, the fixed intermittent dosing regimen comprises a repeating cycle of administration of an effective amount of said composition comprising the first component and/or the second component on 1 to 5 consecutive days, such as 2 to 5 consecutive days, followed by 6 to 2 days of rest, such as 5 to 2 days of rest. In some embodiments, the fixed intermittent dosing regimen comprises a repeating cycle of administration of an effective amount of said composition comprising the first component and/or the second component on 5 consecutive days followed by 2 days of rest. In some embodiments, the fixed intermittent dosing regimen comprises a repeating cycle of administration of an effective amount of said composition comprising the first component and/or the second component on 4 consecutive days followed by 3 days of rest. In some embodiments, the fixed intermittent dosing regimen comprises a repeating cycle of administration of an effective amount of said composition comprising the first component and/or the second component on 3 consecutive days followed by 4 days of rest.

In some embodiments, the fixed intermittent dosing regimen comprises a repeating cycle of administration of an effective amount of said composition comprising the first component and/or the second component on 1 to 5 consecutive days, such as 2 to 5 consecutive days, followed by 6 to 2 days of rest, such as 5 to 2 days of rest. In some embodiments, placebo is administered on said days of rest.

The pharmaceutical compositions can be processed in accordance with conventional methods of pharmacy to produce medicinal agents for administration to patients in need thereof, including humans and other mammals.

Modifications of the neoantigenic peptides can affect the solubility, bioavailability and rate of metabolism of the peptides, thus providing control over the delivery of the active species. Solubility can be assessed by preparing the neoantigenic peptide and testing according to known methods well within the routine practitioner's skill in the art.

It has been found that a pharmaceutical composition comprising succinic acid or a pharmaceutically acceptable salt thereof (succinate) can provide improved solubility for the neoantigenic peptides. Thus, in one aspect, the invention provides a pharmaceutical composition comprising: at least one neoantigenic peptide or a pharmaceutically acceptable salt thereof; a pH modifier (such as a base, such as a dicarboxylate or tricarboxylate salt, for example, a pharmaceutically acceptable salt of succinic acid or citric acid); and a pharmaceutically acceptable carrier. Such pharmaceutical compositions can be prepared by combining a solution comprising at least one neoantigenic peptide with a base, such as a dicarboxylate or tricarboxylate salt, such as a pharmaceutically acceptable salt of succinic acid or citric acid (such as sodium succinate), or by combining a solution comprising at least one neoantigenic peptide with a solution comprising a base, such as a dicarboxylate or tricarboxylate salt, such as a pharmaceutically acceptable salt of succinic acid or citric acid (including, e.g., a succinate buffer solution). In certain embodiments, the pharmaceutical composition comprises sodium succinate. In certain embodiments, the pH modifier (such as citrate or succinate) is present in the composition at a concentration from about 1 mM to about 10 mM, and, in certain embodiments, at a concentration from about 1.5 mM to about 7.5 mM, or about 2.0 to about 6.0 mM, or about 3.75 to about 5.0 mM.

In certain embodiments of the pharmaceutical composition the pharmaceutically acceptable carrier comprises water. In certain embodiments, the pharmaceutically acceptable carrier further comprises dextrose. In certain embodiments, the pharmaceutically acceptable earner further comprises dimethylsulfoxide. In certain embodiments, the pharmaceutical composition further comprises an immunomodulator or adjuvant. In certain embodiments, the immunomodulator or adjuvant is selected from the group consisting of poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila's QS21 stimulon. In certain embodiments, the immunomodulator or adjuvant comprises poly-ICLC,

Xanthenone derivatives such as, for example, Vadimezan or AsA404 (also known as 5,6-dimethylaxanthenone-4-acetic acid (DMXAA)), may also be used as adjuvants according to embodiments of the invention. Alternatively, such derivatives may also be administered in parallel to the vaccine or immunogenic composition of the invention, for example via systemic or intratumoral delivery, to stimulate immunity at the tumor site. Without being bound by theory, it is believed that such xanthenone derivatives act by stimulating interferon (IFN) production via the stimulator of IFN gene ISTING) receptor (see e.g., Conlon et al. (2013) Mouse, but not Human STING, Binds and Signals in Response to the Vascular Disrupting Agent 5,6-Di-methylxanthenone-4-Acetic Acid, journal of Immunology, 190:5216-25 and Kim et al. (2013) Anticancer Flavonoids are Mouse-Selective STING Agonists, 8: 1396-1401).

The vaccine or immunological composition may also include an adjuvant compound chosen from the acrylic or methacrylic polymers and the copolymers of maleic anhydride and an alkenyl derivative. It is in particular a polymer of acrylic or methacrylic acid cross-linked with a polyalkenyl ether of a sugar or polyalcohol (carbomer), in particular cross-linked with an allyl sucrose or with allylpentaerythritol. It may also be a copolymer of maleic anhydride and ethylene cross-linked, for example, with divinyl ether (see U.S. Pat. No. 6,713,068 hereby incorporated by reference in its entirety).

In certain embodiments, the pH modifier can stabilize the adjuvant or immunomodulator as described herein.

In certain embodiments, a pharmaceutical composition comprises: one to five peptides, dimethyl sulfoxide (DMSO), dextrose, water, succinate, poly I: poly C, poly-L-lysine, carboxymethylcellulose, and chloride. In certain embodiments, each of the one to five peptides is present at a concentration between 200 μg/ml and 500 μg/ml, 300-400 μg/ml. In certain embodiments, the pharmaceutical composition comprises ≤3% DMSO by volume, about 4-5% DMSO. In certain embodiments, the pharmaceutical composition comprises 3.5-5.5% dextrose, 4.9-5.0% dextrose in water. In certain embodiments, the pharmaceutical composition comprises ≤5.0 mM succinate, 3.6-3.7 mM succinate (e.g., as sodium succinate). In certain embodiments, the pharmaceutical composition comprises ≥0.4 mg/ml poly I: poly C, for example, 1.0-2.2 mg/ml, for example, 1.7-1.9 mg/ml. In certain embodiments, the pharmaceutical composition comprises ≥0.375 mg/ml poly-L-Lysine, 0.5-2.0 mg/ml, or 1.5 mg/ml. In certain embodiments, the pharmaceutical composition comprises ≥1.25 mg/ml sodium carboxymethylcellulose, 2-7 mg/ml, for example, 4-5 mg/ml. In certain embodiments, the pharmaceutical composition comprises ≥0.225% sodium chloride, 0.5-1.0% sodium chloride, or 0.8-2.0% sodium chloride.

Pharmaceutical compositions comprise the herein-described tumor specific neoantigenic peptides in a therapeutically effective amount for treating diseases and conditions (e.g., a neoplasia/tumor), which have been described herein, optionally in combination with a pharmaceutically acceptable additive, carrier and/or excipient. One of ordinary skill in the art from this disclosure and the knowledge in the art will recognize that a therapeutically effective amount of one of more compounds according to the present invention may vary with the condition to be treated, its severity, the treatment regimen to be employed, the pharmacokinetics of the agent used, as well as the patient (animal or human) treated.

To prepare the pharmaceutical compositions according to the present invention, a therapeutically effective amount of one or more of the compounds according to the present invention can be intimately admixed with a pharmaceutically acceptable carrier according to conventional pharmaceutical compounding techniques to produce a dose. A carrier may take a wide variety of forms depending on the form of preparation desired for administration, e.g., ocular, oral, topical or parenteral, including gels, creams ointments, lotions and time released implantable preparations, among numerous others. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated or sustained release by standard techniques.

The active compound is included in the pharmaceutically acceptable earner or diluent in an amount sufficient to deliver to a patient a therapeutically effective amount for the desired indication, without causing serious toxic effects in the patient treated,

Oral compositions generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid or corn starch; a lubricant such as magnesium stearate; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material herein discussed, a liquid carrier such as fatty oil. In addition, dosage unit forms can contain various other materials which modify the physical form of the dosage unit, for example, coatings of sugar, shellac, or enteric agents.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein.

Methods of formulating such slow or controlled release compositions of pharmaceutically active ingredients, are known in the art and described in several issued US patents, some of which include, but are not limited to, U.S. Pat. Nos. 3,870,790; 4,226,859; 4,369,172: 4,842,866 and 5,705,190, the disclosures of which are incorporated herein by reference in their entireties. Coatings can be used for delivery of compounds to the intestine (see, e.g., U.S. Pat. Nos. 6,638,534, 5,541,171, 5,217,720, and 6,569,457, and references cited therein).

The active compound or pharmaceutically acceptable salt thereof may also be administered as a component of an elixir, suspension, syrup, wafer, chewing gum or the like. A syrup may contain, in addition to the active compounds, sucrose or fructose as a sweetening agent and certain preservatives, dyes and colorings and flavors,

Solutions or suspensions used for ocular, parenteral, intradermal, subcutaneous, or topical application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose.

In certain embodiments, the pharmaceutically acceptable carrier is an aqueous solvent, i.e., a solvent comprising water, optionally with additional co-solvents. Exemplary pharmaceutically acceptable carriers include water, buffer solutions in water (such as phosphate-buffered saline (PBS), and 5% dextrose in water (D5W). In certain embodiments, the aqueous solvent further comprises dimethyl sulfoxide (DMSO), e.g., in an amount of about 1-4%, or 2-3%. In certain embodiments, the pharmaceutically acceptable carrier is isotonic (i.e., has substantially the same osmotic pressure as a body fluid such as plasma).

In one embodiment, the active compounds are prepared with carriers that protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid, and polylactic-co-glycolic acid (PLGA). Methods for preparation of such formulations are within the ambit of the skilled artisan in view of this disclosure and the knowledge in the art.

A skilled artisan from this disclosure and the knowledge in the art recognizes that in addition to tablets, other dosage forms can be formulated to provide slow or controlled release of the active ingredient. Such dosage forms include, but are not limited to, capsules, granulations and gel-caps.

Liposomal suspensions may also be pharmaceutically acceptable carriers. These may be prepared according to methods known to those skilled in the art. For example, liposomal formulations may be prepared by dissolving appropriate lipid(s) in an inorganic solvent that is then evaporated, leaving behind a thin film of dried lipid on the surface of the container. An aqueous solution of the active compound can then introduced into the container. The container is then swirled by hand to free lipid material from the sides of the container and to disperse lipid aggregates, thereby forming the liposomal suspension. Other methods of preparation well known by those of ordinary skill may also be used in this aspect of the present invention,

The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid earners or both, and then, if necessary, shaping the product.

Formulations and compositions suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. A topical delivery system that can be used includes is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which stuff is administered, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing in addition to the active ingredient such carriers as are known in the art to be appropriate.

The parenteral preparation can be enclosed in ampoules, disposable syringes or multiple dose vials made of glass or plastic. If administered intravenously, carriers can include, for example, physiological saline or phosphate buffered saline (PBS).

For parenteral formulations, the carrier usually comprises sterile water or aqueous sodium chloride solution, though other ingredients including those which aid dispersion may be included. Of course, where sterile water is to be used and maintained as sterile, the compositions and carriers are also sterilized. Injectable suspensions may also be prepared, in which case appropriate liquid carriers, suspending agents and the like may be employed.

Formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

Administration of the active compound may range from continuous (intravenous drip) to several oral administrations per day (for example, Q.I.D.) and may include oral, topical, eye or ocular, parenteral, intramuscular, intravenous, sub-cutaneous, transdermal (which may include a penetration enhancement agent), buccal and suppository administration, among other routes of administration, including through an eye or ocular route.

The neoplasia vaccine or immunogenic composition and the at least one inhibitor, such as a checkpoint inhibitor or CD40 agonist, and any additional agents, may be administered by injection, orally, parenterally, by inhalation spray, rectally, vaginally, or topically in dosage unit formulations containing conventional pharmaceutically acceptable carriers, adjuvants, and vehicles. The term parenteral as used herein includes, into a lymph node or nodes, subcutaneous, intravenous, intramuscular, intrasternal, infusion techniques, intraperitoneally, eye or ocular, intravitreal, intrabuccal, transdermal, intranasal, into the brain, including intracranial and intradural, into the joints, including ankles, knees, hips, shoulders, elbows, wrists, directly into tumors, and the like, and in suppository form.

In certain embodiments, the vaccine or immunogenic composition or the one of more inhibitors, such as checkpoint inhibitors or CD40 agonist, are administered intravenously or subcutaneously.

Application of the subject therapeutics may be local, so as to be administered at the site of interest. In certain embodiments, the inhibitor, such as a checkpoint inhibitor or CD40 agonist, is administered subcutaneously near the site of administration of the neoplasia “vaccine or immunogenic composition, for example within about 1, 2, 3, 4, 5, 6, 7, 8, 9, 10 cm of the site of vaccine or immunogenic composition administration, and, for example, within 5 cm of the site of administration of the neoplasia vaccine or immunogenic composition. It is to be understood by one skilled in the art administering the compositions that the concentration of the inhibitor, such as a checkpoint inhibitor or CD40 agonist, administered to the subject may be changed based on the location of administration. For example, if the inhibitor, such as a checkpoint inhibitor or CD40 agonist, is administered near the site of administration of the neoplasia vaccine or immunogenic composition, then the concentration of the inhibitor may be decreased.

Various techniques can be used for providing the subject compositions at the site of interest, such as injection, use of catheters, trocars, projectiles, pluronic gel, stents, sustained drag release polymers or other device which provides for internal access. Where an organ or tissue is accessible because of removal from the patient, such organ or tissue may be bathed in a medium containing the subject compositions, the subject compositions may be painted onto the organ, or may be applied in any convenient way.

The tumor specific neoantigenic peptides may be administered through a device suitable for the controlled and sustained release of a composition effective in obtaining a desired local or systemic physiological or pharmacological effect. The method includes positioning the sustained released drug delivery system at an area wherein release of the agent is desired and allowing the agent to pass through the device to the desired area of treatment.

The tumor specific neoantigenic peptides may be utilized in combination with at least one known other therapeutic agent, or a pharmaceutically acceptable salt of said agent. Examples of known therapeutic agents which can be used for combination therapy include, but are not limited to, corticosteroids (e.g., cortisone, prednisone, dexamethasone), non-steroidal anti-inflammatory drugs (NSAIDS) (e.g., ibuprofen, celecoxib, aspirin, indomethicin, naproxen), alkylating agents such as busulfan, cis-platin, mitomycin C, and carboplatin; antimitotic agents such as colchicine, vinblastine, paclitaxel, and docetaxel; topo I inhibitors such as camptothecin and topotecan; topo II inhibitors such as doxorubicin and etoposide; and/or RNA/DNA antimetabolites such as 5-azacytidine, 5-fluorouracil and methotrexate; DNA antimetabolites such as 5-fluoro-2′-deoxy-uridine, ara-C, hydroxyurea and thioguanine; antibodies such as BERCEPT1N and RITUXAN.

In certain embodiments, administration of the compositions described herein may be combined with the administration of antagonists that block the release of histamine and anti-inflammatory drugs to prevent adverse allergic reactions. H1 and H2 antagonists may be administered to the patient before the administration of the compositions described herein.

It should be understood that in addition to the ingredients particularly mentioned herein, the formulations of the present invention may include other agents conventional in the art having regard to the type of formulation in question, for example, those suitable for oral administration may include flavoring agents.

Pharmaceutically acceptable salt forms may be the chemical form of compounds according to the present invention for inclusion in pharmaceutical compositions according to the present invention,

The present compounds or their derivatives, including prodrug forms of these agents, can be provided in the form of pharmaceutically acceptable salts. As used herein, the term pharmaceutically acceptable salts or complexes refers to appropriate salts or complexes of the active compounds according to the present invention which retain the desired biological activity of the parent compound and exhibit limited toxicological effects to normal cells. Non-limiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, and polyglutamic acid, among others; (b) base addition salts formed with metal cations such as zinc, calcium, sodium, potassium, and the like, among numerous others.

The compounds herein are commercially available or can be synthesized. As can be appreciated by the skilled artisan, further methods of synthesizing the compounds of the formulae herein is evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2nd. Ed., Wiley-VCH Publishers (1999); T. W. Greene and P. G. M. Wuts, Protective Groups in Organic Synthesis, 3rd. Ed., John Wiley and Sons (1999); L. Fieser and M. Fieser, Fieser and Fieser's Reagents for Organic Synthesis, John Wiley and Sons (1999); and L. Paquette, ed., Encyclopedia of Reagents for Organic Synthesis, John Wiley and Sons (1995), and subsequent editions thereof.

### VI. Dosage

When the agents described herein are administered as pharmaceuticals to humans or animals, they can be given per se or as a pharmaceutical composition containing active ingredient in combination with a pharmaceutically acceptable carrier, excipient, or diluent.

Actual dosage levels and time course of administration of the active ingredients in the pharmaceutical compositions of the invention can be varied so as to obtain an amount of the active ingredient which is effective to achieve the desired therapeutic response for a particular patient, composition, and mode of administration, without being toxic to the patient. Generally, agents or pharmaceutical compositions of the invention are administered in an amount sufficient to reduce or eliminate symptoms associated with viral infection and/or autoimmune disease.

A dose of an agent can be the maximum that a patient can tolerate and not develop serious or unacceptable side effects.

Determination of an effective amount is well within the capability of those skilled in the art, especially in light of the detailed disclosure provided herein. Generally, an efficacious or effective amount of an agent is determined by first administering a low dose of the agent(s) and then incrementally increasing the administered dose or dosages until a desired effect (e.g., reduce or eliminate symptoms associated with viral infection or autoimmune disease) is observed in the treated subject, with minimal or acceptable toxic side effects. Applicable methods for determining an appropriate dose and dosing schedule for administration of a pharmaceutical composition of the present invention are described, for example, in Goodman and Oilman's The Pharmacological Basis of Therapeutics, Goodman et al., eds., 11th Edition, McGraw-Hill 2005, and Remington: The Science and Practice of Pharmacy, 20th and 21st Editions, Gennaro and University of the Sciences in Philadelphia, Eds., Lippencott Williams & Wilkins (2003 and 2005), each of which is hereby incorporated by reference.

Unit dosage formulations can be those containing a daily dose or unit, daily sub-dose, as herein discussed, or an appropriate fraction thereof, of the administered ingredient.

The dosage regimen for treating a disorder or a disease with the tumor specific neoantigenic peptides of this invention and/or compositions of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods.

The amounts and dosage regimens administered to a subject can depend on a number of factors, such as the mode of administration, the nature of the condition being treated, the body weight of the subject being treated and the judgment of the prescribing physician; all such factors being within the ambit of the skilled artisan from this disclosure and the knowledge in the art. In some embodiments, an initial series of closely spaced immunizations may be administered to induce an immune response followed by a period of rest to allow memory T cells to be established and booster immunizations to expand the response. Alternatively, the priming doses may be administered over a longer period of time, and boosts may be administered more frequently for a longer period. For instance, a long priming period may be followed by boosts administered every 2 months for 1 year.

The amount of compound included within therapeutically active formulations according to the present invention is an effective amount for treating the disease or condition.

In general, a therapeutically effective amount of a compound in dosage form can range from slightly less than about 0.025 mg/kg/day to about 2.5 g/kg/day, about 0.1 mg/kg/day to about 100 mg/kg/day of the patient or considerably more, depending upon the compound used, the condition or infection treated and the route of administration, although exceptions to this dosage range may be contemplated by the present invention. In some embodiments, compounds according to the present invention are administered in amounts ranging from about 1 mg/kg/day to about 100 mg/kg/day. The dosage of the compound can depend on the condition being treated, the particular compound, and other clinical factors such as weight and condition of the patient and the route of administration of the compound. It is to be understood that the present invention has application for both human and veterinary use.

According to certain exemplary embodiments, the vaccine or immunogenic composition is administered at a dose of about 10 μg-1 mg per neoantigenic peptide. According to certain exemplary embodiments, the vaccine or immunogenic composition is administered at an average weekly dose level of about 10 μg-2000 μg per neoantigenic peptide. In some cases, a single dose of one or more neoantigenic peptides has a concentration between 100 μg/ml to 1000 μg/ml, 300-600 μg/ml, or 400-500 μg/ml. According to certain exemplary embodiments, the inhibitor, such as a checkpoint inhibitor or CD40 agonist, is administered at a dose of about 0.1-10 mg/kg. According to certain exemplary embodiments, the anti-CTLA4 antibody ipilimumab is administered at a dose of about 1 mg kg-3 mg/kg. For example, in certain exemplary embodiments, Nivolumab is given dosing at the standard single agent dosing level of 3 mg/kg. According to certain exemplary embodiments, the anti-CD40 antibody APX500M is administered at a dose of about 0.05 mg kg-10 mg/kg, for example, 0.1 mg/kg-2 mg/kg. Inhibitors, such as a checkpoint inhibitor or CD40 agonist, may be diluted before administration to a subject. When the one or more inhibitors, such as a checkpoint inhibitor or CD40 agonist, are administered at the site of administration of the vaccine or immunogenic composition, the inhibitor can be administered at a dose of about 0.1-1 mg per site of administration of the neoplasia vaccine or immunogenic composition. When neoantigens and an inhibitor, such as a checkpoint inhibitor or CD40 agonist, are being administered on the same day, neoantigens may be administered before the inhibitors, such as a checkpoint inhibitor or CD40 agonist. Alternatively, neoantigens may be administered last to the subject.

In some embodiments, a subject is administered neoantigenic peptides at a dosage of 10 μg to 2,000 μg per peptide. In some embodiments, a subject is administered neoantigenic peptides at a dosage of at least 10 μg, 50 μg, 100 μg, 150 μg, 200 μg, 250 μg, 300 μg, 400 μg, 500 μg, 600 μg, 800 μg, 1000 μg or 1500 μg per peptide. In some embodiments, a subject is administered neoantigenic peptides at a dosage of at most 2,000 μg, 1500 μg, 1000 μg, 800 μg, 700 μg, 600 μg, 500 μg, 400 μg, 300 μg, 250 μg, 200 μg, 100 μg, 75 μg per peptide. In some embodiments, a subject is administered neoantigenic peptides at a dosage of 10 μg to 50 μg, 10 μg to 100 μg, 10 μg to 200 μg, 10 μg to 300 μg, 10 μg to 400 μg, 10 μg to 500 μg, 10 μg to 600 μg, 10 μg to 800 μg, 10 μg to 1,000 μg, 10 μg to 1,500 μg, 10 μg to 2,000 μg, 50 μg to 100 μg, 50 μg to 200 μg, 50 μg to 300 μg, 50 μg to 400 μg, 50 μg to 500 μg, 50 μg to 600 μg, 50 μg to 800 μg, 50 μg to 1,000 μg, 50 μg to 1,500 μg, 50 μg to 2,000 μg, 100 μg to 200 μg, 100 μg to 300 μg, 100 μg to 400 μg, 100 μg to 500 μg, 100 μg to 600 μg, 100 μg to 800 μg, 100 μg to 1,000 μg, 100 μg to 1,500 μg, 100 μg to 2,000 μg, 200 μg to 300 μg, 200 μg to 400 μg, 200 μg to 500 μg, 200 μg to 600 μg, 200 μg to 800 μg, 200 μg to 1,000 μg, 200 μg to 1,500 μg, 200 μg to 2,000 μg, 300 μg to 400 μg, 300 μg to 500 μg, 300 μg to 600 μg, 300 μg to 800 μg, 300 μg to 1,000 μg, 300 μg to 1,500 μg, 300 μg to 2,000 μg, 400 μg to 500 μg, 400 μg to 600 μg, 400 μg to 800 μg, 400 μg to 1,000 μg, 400 μg to 1,500 μg, 400 μg to 2,000 μg, 500 μg to 600 μg, 500 μg to 800 μg, 500 μg to 1,000 μg, 500 μg to 1,500 μg, 500 μg to 2,000 μg, 600 μg to 800 μg, 600 μg to 1,000 μg, 600 μg to 1,500 μg, 600 μg to 2,000 μg, 800 μg to 1,000 μg, 800 μg to 1,500 μg, 800 μg to 2,000 μg, 1,000 μg to 1,500 μg, 1,000 μg to 2,000 μg, or 1,500 μg to 2,000 μg per peptide. In some embodiments, a subject is administered neoantigenic peptides at a dosage of 10 μg, 50 μg, 100 μg, 200 μg, 300 μg, 400 μg, 500 μg, 600 μg, 800 μg, 1,000 μg, 1,500 μg, or 2,000 μg per peptide.

In some embodiments, a subject is administered nivolumab at a dosage of 50 mg to 400 mg. In some embodiments, a subject is administered nivolumab at a dosage of at least 50 mg, 75 mg, 100 mg, 150 mg, 200 mg, 220 mg, 240 mg, 250 mg, 260 mg, 270 mg, 280 mg, 300 mg, 320 mg or 350 mg. In some embodiments, a subject is administered nivolumab at a dosage of at most 400 mg, 350 mg, 300 mg, 260 mg, 240 mg, 200 mg, 150 mg, 100 mg or 75 mg. In some embodiments, a subject is administered nivolumab at a dosage of 50 mg to 75 mg, 50 mg to 100 mg, 50 mg to 150 mg, 50 mg to 200 mg, 50 mg to 220 mg, 50 mg to 240 mg, 50 mg to 260 mg, 50 mg to 280 mg, 50 mg to 300 mg, 50 mg to 350 mg, 50 mg to 400 mg, 75 mg to 100 mg, 75 mg to 150 mg, 75 mg to 200 mg, 75 mg to 220 mg, 75 mg to 240 mg, 75 mg to 260 mg, 75 mg to 280 mg, 75 mg to 300 mg, 75 mg to 350 mg, 75 mg to 400 mg, 100 mg to 150 mg, 100 mg to 200 mg, 100 mg to 220 mg, 100 mg to 240 mg, 100 mg to 260 mg, 100 mg to 280 mg, 100 mg to 300 mg, 100 mg to 350 mg, 100 mg to 400 mg, 150 mg to 200 mg, 150 mg to 220 mg, 150 mg to 240 mg, 150 mg to 260 mg, 150 mg to 280 mg, 150 mg to 300 mg, 150 mg to 350 mg, 150 mg to 400 mg, 200 mg to 220 mg, 200 mg to 240 mg, 200 mg to 260 mg, 200 mg to 280 mg, 200 mg to 300 mg, 200 mg to 350 mg, 200 mg to 400 mg, 220 mg to 240 mg, 220 mg to 260 mg, 220 mg to 280 mg, 220 mg to 300 mg, 220 mg to 350 mg, 220 mg to 400 mg, 240 mg to 260 mg, 240 mg to 280 mg, 240 mg to 300 mg, 240 mg to 350 mg, 240 mg to 400 mg, 260 mg to 280 mg, 260 mg to 300 mg, 260 mg to 350 mg, 260 mg to 400 mg, 280 mg to 300 mg, 280 mg to 350 mg, 280 mg to 400 mg, 300 mg to 350 mg, 300 mg to 400 mg, or 350 mg to 400 mg. In some embodiments, a subject is administered nivolumab at a dosage of 50 mg, 75 mg, 100 mg, 150 mg, 200 mg, 220 mg, 240 mg, 260 mg, 280 mg, 300 mg, 350 mg, or 400 mg.

In some embodiments, a subject is administered ipilimumab at a dosage of 0.001 mg/kg to 3.5 mg/kg. In some embodiments, a subject is administered ipilimumab at a dosage of at least 0.001 mg/kg, 0.005 mg/kg, 0.01 mg/kg, 0.05 mg/kg, 0.1 mg/kg, 0.5 mg/kg, 1.0 mg/kg 1.5 mg/kg, 2.0 mg/kg, 2.5 mg/kg or 3 mg/kg. In some embodiments, a subject is administered ipilimumab at a dosage of at most 3.5 mg/kg, 3.0 mg/kg, 2.5 mg/kg, 2.0 mg/kg, 1.5 mg/kg, 1.0 mg/kg, 0.5 mg/kg, 0.1 mg/kg, 0.05 mg/kg or 0.01 mg/kg. In some embodiments, a subject is administered ipilimumab at a dosage of 0.001 mg/kg to 0.005 mg/kg, 0.001 mg/kg to 0.01 mg/kg, 0.001 mg/kg to 0.05 mg/kg, 0.001 mg/kg to 0.1 mg/kg, 0.001 mg/kg to 0.5 mg/kg, 0.001 mg/kg to 1 mg/kg, 0.001 mg/kg to 1.5 mg/kg, 0.001 mg/kg to 2 mg/kg, 0.001 mg/kg to 2.5 mg/kg, 0.001 mg/kg to 3 mg/kg, 0.001 mg/kg to 3.5 mg/kg, 0.005 mg/kg to 0.01 mg/kg, 0.005 mg/kg to 0.05 mg/kg, 0.005 mg/kg to 0.1 mg/kg, 0.005 mg/kg to 0.5 mg/kg, 0.005 mg/kg to 1 mg/kg, 0.005 mg/kg to 1.5 mg/kg, 0.005 mg/kg to 2 mg/kg, 0.005 mg/kg to 2.5 mg/kg, 0.005 mg/kg to 3 mg/kg, 0.005 mg/kg to 3.5 mg/kg, 0.01 mg/kg to 0.05 mg/kg, 0.01 mg/kg to 0.1 mg/kg, 0.01 mg/kg to 0.5 mg/kg, 0.01 mg/kg to 1 mg/kg, 0.01 mg/kg to 1.5 mg/kg, 0.01 mg/kg to 2 mg/kg, 0.01 mg/kg to 2.5 mg/kg, 0.01 mg/kg to 3 mg/kg, 0.01 mg/kg to 3.5 mg/kg, 0.05 mg/kg to 0.1 mg/kg, 0.05 mg/kg to 0.5 mg/kg, 0.05 mg/kg to 1 mg/kg, 0.05 mg/kg to 1.5 mg/kg, 0.05 mg/kg to 2 mg/kg, 0.05 mg/kg to 2.5 mg/kg, 0.05 mg/kg to 3 mg/kg, 0.05 mg/kg to 3.5 mg/kg, 0.1 mg/kg to 0.5 mg/kg, 0.1 mg/kg to 1 mg/kg, 0.1 mg/kg to 1.5 mg/kg, 0.1 mg/kg to 2 mg/kg, 0.1 mg/kg to 2.5 mg/kg, 0.1 mg/kg to 3 mg/kg, 0.1 mg/kg to 3.5 mg/kg, 0.5 mg/kg to 1 mg/kg, 0.5 mg/kg to 1.5 mg/kg, 0.5 mg/kg to 2 mg/kg, 0.5 mg/kg to 2.5 mg/kg, 0.5 mg/kg to 3 mg/kg, 0.5 mg/kg to 3.5 mg/kg, 1 mg/kg to 1.5 mg/kg, 1 mg/kg to 2 mg/kg, 1 mg/kg to 2.5 mg/kg, 1 mg/kg to 3 mg/kg, 1 mg/kg to 3.5 mg/kg, 1.5 mg/kg to 2 mg/kg, 1.5 mg/kg to 2.5 mg/kg, 1.5 mg/kg to 3 mg/kg, 1.5 mg/kg to 3.5 mg/kg, 2 mg/kg to 2.5 mg/kg, 2 mg/kg to 3 mg/kg, 2 mg/kg to 3.5 mg/kg, 2.5 mg/kg to 3 mg/kg, 2.5 mg/kg to 3.5 mg/kg, or 3 mg/kg to 3.5 mg/kg. In some embodiments, a subject is administered ipilimumab at a dosage of 0.001 mg/kg, 0.005 mg/kg, 0.01 mg/kg, 0.05 mg/kg, 0.1 mg/kg, 0.5 mg/kg, 1 mg/kg, 1.5 mg/kg, 2 mg/kg, 2.5 mg/kg, 3 mg/kg, or 3.5 mg/kg.

APX005M is an IgG1 humanized version of a rabbit R-8 monoclonal antibody with the S267E mutation at the Fc region with a high affinity for CD40. The sequence, production and treatment options using the APX005M antibody have been described in detail in U.S. Pat. Nos. 8,778,345 and 9,676,861. SEQ ID Nos: 1-6 provide the sequences of the VHCDR1, VHCDR2, VHCDR3, VLCDR1, VLCDR2, VLCDR3 regions of the R-8 antibody anti-CD40 antibody respectively. SEQ ID Nos: 7 and 8 provide the sequences for the VH and VL regions of APX005M, the humanized version of the R-8 rabbit anti-CD40 antibody, without a signal peptide respectively. SEQ ID Nos: 9 and 10 provide the sequences of the VL and VH regions of the R-8 rabbit anti-CD40 antibody.

The methods described herein can use the concentrations and timings used in clinical trials for inhibitors, such as a checkpoint inhibitor or CD40 agonist, alone or in combination with a neoantigen vaccine or immunogenic composition. Topalian, et al. N Engl J Med 2012; 366:2443-2454 describes a phase 1 study that assessed the safety, antitumor activity, and pharmacokinetics of BMS-936558, a fully human IgG4-blocking monoclonal antibody directed against PD-1, in patients in need thereof with selected advanced solid tumors. The antibody was administered as an intravenous infusion every 2 weeks of each 8-week treatment cycle. Response was assessed after each treatment cycle. Patients received treatment for up to 2 years (12 cycles). Patients with advanced melanoma, non-small-cell lung cancer, renal-cell cancer, castration-resistant prostate cancer, or colorectal cancer were enrolled. Cohorts of three to six patients per dose level were enrolled sequentially at doses of 1.0, 3.0, or 10.0 mg per kilogram of body weight. Initially, five expansion cohorts of approximately 16 patients each were enrolled at doses of 10.0 mg per kilogram for melanoma, non-small-cell lung cancer, renal-cell cancer, castration-resistant prostate cancer, and colorectal cancer. On the basis of initial signals of activity, additional expansion cohorts of approximately 16 patients each were enrolled for melanoma (at a dose of 1.0 or 3.0 mg per kilogram, followed by cohorts randomly assigned to 0.1, 0.3, or 1.0 mg per kilogram), lung cancer (patients with the squamous or nonsquamous subtype, randomly assigned to a dose of 1.0, 3.0, or 10.0 mg per kilogram), and renal-cell cancer (at a dose of 1.0 mg per kilogram).

In some embodiments, a subject is administered APX005M at a dosage of 0.001 mg/kg to 3.5 mg/kg. In some embodiments, a subject is administered APX005M at a dosage of at least 0.001 mg/kg, 0.005 mg/kg, 0.01 mg/kg, 0.05 mg/kg, 0.1 mg/kg, 0.5 mg/kg, 1.0 mg/kg 1.5 mg/kg, 2.0 mg/kg, 2.5 mg/kg or 3 mg/kg. In some embodiments, a subject is administered APX005M at a dosage of at most 3.5 mg/kg, 3.0 mg/kg, 2.5 mg/kg, 2.0 mg/kg, 1.5 mg/kg, 1.0 mg/kg, 0.5 mg/kg, 0.1 mg/kg, 0.05 mg/kg or 0.01 mg/kg. In some embodiments, a subject is administered APX005M at a dosage of 0.001 mg/kg to 0.005 mg/kg, 0.001 mg/kg to 0.01 mg/kg, 0.001 mg/kg to 0.05 mg/kg, 0.001 mg/kg to 0.1 mg/kg, 0.001 mg/kg to 0.5 mg/kg, 0.001 mg/kg to 1 mg/kg, 0.001 mg/kg to 1.5 mg/kg, 0.001 mg/kg to 2 mg/kg, 0.001 mg/kg to 2.5 mg/kg, 0.001 mg/kg to 3 mg/kg, 0.001 mg/kg to 3.5 mg/kg, 0.005 mg/kg to 0.01 mg/kg, 0.005 mg/kg to 0.05 mg/kg, 0.005 mg/kg to 0.1 mg/kg, 0.005 mg/kg to 0.5 mg/kg, 0.005 mg/kg to 1 mg/kg, 0.005 mg/kg to 1.5 mg/kg, 0.005 mg/kg to 2 mg/kg, 0.005 mg/kg to 2.5 mg/kg, 0.005 mg/kg to 3 mg/kg, 0.005 mg/kg to 3.5 mg/kg, 0.01 mg/kg to 0.05 mg/kg, 0.01 mg/kg to 0.1 mg/kg, 0.01 mg/kg to 0.5 mg/kg, 0.01 mg/kg to 1 mg/kg, 0.01 mg/kg to 1.5 mg/kg, 0.01 mg/kg to 2 mg/kg, 0.01 mg/kg to 2.5 mg/kg, 0.01 mg/kg to 3 mg/kg, 0.01 mg/kg to 3.5 mg/kg, 0.05 mg/kg to 0.1 mg/kg, 0.05 mg/kg to 0.5 mg/kg, 0.05 mg/kg to 1 mg/kg, 0.05 mg/kg to 1.5 mg/kg, 0.05 mg/kg to 2 mg/kg, 0.05 mg/kg to 2.5 mg/kg, 0.05 mg/kg to 3 mg/kg, 0.05 mg/kg to 3.5 mg/kg, 0.1 mg/kg to 0.5 mg/kg, 0.1 mg/kg to 1 mg/kg, 0.1 mg/kg to 1.5 mg/kg, 0.1 mg/kg to 2 mg/kg, 0.1 mg/kg to 2.5 mg/kg, 0.1 mg/kg to 3 mg/kg, 0.1 mg/kg to 3.5 mg/kg, 0.5 mg/kg to 1 mg/kg, 0.5 mg/kg to 1.5 mg/kg, 0.5 mg/kg to 2 mg/kg, 0.5 mg/kg to 2.5 mg/kg, 0.5 mg/kg to 3 mg/kg, 0.5 mg/kg to 3.5 mg/kg, 1 mg/kg to 1.5 mg/kg, 1 mg/kg to 2 mg/kg, 1 mg/kg to 2.5 mg/kg, 1 mg/kg to 3 mg/kg, 1 mg/kg to 3.5 mg/kg, 1.5 mg/kg to 2 mg/kg, 1.5 mg/kg to 2.5 mg/kg, 1.5 mg/kg to 3 mg/kg, 1.5 mg/kg to 3.5 mg/kg, 2 mg/kg to 2.5 mg/kg, 2 mg/kg to 3 mg/kg, 2 mg/kg to 3.5 mg/kg, 2.5 mg/kg to 3 mg/kg, 2.5 mg/kg to 3.5 mg/kg, or 3 mg/kg to 3.5 mg/kg. In some embodiments, a subject is administered APX005M at a dosage of 0.001 mg/kg, 0.005 mg/kg, 0.01 mg/kg, 0.05 mg/kg, 0.1 mg/kg, 0.5 mg/kg, 1 mg/kg, 1.5 mg/kg, 2 mg/kg, 2.5 mg/kg, 3 mg/kg, or 3.5 mg/kg.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

The invention provides for pharmaceutical compositions containing at least one tumor specific neoantigen described herein. In embodiments, the pharmaceutical compositions contain a pharmaceutically acceptable carrier, excipient, or diluent, which includes any pharmaceutical agent that does not itself induce the production of an immune response harmful to a subject receiving the composition, and which may be administered without undue toxicity. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful for treating and/or preventing viral infection and/or autoimmune disease.

A thorough discussion of pharmaceutically acceptable carriers, diluents, and other excipients is presented in Remington's Pharmaceutical Sciences (17th ed., Mack Publishing Company) and Remington: The Science and Practice of Pharmacy (21st ed., Lippincott Williams & Wilkins), which are hereby incorporated by reference. The formulation of the pharmaceutical composition should suit the mode of administration. In embodiments, the pharmaceutical composition is suitable for administration to humans, and can be sterile, non-particulate and/or non-pyrogenic.

Pharmaceutically acceptable carriers, excipients, or diluents include, but are not limited, to saline, buffered saline, dextrose, water, glycerol, ethanol, sterile isotonic aqueous buffer, and combinations thereof.

Wetting agents, emulsifiers and lubricants, such as sodium lauryl sulfate and magnesium stearate, as well as coloring agents, release agents, coating agents, sweetening, flavoring and perfuming agents, preservatives, and antioxidants can also be present in the compositions.

Examples of pharmaceutically-acceptable antioxidants include, but are not limited to: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol and the like; and (3) metal chelating agents, such as citric acid, ethylenediamine tetraacetic acid (EDTA), sorbitol, tartaric acid, phosphoric acid, and the like.

In embodiments, the pharmaceutical composition is provided in a solid form, such as a lyophilized powder suitable for reconstitution, a liquid solution, suspension, emulsion, tablet, pill, capsule, sustained release formulation, or powder.

In embodiments, the pharmaceutical composition is supplied in liquid form, for example, in a sealed container indicating the quantity and concentration of the active ingredient in the pharmaceutical composition. In related embodiments, the liquid form of the pharmaceutical composition is supplied in a hermetically sealed container.

Methods for formulating the pharmaceutical compositions of the present invention are conventional and well known in the art (see Remington and Remington's). One of skill in the art can readily formulate a pharmaceutical composition having the desired characteristics (e.g., route of administration, biosafety, and release profile).

Methods for preparing the pharmaceutical compositions include the step of bringing into association the active ingredient with a pharmaceutically acceptable carrier and, optionally, one or more accessory ingredients. The pharmaceutical compositions can be prepared by uniformly and intimately bringing into association the active ingredient with liquid carriers, or finely divided solid carriers, or both, and then, if necessary, shaping the product. Additional methodology for preparing the pharmaceutical compositions, including the preparation of multilayer dosage forms, are described in Ansel's Pharmaceutical Dosage Forms and Drug Delivery Systems (9th cd., Lippincott Williams & Wilkins), which is hereby incorporated by reference.

Pharmaceutical compositions suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound(s) described herein, a derivative thereof, or a pharmaceutically acceptable salt or prodrug thereof as the active ingredient(s). The active ingredient can also be administered as a bolus, electuary, or paste.

In solid dosage forms for oral administration (e.g., capsules, tablets, pills, dragees, powders, granules and the like), the active ingredient is mixed with one or more pharmaceutically acceptable carriers, excipients, or diluents, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents, in the case of capsules, tablets, and pills, the pharmaceutical compositions can also comprise buffering agents. Solid compositions of a similar type can also be prepared using fillers in soft and hard-filled gelatin capsules, and excipients such as lactose or milk sugars, as well as high molecular weight polyethylene glycols and the like.

A tablet, can be made by compression or molding, optionally with one or more accessory ingredients. Compressed tablets can be prepared using binders (for example, gelatin or hydroxypropylmethyl cellulose), lubricants, inert diluents, preservatives, disintegrants (for example, sodium starch glycolate or cross-linked sodium carboxymethyl cellulose), surface-actives, and/or dispersing agents. Molded tablets can be made by molding in a suitable machine a mixture of the powdered active ingredient moistened with an inert liquid diluent.

The tablets and other solid dosage forms, such as dragees, capsules, pills, and granules, can optionally be scored or prepared with coatings and shells, such as enteric coatings and other coatings well known in the art.

In some embodiments, in order to prolong the effect of an active ingredient, it is desirable to slow the absorption of the compound from subcutaneous or intramuscular injection. This can be accomplished by the use of a liquid suspension of crystalline or amorphous material having poor water solubility. The rate of absorption of the active ingredient then depends upon its rate of dissolution which, in turn, can depend upon crystal size and crystalline form. Alternatively, delayed absorption of a parenterally-administered active ingredient is accomplished by dissolving or suspending the compound in an oil vehicle. In addition, prolonged absorption of the injectable pharmaceutical form can be brought about by the inclusion of agents that delay absorption such as aluminum monostearate and gelatin.

Controlled release parenteral compositions can be in form of aqueous suspensions, microspheres, microcapsules, magnetic microspheres, oil solutions, oil suspensions, emulsions, or the active ingredient can be incorporated in biocompatible carrier(s), liposomes, nanoparticles, implants or infusion devices.

Materials for use in the preparation of microspheres and/or microcapsules include biodegradable/bioerodible polymers such as polyglactin, poly-(isobutyl cyanoacrylate), poly(2-hydroxyethyl-L-glutamine) and poly(lactic acid).

Biocompatible carriers which can be used when formulating a controlled release parenteral formulation include carbohydrates such as dextrans, proteins such as albumin, lipoproteins or antibodies.

Materials for use in implants can be non-biodegradable, e.g., polydimethylsiloxane, or biodegradable such as, e.g., poly(caprolactone), poly(lactic acid), poly(glycolic acid) or poly(orthoesters).

In embodiments, the active ingredients) are administered by aerosol. This is accomplished by preparing an aqueous aerosol, liposomal preparation, or solid particles containing the compound. A non-aqueous (e.g., fluorocarbon propellant) suspension can be used. The pharmaceutical composition can also be administered using a sonic nebulizer, which would minimize exposing the agent to shear, which can result in degradation of the compound.

Ordinarily, an aqueous aerosol is made by formulating an aqueous solution or suspension of the active ingredient(s) together with conventional pharmaceutically-acceptable carriers and stabilizers. The carriers and stabilizers vary with the requirements of the particular compound, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Dosage forms for topical or transdermal administration of an active ingredient(s) includes powders, sprays, ointments, pastes, creams, lotions, gels, solutions, patches and inhalants. The active ingredient(s) can be mixed under sterile conditions with a pharmaceutically acceptable carrier, and with any preservatives, buffers, or propellants as appropriate.

Transdermal patches suitable for use in the present invention are disclosed in Transdermal Drug Delivery: Developmental Issues and Research Initiatives (Marcel Dekker Inc., 1989) and U.S. Pat. Nos. 4,743,249, 4,906,169, 5,198,223, 4,816,540, 5,422, 119, 5,023,084, which are hereby incorporated by reference. The transdermal patch can also be any transdermal patch well known in the art, including transscrotal patches. Pharmaceutical compositions in such transdermal patches can contain one or more absorption enhancers or skin permeation enhancers well known in the art (see, e.g., U.S. Pat. Nos. 4,379,454 and 4,973,468, which are hereby incorporated by reference). Transdermal therapeutic systems for use in the present invention can be based on iontophoresis, diffusion, or a combination of these two effects.

Transdermal patches have the added advantage of providing controlled delivery of active ingredient(s) to the body. Such dosage forms can be made by dissolving or dispersing the active ingredient(s) in a proper medium. Absorption enhancers can also be used to increase the flux of the active ingredient across the skin. The rate of such flu can be controlled by either providing a rate controlling membrane or dispersing the active ingredient(s) in a polymer matrix or gel.

Such pharmaceutical compositions can be in the form of creams, ointments, lotions, liniments, gels, hydrogels, solutions, suspensions, sticks, sprays, pastes, plasters and other kinds of transdermal drug delivery systems. The compositions can also include pharmaceutically acceptable carriers or excipients such as emulsifying agents, antioxidants, buffering agents, preservatives, humectants, penetration enhancers, chelating agents, gel-forming agents, ointment bases, perfumes, and skin protective agents.

Examples of emulsifying agents include, but are not limited to, naturally occurring gums, e.g. gum acacia or gum tragacanth, naturally occurring phosphatides, e.g. soybean lecithin and sorbitan monooleate derivatives.

Examples of antioxidants include, but are not limited to, butylated hydroxy anisole (BHA), ascorbic acid and derivatives thereof tocopherol and derivatives thereof, and cysteine.

Examples of preservatives include, but are not limited to, trehalose, parabens, such as methyl or propyl p-hydroxybenzoate and benzalkonium chloride.

Examples of humectants include, but are not limited to, glycerin, propylene glycol, sorbitol and urea.

Examples of penetration enhancers include, but are not limited to, propylene glycol, DMSO, triethanolamine, N,N-dimethylacetamide, N,N-dimethylforamamide, 2-pyrrolidone and derivatives thereof, tetrahydrofurfuryl alcohol, propylene glycol, diethylene glycol monoethyl or monomethyl ether with propylene glycol monolaurate or methyl laurate, eucalyptol, lecithin, TRANSCUTOL, and AZONE.

Examples of chelating agents include, but are not limited to, sodium EDTA, citric acid and phosphoric acid.

Examples of gel forming agents include, but are not limited to, Carbopol, cellulose derivatives, bentonite, alginates, gelatin and polyvinylpyrrolidone.

In addition to the active ingredient(s), the ointments, pastes, creams, and gels of the present invention can contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain excipients such as lactose, talc, silicic acid, aluminum hydroxide, calcium silicates and polyamide powder, or mixtures of these substances. Sprays can additionally contain customary propellants, such as chlorofluorohydrocarbons, and volatile unsubstituted hydrocarbons, such as butane and propane.

Injectable depot forms are made by forming microencapsule matrices of compound(s) of the invention in biodegradable polymers such as polylactide-polyglycolide. Depending on the ratio of compound to polymer, and the nature of the particular polymer employed, the rate of compound release can be controlled. Examples of other biodegradable polymers include poly(orthoesters) and poly(anhydrides). Depot injectable formulations are also prepared by entrapping the drug in liposomes or microemulsions which are compatible with body tissue.

Subcutaneous implants are well known in the art and are suitable for use in the present invention. Subcutaneous implantation methods are preferably non-irritating and mechanically resilient. The implants can be of matrix type, of reservoir type, or hybrids thereof. In matrix type devices, the carrier material can be porous or non-porous, solid or semi-solid, and permeable or impermeable to the active compound or compounds. The carrier material can be biodegradable or may slowly erode after administration. In some instances, the matrix is non-degradable but instead relies on the diffusion of the active compound through the matrix for the carrier material to degrade. Alternative subcutaneous implant methods utilize reservoir devices where the active compound or compounds are surrounded by a rate controlling membrane, e.g., a membrane independent of component concentration (possessing zero-order kinetics). Devices consisting of a matrix surrounded by a rate controlling membrane also suitable for use.

Both reservoir and matrix type devices can contain materials such as polydimethylsiloxane, such as SILASTIC, or other silicone rubbers. Matrix materials can be insoluble polypropylene, polyethylene, polyvinyl chloride, ethylvinyl acetate, polystyrene and polymethacrylate, as well as glycerol esters of the glycerol palmitostearate, glycerol stearate, and glycerol behenate type. Materials can be hydrophobic or hydrophilic polymers and optionally contain solubilizing agents.

Subcutaneous implant devices can be slow-release capsules made with any suitable polymer, e.g., as described in U.S. Pat. Nos. 5,035,891 and 4,210,644, which are hereby incorporated by reference.

In general, at least four different approaches are applicable in order to provide rate control over the release and transdermal permeation of a drug compound. These approaches are: membrane-moderated systems, adhesive diffusion-controlled systems, matrix dispersion-type systems and microreservoir systems. It is appreciated that a controlled release percutaneous and/or topical composition can be obtained by using a suitable mixture of these approaches.

In a membrane-moderated system, the active ingredient is present in a reservoir which is totally encapsulated in a shallow compartment molded from a drug-impermeable laminate, such as a metallic plastic laminate, and a rate-controlling polymeric membrane such as a microporous or a non-porous polymeric membrane, e.g., ethylene-vinyl acetate copolymer. The active ingredient is released through the rate controlling polymeric membrane. In the drug reservoir, the active ingredient can either be dispersed in a solid polymer matrix or suspended in an unleachable, viscous liquid medium such as silicone fluid. On the external surface of the polymeric membrane, a thin layer of an adhesive polymer is applied to achieve an intimate contact of the transdermal system with the skin surface. The adhesive polymer can be a polymer which is hypoallergenic and compatible with the active drug substance.

In an adhesive diffusion-controlled system, a reservoir of the active ingredient is formed by directly dispersing the active ingredient in an adhesive polymer and then by, e.g., solvent casting, spreading the adhesive containing the active ingredient onto a flat sheet of substantially drug-impermeable metallic plastic backing to form a thin drug reservoir layer.

A matrix dispersion-type system is characterized in that a reservoir of the active ingredient is formed by substantially homogeneously dispersing the active ingredient in a hydrophilic or lipophilic polymer matrix. The drag-containing polymer is then molded into disc with a substantially well-defined surface area and controlled thickness. The adhesive polymer is spread along the circumference to form a strip of adhesive around the disc.

A microreservoir system can be considered as a combination of the reservoir and matrix dispersion type systems. In this case, the reservoir of the active substance is formed by first suspending the drug solids in an aqueous solution of water-soluble polymer and then dispersing the drug suspension in a lipophilic polymer to form a multiplicity of unleachable, microscopic spheres of drug reservoirs.

Any of the herein-described controlled release, extended release, and sustained release compositions can be formulated to release the active ingredient in about 30 minutes to about I week, in about 30 minutes to about 72 hours, in about 30 minutes to 24 hours, in about 30 minutes to 12 hours, in about 30 minutes to 6 hours, in about 30 minutes to 4 hours, and in about 3 hours to 10 hours. In embodiments, an effective concentration of the active ingredient(s) is sustained in a subject for 4 hours, 6 hours, 8 hours, 10 hours, 12 hours, 16 hours, 24 hours, 48 hours, 72 hours, or more after administration of the pharmaceutical compositions to the subject.

### VII. Vaccine or Immunogenic Compositions

The present invention is directed to methods of combination treatment. The combination treatment comprises at least an immunogenic composition, e.g., a neoplasia vaccine or immunogenic composition capable of raising a specific T-cell response. The neoplasia vaccine or immunogenic composition comprises neoantigenic peptides and/or neoantigenic polypeptides corresponding to tumor specific neoantigens identified by the methods described herein. The combination treatment also comprises at least one inhibitor, such as a checkpoint inhibitor or CD40 agonist. In particular, the present invention is directed to methods of treating or preventing a neoplasia comprising the steps of administering to a subject (a) a neoplasia vaccine or immunogenic composition, and (h) at least one inhibitor, such as a checkpoint inhibitor or CD40 agonist.

A suitable neoplasia vaccine or immunogenic composition can contain a plurality of tumor specific neoantigenic peptides. In an embodiment, the vaccine or immunogenic composition can include between 1 and 100 sets of peptides, between 1 and 50 such peptides, between 10 and 30 sets peptides, or between 15 and 25 peptides. According to another embodiment, the vaccine or immunogenic composition can include at least one peptides, such as 2, 3, 4, or 5 peptides, In certain embodiments, the vaccine or immunogenic composition can comprise 5, 6, 7, 8, 9, 10, 11, 12, 13, 14, 15, 16, 17, 18, 19, 20, 21, 22, 23, 24, 25, 26, 27, 28, 29, or 30 different peptides. Multiple doses of the vaccine or immunogenic composition may be administered to a subject. Each dose of the vaccine composition may comprise different sets of peptides. For instance, one dose or part of one dose of the composition may comprise 5 peptides. Another part of the dose may comprise a different set of 5 peptides.

The optimum amount of each peptide to be included in the vaccine or immunogenic composition and the optimum dosing regimen can be determined by one skilled in the art without undue experimentation. For example, the peptide or its variant may be prepared for intravenous (i.v.) injection, sub-cutaneous (s.c.) injection, intradermal (i.d.) injection, intraperitoneal (i.p.) injection, intramuscular (i.m.) injection. Methods of peptide injection include s.c, i.d., i.p., i.m., and i.v. Methods of DNA injection include i.d., i.m., s.c, i.p. and i.v. For example, doses of between 1 and 500 mg, 50 μg and 1.5 mg, or 10 μg to 500 μg, of peptide or DNA may be given and can depend from the respective peptide or DNA. Doses of this range were successfully used in previous trials (Brunsvig P F, et al., Cancer Immunol Immunother. 2006; 55(12): 1553-1564; M. Staehler, et al, ASCO meeting 2007; Abstract No 3017). Other methods of administration of the vaccine or immunogenic composition are known to those skilled in the art.

The vaccine or immunogenic composition may be administered to a subject in the form of one or more subcutaneous injections. In one embodiment, a single dose of the composition may be divided into one or more subcutaneous injections for administration. For instance, a single dose of the composition may be divided in to 1, 2, 3, 4, 5, 6, 7 or 8 different subcutaneous injections. Each injection of the composition may comprise one or more peptides. The peptides in each injection of a single dose of the composition may comprise different sets of peptides. In some examples, each injection of a single dose of the composition comprises 1, 2, 3, 4, 5 or 6 different peptides. Alternatively, each subcutaneous injection of a single dose of the composition may comprise the same set of peptides. In some cases, multiple injections, as part of a single dose of the vaccine or the immunogenic composition may comprise different sets of peptides. For instance, a single dose of the composition may be divided in to 4 injections with each injection comprising 5 different sets of peptides.

The vaccine or immunogenic composition may be administered to the patient as a subcutaneous injection in a single location. For example, a single dose of the vaccine or immunogenic composition may be administered to the patient as one injection in an extremity. In cases where a single dose of the composition is divided over one or more injections, the dose may be administered into a subject in multiple locations. For instance, a case where a dose is divided in to 4 different injections, the 4 different injections may be administered into different extremities. In some cases, multiple injections as part of a single dose of the composition may be administered at the same location at different time periods. For instance, a 5, 10, 15, 20, 30, 50 or 60 minute time period may be provided between different injections of one single dose of the vaccine or immunogenic composition.

In one embodiment of the present invention the different tumor specific neoantigenic peptides and/or polypeptides are selected for use in the neoplasia vaccine or immunogenic composition so as to maximize the likelihood of generating an immune attack against the neoplasia/tumor of the patient. Without being bound by theory, it is believed that the inclusion of a diversity of tumor specific neoantigenic peptides can generate a broad scale immune attack against a neoplasia/tumor. In one embodiment, the selected tumor specific neoantigenic peptides/polypeptides are encoded by missense mutations. In a second embodiment, the selected tumor specific neoantigenic peptides polypeptides are encoded by a combination of missense mutations and neoORF mutations. In a third embodiment, the selected tumor specific neoantigenic peptides/polypeptides are encoded by neoORF mutations.

In one embodiment in which the selected tumor specific neoantigenic peptides/polypeptides are encoded by missense mutations, the peptides and/or polypeptides are chosen based on their capability to associate with the particular MHC molecules of the patient. Peptides/polypeptides derived from neoORF mutations can also be selected on the basis of their capability to associate with the particular MHC molecules of the patient, but can also be selected even if not predicted to associate with the particular MHC molecules of the patient.

The vaccine or immunogenic composition is capable of raising a specific cytotoxic T cells response and/or a specific helper T-cell response.

The vaccine or immunogenic composition can further comprise an adjuvant and/or a carrier. Examples of useful adjuvants and carriers are given herein. The peptides and/or polypeptides in the composition can be associated with a carrier such as, e.g., a protein or an antigen-presenting cell such as e.g. a dendritic cell (DC) capable of presenting the peptide to a T-cell.

Adjuvants are any substance whose admixture into the vaccine or immunogenic composition increases or otherwise modifies the immune response to the mutant peptide. Carriers are scaffold structures, for example a polypeptide or a polysaccharide, to which the neoantigenic peptides, is capable of being associated. Optionally, adjuvants are conjugated covalently or non-covalently to the peptides or polypeptides of the invention.

The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th2 response into a primarily cellular, or Th1 response.

Suitable adjuvants include, but are not limited to 1018 ISS, aluminum salts, Amplivax, AS 15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, 0M-197-NIP-EC, ONTAK, PEPTEL vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox, Quil or Superfos. Several immunological adjuvants (e.g., F59) specific for dendritic cells and their preparation have been described previously (Dupuis M, et al, Cell Immunol. 1998; 186(1): 18-27; Allison A C; Dev Biol Stand. 1998; 92:3-11). Also cytokines may be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al, J Immunother Emphasis Tumor Immunol. 1996 (6):414-418).

Toll like receptors (TLRs) may also be used as adjuvants, and are important members of the family of pattern recognition receptors (PRRs) which recognize conserved motifs shared by many micro-organisms, termed “pathogen-associated molecular patterns” (PAMPS). Recognition of these “danger signals” activates multiple elements of the innate and adaptive immune system. ‘TLRs are expressed by cells of the innate and adaptive immune systems such as dendritic cells (DCs), macrophages, T and B cells, mast cells, and granulocytes and are localized in different cellular compartments, such as the plasma membrane, lysosomes, endosomes, and endo lysosomes. Different TLRs recognize distinct PAMPS. For example, TLR4 is activated by LPS contained in bacterial cell walls, TLR 9 is activated by unmethylated bacterial or viral CpG DNA, and TLRS is activated by double stranded RNA. TLR ligand binding leads to the activation of one or more intracellular signaling pathways, ultimately resulting in the production of many key molecules associated with inflammation and immunity (particularly the transcription factor NF-κB and the Type-I interferons). TLR mediated DC activation leads to enhanced DC activation, phagocytosis, upregulation of activation and co-stimulation markers such as CD80, CD83, and CD86, expression of CCR7 allowing migration of DC to draining lymph nodes and facilitating antigen presentation to T cells, as well as increased secretion of cytokines such as type I interferons, IL-12, and IL-6. All of these downstream events are critical for the induction of an adaptive immune response.

Among the most promising cancer vaccine or immunogenic composition adjuvants currently in clinical development are the TLR9 agonist CpG and the synthetic double-stranded RNA (dsRNA) TLR3 ligand poly-ICLC. In preclinical studies poly-ICLC appears to be the most potent TLR adjuvant when compared to LPS and CpG due to its induction of pro-inflammatory cytokines and lack of stimulation of IL-IO, as well as maintenance of high levels of co-stimulatory molecules in IX s i. Furthermore, poly-ICLC was recently directly compared to CpG in non-human primates (rhesus macaques) as adjuvant for a protein vaccine or immunogenic composition consisting of human papillomavirus (HPV)16 capsomers (Stahl-Hennig C, Eisenblatter M, Jasny E, et al. Synthetic double-stranded R As are adjuvants for the induction of T helper I and humoral immune responses to human papillomavirus in rhesus macaques. PLoS pathogens. April 2009; 5(4)).

CpG immuno stimulatory oligonucleotides have also been reported to enhance the effects of adjuvants in a vaccine or immunogenic composition setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly, it enhances dendritic cell maturation and differentiation, resulting in enhanced activation of Th1 cells and strong cytotoxic T-lymphocyte (CTL) generation, even in the absence of CD4 T-cell help. The Th1 bias induced by TLR9 stimulation is maintained even in the presence of vaccine adjuvants such as alum or incomplete Freund's adjuvant (IFA) that normally promote a Th2 bias. CpG oligonucleotides show even greater adjuvant activity when formulated or co-administered with other adjuvants or in formulations such as microparticles, nanoparticles, lipid, emulsions or similar formulations, which are especially necessary for inducing a strong response when the antigen is relatively weak. They also accelerate the immune response and enabled the antigen doses to be reduced by approximately two orders of magnitude, with comparable antibody responses to the full-dose vaccine without CpG in some experiments (Arthur M. Krieg, Nature Reviews, Drug Discovery, 5, Jun. 2006, 471-484). U.S. Pat. No. 6,406,705 describes the combined use of CpG oligonucleotides, non-nucleic acid adjuvants and an antigen to induce an antigen-specific immune response. A commercially available CpG TLR9 antagonist is dSLIM (double Stem Loop Immunomodulator) by Mologen (Berlin, GERMANY), which can be a component of the pharmaceutical composition of the present invention. Other TLR binding molecules such as RNA binding TLR 7, TLR 8 and/or TLR 9 may also be used.

Other examples of useful adjuvants include, but. are not limited to, chemically modified CpGs (e.g. CpR, Idera), Poly(I:C)(e.g. polyI:CI2U), non-CpG bacterial DNA or RNA as well as immunoactive small molecules and antibodies such as cyclophosphamide, sunitinib, bevacizumab, Celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives useful in the context of the present invention can readily be determined by the skilled artisan without undue experimentation. Additional adjuvants include colony-stimulating factors, such as Granulocyte Macrophage Colony Stimulating Factor (GM-CSF, sargramostim).

Poly-ICLC is a synthetically prepared double-stranded RNA consisting of polyI and polyC strands of average length of about 5000 nucleotides, which has been stabilized to thermal denaturation and hydrolysis by serum nucleases by the addition of polylysine and carboxymethylcellulose. The compound activates TLR3 and the RNA helicase-domain of MDA5, both members of the PAMP family, leading to DC and natural killer (NK) cell activation and production of a “natural mix” of type I interferons, cytokines, and chemokines. Furthermore, poly-ICLC exerts a more direct, broad host-targeted anti-infectious and possibly antitumor effect mediated by the two IFN-inducible nuclear enzyme systems, the 2′5′-OAS and the P1/eIF2a kinase, also known as the PKR (4-6), as well as RIG-I helicase and MDA5.

In rodents and non-human primates, poly-ICLC was shown to enhance T cell responses to viral antigens, cross-priming, and the induction of tumor-, virus-, and autoantigen-specific CD8+ T-cells. In a recent study in non-human primates, poly-ICLC was found to be essential for the generation of antibody responses and T-cell immunity to DC targeted or non-targeted HIV Gag p24 protein, emphasizing its effectiveness as a vaccine adjuvant.

In human subjects, transcriptional analysis of serial whole blood samples revealed similar gene expression profiles among the 8 healthy human volunteers receiving one single s.c. administration of poly-ICLC and differential expression of up to 212 genes between these 8 subjects versus 4 subjects receiving placebo. Remarkably, comparison of the poly-ICLC gene expression data to previous data from volunteers immunized with the highly effective yellow fever vaccine YF17D showed that a large number of transcriptional and signal transduction canonical pathways, including those of the innate immune system, were similarly upregulated at peak time points.

More recently, an immunologic analysis was reported on patients with ovarian, fallopian tube, and primary peritoneal cancer in second or third complete clinical remission who were treated on a phase 1 study of subcutaneous vaccination with synthetic overlapping long peptides (OLP) from the cancer testis antigen NY-ESO-1 alone or with Montanide-ISA-51, or with 1.4 mg poly-ICLC and Montanide. The generation of NY-ESO-1-specific CD4+ and CD 8+ T-cell and antibody responses were markedly enhanced with the addition of poly-ICLC and Montanide compared to OLP alone or OLP and Montanide.

A vaccine or immunogenic composition according to the present invention may comprise more than one different adjuvant. Furthermore, the invention encompasses a therapeutic composition comprising any adjuvant substance including any of those herein discussed. It is also contemplated that the peptide or polypeptide, and the adjuvant can be administered separately in any appropriate sequence.

A carrier may be present independently of an adjuvant. The carrier may be covalently linked to the antigen. A carrier can also be added to the antigen by inserting DNA encoding the carrier in frame with DNA encoding the antigen. The function of a carrier can for example be to confer stability, to increase the biological activity, or to increase serum half-life. Extension of the half-life can help to reduce the number of applications and to lower doses, thus are beneficial for therapeutic but also economic reasons. Furthermore, a carrier may aid presenting peptides to T-cells. The carrier may be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier may be a physiologically acceptable carrier acceptable to humans and safe. However, tetanus toxoid and/or diphtheria toxoid are suitable carriers in one embodiment of the invention. Alternatively, the carrier may be dextrans for example sepharose.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is only possible if a trimeric complex of peptide antigen, MHC molecule, and APC is present. Correspondingly, it may enhance the immune response if not only the peptide is used for activation of CTLs, but if additionally APCs with the respective MHC molecule are added. Therefore, in some embodiments the vaccine or immunogenic composition according to the present invention additionally contains at least one antigen presenting cell.

The antigen-presenting cell (or stimulator cell) typically has an MHC class I or II molecule on its surface, and in one embodiment is substantially incapable of itself loading the MHC class I or II molecule with the selected antigen. As is described in more detail herein, the MHC class I or II molecule may readily be loaded with the selected antigen in vitro.

CD8+ cell activity may be augmented through the use of CD4+ cells. The identification of CD4 T+ cell epitopes for tumor antigens has attracted interest because many immune based therapies against cancer may be more effective if both CD8+ and CD4+ T lymphocytes are used to target a patient's tumor, CD4+ cells are capable of enhancing CD8 T cell responses. Many studies in animal models have clearly demonstrated better results when both CD4+ and CD8+ T cells participate in anti-tumor responses (see e.g., Nishimura et al. (1999) Distinct role of antigen-specific T helper type I (Th1) and Th2 cells in tumor eradication in vivo. J Ex Med 190:617-27). Universal CD4+ T cell epitopes have been identified that are applicable to developing therapies against different types of cancer (see e.g., Kobayashi et al. (2008) Current Opinion in Immunology 20:221-27). For example, an HLA-DR restricted helper peptide from tetanus toxoid was used in melanoma vaccines to activate CD4+ T cells nonspecifically (see e.g., Slingluff et al. (2007) Immunologic and Clinical Outcomes of a Randomized Phase II Trial of Two Multipeptide Vaccines for Melanoma in the Adjuvant Setting, Clinical Cancer Research 13(21):6386-95). It is contemplated within the scope of the invention that such CD4+ cells may be applicable at three levels that vary in their tumor specificity: 1) a broad level in which universal CD4+ epitopes (e.g., tetanus toxoid) may be used to augment CD 8+ cells; 2) an intermediate level in which native, tumor-associated CD4+ epitopes may be used to augment CD8+ cells; and 3) a patient specific level in which neoantigen CD4+ epitopes may be used to augment CD8+ cells in a patient specific manner.

CD8+ cell immunity may also be generated with neoantigen loaded dendritic cell (DC) vaccine. DCs are potent antigen-presenting cells that initiate T cell immunity and can be used as cancer vaccines when loaded with one or more peptides of interest, for example, by direct peptide injection. For example, patients that were newly diagnosed with metastatic melanoma were shown to be immunized against 3 HLA-A*0201-restricted gp100 melanoma antigen-derived peptides with autologous peptide pulsed CD40 L/IFN-g-activated mature DCs via an IL-12p70-producing patient DC vaccine (see e.g., Carreno et al (2013) L-12p70-producing patient DC vaccine elicits Tel-polarized immunity, Journal of Clinical Investigation, 123(8):3383-94 and Ali et al. (2009) In situ regulation of DC subsets and T cells mediates tumor regression in mice, Cancer Immunotherapy, 1(8): 1-10). It is contemplated within the scope of the invention that neoantigen loaded DCs may be prepared using the synthetic TLR 3 agonist Polyinosinic-Polycytidylic Acid-poly-L-lysine Carboxymethylcellulose (Poly-ICLC) to stimulate the DCs. Poly-ICLC is a potent individual maturation stimulus for human DCs as assessed by an upregulation of CD83 and CD86, induction of interleukin-12 (IL-I2), tumor necrosis factor (TNF), interferon gamma-induced protein 10 (IP-10), interleukin 1 (IL-1), and type I interferons (IFN), and minimal interleukin 10 (IL-10) production. DCs may be differentiated from frozen peripheral blood mononuclear cells (PBMCs) obtained by leukopheresis, while PBMCs may be isolated by Ficoll gradient centrifugation and frozen in aliquots.

Illustratively, the following 7 day activation protocol may be used. Day 1—PBMCs are thawed and plated onto tissue culture flasks to select for monocytes which adhere to the plastic surface after 1-2 hr incubation at 37° C. in the tissue culture incubator. After incubation, the lymphocytes are washed off and the adherent monocytes are cultured for 5 days in the presence of interleukin-4 (IL-4) and granulocyte macrophage-colony stimulating factor (GM-CSF) to differentiate to immature DCs. On Day 6, immature DCs are pulsed with the keyhole limpet hemocyanin (KLH) protein which serves as a control for the quality of the vaccine and may boost the immunogenicity of the vaccine. The DCs are stimulated to mature, loaded with peptide antigens, and incubated overnight. On Day 7, the cells are washed, and frozen in 1 ml aliquots containing 4-20×10(6) cells using a controlled-rate freezer. Lot release testing for the batches of DCs may be performed to meet minimum specifications before the DCs are injected into patients (see e.g., Sabado et al. (2013) Preparation of tumor antigen-loaded mature dendritic cells for immunotherapy, J. Vis Exp. August 1; (78). doi: 10.3791/50085).

A DC vaccine may be incorporated into a scaffold system to facilitate delivery to a patient. Therapeutic treatment of a patients neoplasia with a DC vaccine may utilize a biomaterial system that releases factors that recruit host dendritic cells into the device, differentiates the resident, immature DCs by locally presenting adjuvants (e.g., danger signals) while releasing antigen, and promotes the release of activated, antigen loaded DCs to the lymph nodes (or desired site of action) where the DCs may interact, with T cells to generate a potent cytotoxic T lymphocyte response to the cancer neoantigens. Implantable biomaterials may be used to generate a potent cytotoxic T lymphocyte response against a neoplasia in a patient specific manner. The biomaterial-resident dendritic cells may then be activated by exposing them to danger signals mimicking infection, in concert with release of antigen from the biomaterial. The activated dendritic cells then migrate from the biomaterials to lymph nodes to induce a cytotoxic T effector response. This approach has previously been demonstrated to lead to regression of established melanoma in preclinical studies using a lysate prepared from tumor biopsies (see e.g., Ali et al. (2209) In situ regulation of DC subsets and T cells mediates tumor regression in mice, Cancer Immunotherapy 1 (8): 1-10; Ali et al. (2009)

In some embodiments, the antigen presenting cells are dendritic cells. Suitably, the dendritic cells are autologous dendritic cells that are pulsed with the neoantigenic peptide. The peptide may be any suitable peptide that gives rise to an appropriate T-cell response. T-cell therapy using autologous dendritic cells pulsed with peptides from a tumor associated antigen is disclosed in Murphy et al. (1996) The Prostate 29, 371-380 and Tjua et al. (1997) The Prostate 32, 272-278.

Thus, in one embodiment of the present invention the vaccine or immunogenic composition containing at least one antigen presenting cell is pulsed or loaded with one or more peptides of the present invention. Alternatively, peripheral blood mononuclear cells (PBMCs) isolated from a patient may be loaded with peptides in vivo and injected back into the patient. As an alternative the antigen presenting cell comprises an expression construct encoding a peptide of the present invention. The polynucleotide may be any suitable polynucleotide and is capable of transducing the dendritic cell, thus resulting in the presentation of a peptide and induction of immunity.

The inventive pharmaceutical composition may be compiled so that the selection, number and/or amount of peptides present in the composition is/are tissue, cancer, and/or patient-specific. For instance, the exact selection of peptides can be guided by expression patterns of the parent proteins in a given tissue to avoid side effects. The selection may be dependent on the specific type of cancer, the status of the disease, earlier treatment regimens, the immune status of the patient, and, of course, the HLA-haplotype of the patient. Furthermore, the vaccine or immunogenic composition according to the invention can contain individualized components, according to personal needs of the particular patient. Examples include varying the amounts of peptides according to the expression of the related neoantigen in the particular patient, unwanted side-effects due to personal allergies or other treatments, and adjustments for secondary treatments following a first round or scheme of treatment.

Pharmaceutical compositions comprising the peptide of the invention may be administered to an individual already suffering from cancer. In therapeutic applications, compositions are administered to a patient in an amount sufficient to elicit an effective CTL response to the tumor antigen and to cure or at least partially arrest symptoms and/or complications. An amount adequate to accomplish this is defined as “therapeutically effective dose.” Amounts effective for this use can depend on, e.g., the peptide composition, the manner of administration, the stage and severity of the disease being treated, the weight and general state of health of the patient, and the judgment of the prescribing physician, but generally range for the initial immunization (that is for therapeutic or prophylactic administration) from about 1.0 μg to about 50,000 μg of peptide for a 70 kg patient, followed by boosting dosages or from about 1.0 μg to about 10,000 μg of peptide pursuant to a boosting regimen over weeks to months depending upon the patient's response and condition and possibly by measuring specific CTL activity in the patient's blood. It should be kept in mind that the peptide and compositions of the present invention may generally be employed in serious disease states, that is, life-threatening or potentially life threatening situations, especially when the cancer has metastasized. For therapeutic use, administration should begin as soon as possible after the detection or surgical removal of tumors. This is followed by boosting doses until at least symptoms are substantially abated and for a period thereafter.

The pharmaceutical compositions (e.g., vaccine compositions) for therapeutic treatment are intended for parenteral, topical, nasal, oral or local administration. In some embodiments, the pharmaceutical compositions are administered parenterally, e.g., intravenously, subcutaneously, intradermally, or intramuscularly. The compositions may be administered at the site of surgical excision to induce a local immune response to the tumor. The invention provides compositions for parenteral administration which comprise a solution of the peptides and vaccine or immunogenic compositions are dissolved or suspended in an acceptable carrier, for example, an aqueous carrier. A variety of aqueous carriers may be used, e.g., water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

A liposome suspension containing a peptide may be administered intravenously, locally, topically, etc. in a dose which varies according to, inter alia, the manner of administration, the peptide being delivered, and the stage of the disease being treated. For targeting to the immune cells, a ligand, such as, e.g., antibodies or fragments thereof specific for cell surface determinants of the desired immune system cells, can be incorporated into the liposome.

For solid compositions, conventional or nanoparticle nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and for example, at a concentration of 25%-75%.

For aerosol administration, the immunogenic peptides can be supplied in finely divided form along with a surfactant and propellant. Typical percentages of peptides are 0.01%-20% by weight, for example, 1%-10%. The surfactant can, of course, be nontoxic, and soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, for example, 0.25-5%. The balance of the composition is ordinarily propellant. A carrier can also be included as desired, as with, e.g., lecithin for intranasal delivery.

The peptides and polypeptides of the invention can be readily synthesized chemically utilizing reagents that are free of contaminating bacterial or animal substances (Merrifield RB: Solid phase peptide synthesis. I. The synthesis of a tetrapeptide. J. Am. Chem, Soc. 85:2149-54, 1963).

The peptides and polypeptides of the invention can also be expressed by a vector, e.g., a nucleic acid molecule as herein-discussed, e.g., RNA or a DNA plasmid, a viral vector such as a poxvirus, e.g., orthopox virus, avipox virus, or adenovirus, AAV or lentivirus. This approach involves the use of a vector to express nucleotide sequences that encode the peptide of the invention. Upon introduction into an acutely or chronically infected host or into a non-infected host, the vector expresses the immunogenic peptide, and thereby elicits a host CTL response.

For therapeutic or immunization purposes, nucleic acids encoding the peptide of the invention and optionally one or more of the peptides described herein can also be administered to the patient. A number of methods are conveniently used to deliver the nucleic acids to the patient. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles. Generally, a plasmid for a vaccine or immunological composition can comprise DNA encoding an antigen (e.g., one or more neoantigens) operatively linked to regulatory sequences which control expression or expression and secretion of the antigen from a host cell, e.g., a mammalian cell; for instance, from upstream to downstream, DNA for a promoter, such as a mammalian virus promoter (e.g., a CMV promoter such as an hCMV or mCMV promoter, e.g., an early-intermediate promoter, or an SV40 promoter—see documents cited or incorporated herein for useful promoters), DNA for a eukaryotic leader peptide for secretion (e.g., tissue plasminogen activator), DNA for the neoantigen(s), and DNA encoding a terminator (e.g., the 3′ UTR transcriptional terminator from the gene encoding Bovine Growth Hormone or bGH polyA). A composition can contain more than one plasmid or vector, whereby each vector contains and expresses a different neoantigen. Mention is also made of Wasmoen U.S. Pat. No. 5,849,303, and Dale U.S. Pat. No. 5,811,104, whose text may be useful DNA or DNA plasmid formulations can be formulated with or inside cationic lipids; and, as to cationic lipids, as well as adjuvants, mention is also made of Loosmore U.S. Patent Application 2003/0104008. Also, teachings in Audonnet U.S. Pat. Nos. 6,228,846 and 6,159,477 may be relied upon for DNA plasmid teachings that can be employed in constructing and using DNA plasmids that contain and express i vivo.

The nucleic acids can also be delivered complexed to cationic compounds, such as cationic lipids. Lipid-mediated gene delivery methods are described, for instance, in WO 1996/18372; WO 1993/24640; Mannino & Gould-Fogerite, BioTechniques 6(7): 682-691 (1988); U.S. Pat. No. 5,279,833; WO 1991/06309; and Feigner et al., Proc. Natl. Acad. Sci. USA 84: 7413-7414 (1987).

RNA encoding the peptide of interest (e.g., mRNA) can also be used for delivery (see, e.g., Kiken et al, 2011; Su et al, 2011; see also U.S. Pat. No. 8,278,036; Halabi et al. J Clin Oncol (2003) 21: 1232-1237; Petsch et al, Nature Biotechnology 2012 December 7; 30(12): 1210-6).

Information concerning poxviruses that may be used in the practice of the invention, such as Chordopoxvirinae subfamily poxviruses (poxviruses of vertebrates), for instance, orthopoxviruses and avipoxviruses, e.g., vaccinia virus (e.g., Wyeth Strain, WR Strain (e.g., ATCC® VR-1354), Copenhagen Strain, NYVAC, NYVAC. 1, NYVAC.2, MVA, VA-BN), canarypox virus (e.g., Wheatley C93 Strain, ALVAC), fowlpox virus (e.g., FP9 Strain, Webster Strain, TROVAC), dovepox, pigeonpox, quail pox, and raccoon pox, inter alia, synthetic or non-naturally occurring recombinants thereof, uses thereof, and methods for making and using such recombinants may be found in scientific and patent literature, such as: U.S. Pat. Nos. 4,603,112, 4,769,330, 5,110,587, 5,174,993, 5,364,773, 5,762,938, 5,494,807, 5,766,597, 7,767,449, 6,780,407, 6,537,594, 6,265,189, 6,214,353, 6,130,066, 6,004,777, 5,990,091, 5,942,235, 5,833,975, 5,766,597, 5,756,101, 7,045,313, 6,780,417, 8,470,598, 8,372,622, 8,268,329, 8,268,325, 8,236,560, 8,163,293, 7,964,398, 7,964,396, 7,964,395, 7,939,086, 7,923,017, 7,897,156, 7,892,533, 7,628,980, 7,459,270, 7,445,924, 7,384,644, 7,335,364, 7,189,536, 7,097,842, 6,913,752, 6,761,893, 6,682,743, 5,770,212, 5,766,882, and 5,989,562, and Panicali, D. Proc. Natl. Acad. Sci. 1982; 79; 4927-493, Panicali D. Proc. Natl. Acad. Sci. 1983; 80(17): 5364-8, Mackett, M. Proc. Natl. Acad. Sci. 1982; 79: 7415-7419, Smith G L. Proc, Natl. Acad. Sci. 1983; 80(23): 7155-9, Smith G L. Nature 1983; 302: 490-5, Sullivan V J. Gen. Vir. 1987; 68: 2587-98, Perkus M Journal of Leukocyte Biology 1995; 58: 1-13, Yilma T D. Vaccine 1989; 7: 484-485, Brochier B. Nature 1991; 354: 520-22, Wiktor, T J. Proc. Natl Acad, Sci. 1984; 81:7194-8, Rupprecht, C E. Proc, Natl Acd. Sci. 1986; 83: 7947-50, Poulet, H Vaccine 2007; 25(Jul.): 5606-12, Weyer J. Vaccine 2009; 27(Nov.): 7198-201, Buller, R M Nature 1985; 317(6040): 813-5, Boiler R M. J. Virol. 1988; 62(3):866-74, Flexner, C. Nature 1987; 330(6145): 259-62, Shida, H. J. Virol. 1988; 62(12): 4474-80, Kotwal G J. J. Virol. 1989; 63(2): 600-6, Child, S J. Virology 1990; 174(2): 625-9, Mayr A. Zentralbl Bakteriol 1978; 167(5,6): 375-9, Antoine G. Virology. 1998; 244(2): 365-96, Wyatt, L S. Virology 1998; 251(2): 334-42, Sancho, M C. J. Virol. 2002; 76(16); 8313-34, Gallego-Gomez, J C. J. Virol. 2003; 77(19); 10606-22), Goebel S J. Virology 1990; (a,b) 179: 247-66, Tartaglia, J. Virol. 1992; 188(1): 217-32, Najera J L. J. Virol. 2006; 80(12): 6033-47, Najera, J L. J. Virol. 2006; 80: 6033-6047, Gomez, C E. J. Gen. Virol. 2007; 88: 2473-78, Mooij, P. Jour. Of Virol. 2008; 82: 2975-2988, Gomez, C E. Curr. Gene Ther. 2011; 11: 189-217, Cox, W. Virology 1993; 195: 845-50, Perkus, M. Jour. Of Leukocyte Biology 1995; 58: 1-13, Blanchard T J. J Gen Virology 1998; 79(5): 1159-67, Amara R. Science 2001; 292: 69-74, Hel, Z., J. Immunol. 2001; 167: 7180-9, Gherardi M M. J. Virol. 2003; 77: 7048-57, Didierlaurent, A. Vaccine 2004; 22: 3395-3403, Bissht H. Proc. Nat. Aca. Sci. 2004; 101: 6641-46, McCurdy L H. Clin. Inf. Dis 2004; 38: 1749-53, Earl P L. Nature 2004; 428: 182-85, Chen Z. J. Virol. 2005; 79: 2678-2688, Najera J L. J. Virol. 2006; 80(12): 6033-47, Nam J H. Acta. Virol. 2007; 51: 125-30, Antonis A F. Vaccine 2007; 25: 4818-4827, B Weyer J. Vaccine 2007; 25: 4213-22, Ferrier-Rembert A. Vaccine 2008; 26(14): 1794-804, Corbett M. Proc. Natl. Acad. Sci. 2008: 105(6): 2046-51, Kaufman H L., J. Clin. Oncol. 2004; 22: 2122-32, Amato, R J. Clin. Cancer Res. 2008; 14(22): 7504-10, Dreicer R. Invest New Drugs 2009; 27(4): 379-86, Kantoff P W. J. Clin. Oncol. 2010, 28, 1099-1105, Amato R J. J. Clin. Can. Res. 2010; 16(22): 5539-47, Kim, D W. Hum. Vaccine. 2010: 6: 784-791, Oudard, S. Cancer Immunol.]mm another. 2011; 60: 261-71, Wyatt, L S. Aids Res. Hum. Retroviruses. 2004; 20: 645-53, Gomez, C E. Virus Research 2004; 105: 11-22, Webster, D P. Proc. Natl Acad. Sci. 2005; 102; 4836-4, Huang, X. Vaccine 2007; 25: 8874-84, Gomez, C E. Vaccine 2007a; 25: 2863-85, Esteban M. Hum. Vaccine 2009; 5: 867-871, Gomez, C E. Curr. Gene therapy 2008; 8(2): 97-120, Whelan, K T. PLoS One 2009; 4(6): 5934, Scriba, T J. Eur, Jour. Immuno. 2010; 40(1): 279-90, Corbett, M. Proc. Natl, Acad. Sci. 2008; 105: 2046-2051, Midgley, C M. J. Gen. Virol. 2008; 89: 2992-97, Von Krempelhuber, A. Vaccine 2010; 28: 1209-16, Perreau, M. J. Of Virol. 2011; October: 9854-62, Pantaleo, G. Curr Opin HIV-AIDS. 2010; 5: 391-396, each of which is incorporated herein by reference.

As to adenovirus vectors useful in the practice of the invention, mention is made of U.S. Pat. No. 6,955,808. The adenovirus vector used can be selected from the group consisting of the Ad5, Ad35, Ad11, C6, and C7 vectors. The sequence of the Adenovirus 5 (“Ad5”) genome has been published. (Chroboczek, J., Bieber, F., and Jacrot, B. (1992) The Sequence of the Genome of Adenovirus Type 5 and Its Comparison with the Genome of Adenovirus Type 2, Virology 186, 280-285; the contents if which is hereby incorporated by reference). Ad35 vectors are described in U.S. Pat. Nos. 6,974,695, 6,913,922, and 6,869,794. Ad11 vectors are described in U.S. Pat. No. 6,913,922. C6 adenovirus vectors are described in U.S. Pat. Nos. 6,780,407; 6,537,594; 6,309,647; 6,265,189; 6,156,567; 6,090,393; 5,942,235 and 5,833,975. C7 vectors are described in U.S. Pat. No. 6,277,558. Adenovirus vectors that are E1-defective or deleted, E3-d elective or deleted, and/or E4˜defective or deleted may also be used. Certain adenoviruses having mutations in the E1 region have improved safety margin because E1-defective adenovirus mutants are replication-defective in non-permissive cells, or, at the very least, are highly attenuated. Adenoviruses having mutations in the E3 region may have enhanced the immunogenicity by disrupting the mechanism whereby adenovirus down-regulates MHC class I molecules. Adenoviruses having E4 mutations may have reduced immunogenicity of the adenovirus vector because of suppression of late gene expression. Such vectors may be particularly useful when repeated re-vaccination utilizing the same vector is desired. Adenovirus vectors that are deleted or mutated in E1, E3, E4, E1 and E3, and E1 and E4 can be used in accordance with the present invention. Furthermore, “gutless” adenovirus vectors, in which ail viral genes are deleted, can also be used in accordance with the present invention. Such vectors require a helper virus for their replication and require a special human 293 cell line expressing both E1a and Cre, a condition that does not exist in natural environment. Such “gutless” vectors are non-immunogenic and thus the vectors may be inoculated multiple times for re-vaccination. The “gutless” adenovirus vectors can be used for insertion of heterologous inserts/genes such as the transgenes of the present invention, and can even be used for co-delivery of a large number of heterologous inserts/genes.

As to lentivirus vector systems useful in the practice of the invention, mention is made of U.S. Pat. Nos. 6,428,953, 6,165,782, 6,013,516, 5,994,136, 6,312,682, and 7,198,784, and documents cited therein.

With regard to AAV vectors useful in the practice of the invention, mention is made of U.S. Pat. Nos. 5,658,785, 7,115,391, 7,172,893, 6,953,690, 6,936,466, 6,924,128, 6,893,865, 6,793,926, 6,537,540, 6,475,769 and 6,258,595, and documents cited therein.

Another vector is BCG (Bacille Calmette Guerin). BCG vectors are described in Stover et al. (Nature 351:456-460 (1991)). A wide variety of other vectors useful for therapeutic administration or immunization of the peptides of the invention, e.g., Salmonella typhi vectors and the like, is apparent to those skilled in the art from the description herein,

Vectors can be administered so as to have in vivo expression and response akin to doses and/or responses elicited by antigen administration

In some embodiments, a means of administering nucleic acids encoding the peptide of the invention uses minigene constructs encoding multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, MEW presentation of CTL epitopes may be improved by including synthetic (e.g. poly-alanine) or naturally-occurring flanking sequences adjacent to the CTL epitopes.

The minigene sequence is converted to DNA by assembling oligonucleotides that encode the plus and minus strands of the minigene. Overlapping oligonucleotides (30-100 bases long) are synthesized, phosphorylated, purified and annealed under appropriate conditions using well known techniques. The ends of the oligonucleotides are joined using T4 DNA ligase. This synthetic minigene, encoding the CTL epitope polypeptide, can then cloned into a desired expression vector.

Standard regulatory sequences well known to those of skill in the art are included in the vector to ensure expression in the target cells. Several vector elements are required: a promoter with a down-stream cloning site for minigene insertion; a polyadenylation signal for efficient transcription termination; an E, coli origin of replication; and an E, coli selectable marker (e.g. ampicillin or kanamycin resistance). Numerous promoters can be used for this purpose, e.g., the human cytomegalovirus (hCMV) promoter. See, U.S. Pat. Nos. 5,580,859 and 5,589,466 for other suitable promoter sequences.

Additional vector modifications may be desired to optimize minigene expression and immunogenicity. In some cases, introns are required for efficient gene expression, and one or more synthetic or naturally-occurring introns could be incorporated into the transcribed region of the minigene. The inclusion of mRNA stabilization sequences can also be considered for increasing minigene expression. It has recently been proposed that immuno stimulatory sequences (ISSs or CpGs) play a role in the immunogenicity of DNA′ vaccines. These sequences could be included in the vector, outside the minigene coding sequence, if found to enhance immunogenicity.

In some embodiments, a bicistronic expression vector, to allow production of the minigene-encoded epitopes and a second protein included to enhance or decrease immunogenicity can be used. Examples of proteins or polypeptides that could beneficially enhance the immune response if co-expressed include cytokines (e.g., IL2, 11,12, GM-CSF), cytokine-inducing molecules (e.g. LeIF) or costimulatory molecules. Helper (HTL) epitopes could be joined to intracellular targeting signals and expressed separately from the CTL epitopes. This would allow direction of the HTL epitopes to a cell compartment different than the CTL epitopes. If required, this could facilitate more efficient entry of HTL epitopes into the MHC class II pathway, thereby improving CTL induction. In contrast to CTL induction, specifically decreasing the immune response by co-expression of immunosuppressive molecules (e.g. TGF-β) may be beneficial in certain diseases.

Once an expression vector is selected, the minigene is cloned into the polylinker region downstream of the promoter. This plasmid is transformed into an appropriate E. coli strain, and DNA is prepared using standard techniques. The orientation and DNA sequence of the minigene, as well as all other elements included in the vector are confirmed using restriction mapping and DNA sequence analysis. Bacterial cells harboring the correct plasmid can be stored as a master cell bank and a working cell bank.

Purified plasmid DNA can be prepared for injection using a variety of formulations. The simplest of these is reconstitution of lyophilized DNA in sterile phosphate-buffer saline (PBS). A variety of methods have been described, and new techniques may become available. As noted herein, nucleic acids are conveniently formulated with cationic lipids. In addition, glycolipids, fusogenic liposomes, peptides and compounds referred to collectively as protective, interactive, non-condensing (P1NC) could also be complexed to purified plasmid DNA to influence variables such as stability, intramuscular dispersion, or trafficking to specific organs or cell types.

Target cell sensitization can be used as a functional assay for expression and MHC class I presentation of minigene-encoded CTL epitopes. The plasmid DNA is introduced into a mammalian cell line that is suitable as a target for standard CTL chromium release assays. The transfection method used is dependent on the final formulation. Electroporation can be used for “naked” DNA, whereas cationic lipids allow direct in vitro transfection. A plasmid expressing green fluorescent protein (GFP) can be co-transfected to allow enrichment of transfected cells using fluorescence activated cell sorting (FACS). These cells are then chromium-51 labeled and used as target cells for epitope-specific CTL lines. Cytolysis, detected by 51 Cr release, indicates production of MHC presentation of mini gene-encoded CTL epitopes.

In vivo immunogenicity is a second approach for functional testing of minigene DNA formulations. Transgenic mice expressing appropriate human MHC molecules are immunized with the DNA product. The dose and route of administration are formulation dependent (e.g. IM for DNA in PBS, IP for lipid-complexed DNA). Twenty-one days after immunization, splenocytes are harvested and restimulated for 1 week in the presence of peptides encoding each epitope being tested. These effector cells (CTLs) are assayed for cytolysis of peptide-loaded, chromium-51 labeled target cells using standard techniques. Lysis of target cells sensitized by MHC loading of peptides corresponding to minigene-encoded epitopes demonstrates DNA vaccine function for in vivo induction of CTLs.

Peptides may be used to elicit CTL ex vivo, as well. The resulting CTL, can be used to treat chronic tumors in patients in need thereof that do not respond to other conventional forms of therapy, or does not respond to a peptide vaccine approach of therapy. Ex vivo CTL responses to a particular tumor antigen are induced by incubating in tissue culture the patient's CTL precursor cells (CTLp) together with a source of antigen-presenting cells (APC) and the appropriate peptide. After an appropriate incubation time (typically 1-4 weeks), in which the CTLp are activated and mature and expand into effector CTL, the cells are infused back into the patient, where they destroy their specific target cell (i.e., a tumor cell). In order to optimize the in vitro conditions for the generation of specific cytotoxic T cells, the culture of stimulator cells are maintained in an appropriate serum-free medium.

Prior to incubation of the stimulator cells with the cells to be activated, e.g., precursor CD 8+ cells, an amount of antigenic peptide is added to the stimulator cell culture, of sufficient quantity to become loaded onto the human Class I molecules to be expressed on the surface of the stimulator cells. In the present invention, a sufficient amount of peptide is an amount that allows about 200, or 200 or more, human Class I MHC molecules loaded with peptide to be expressed on the surface of each stimulator cell. In some embodiments, the stimulator cells are incubated with >2 μg/ml peptide. For example, the stimulator cells are incubated with >3, 4, 5, 10, 15, or more μg/ml peptide.

Resting or precursor CD8+ cells are then incubated in culture with the appropriate stimulator cells for a time period sufficient to activate the CD8+ cells. In some embodiments, the CD8+ cells are activated in an antigen-specific manner. The ratio of resting or precursor CD8+(effector) cells to stimulator cells may vary from individual to individual and may further depend upon variables such as the amenability of an individual's lymphocytes to culturing conditions and the nature and severity of the disease condition or other condition for which the within-described treatment modality is used. The lymphocyte: stimulator cell ratio can be in the range of about 30:1 to 300:1, The effector/stimulator culture may be maintained for as long a time as is necessary to stimulate a therapeutically useable or effective number of CD8+ cells.

The induction of CTL in vitro requires the specific recognition of peptides that are bound to allele specific MHC class I molecules on APC. The number of specific MHC/peptide complexes per APC is crucial for the stimulation of CTL, particularly in primary immune responses. While small amounts of peptide/WIC complexes per cell are sufficient to render a cell susceptible to lysis by CTL, or to stimulate a secondary CTL response, the successful activation of a CTL precursor (pCTL) during primary response requires a significantly higher number of MHC/peptide complexes. Peptide loading of empty major histocompatibility complex molecules on cells allows the induction of primary cytotoxic T lymphocyte responses.

Since mutant cell lines do not exist for every human MHC allele, it is advantageous to use a technique to remove endogenous MHC-associated peptides from the surface of APC, followed by loading the resulting empty WIC molecules with the immunogenic peptides of interest. The use of non-transformed (non-tumorigenic), non-infected cells, and autologous cells of patients as APC is desirable for the design of CTL induction protocols directed towards development of ex vivo CTL therapies. This application discloses methods for stripping the endogenous MHC-associated peptides from the surface of APC followed by the loading of desired peptides.

A stable MHC class I molecule is a trimeric complex formed of the following elements: 1) a peptide usually of 8-10 residues, 2) a transmembrane heavy polymorphic protein chain which bears the peptide-binding site in its a1 and a2 domains, and 3) a non-covalently associated non-polymorphic light chain, p2microglobuiin. Removing the bound peptides and/or dissociating the p2microglobulin from the complex renders the MHC class I molecules nonfunctional and unstable, resulting in rapid degradation. All MHC class I molecules isolated from PBMCs have endogenous peptides bound to them. Therefore, the first step is to remove all endogenous peptides bound to MHC class I molecules on the APC without causing their degradation before exogenous peptides can be added to them.

Two possible ways to free up MHC class I molecules of bound peptides include lowering the culture temperature from 37° C. to 26° C. overnight to destabilize p2microgiobulin and stripping the endogenous peptides from the cell using a mild acid treatment. The methods release previously bound peptides into the extracellular environment allowing new exogenous peptides to bind to the empty class I molecules. The cold-temperature incubation method enables exogenous peptides to bind efficiently to the MHC complex, but requires an overnight incubation at 26° C. which may slow the cell's metabolic rate, it is also likely that cells not actively synthesizing MHC molecules (e.g., resting PBMC) would not produce high amounts of empty surface WIC molecules by the cold temperature procedure.

Harsh acid stripping involves extraction of the peptides with trifluoroacetic acid, pH 2, or acid denaturation of the immunoaffinity purified class I-peptide complexes. These methods are not feasible for CTL induction, since it is important to remove the endogenous peptides while preserving APC viability and an optimal metabolic slate which is critical for antigen presentation. Mild acid solutions of pH 3 such as glycine or citrate-phosphate buffers have been used to identify endogenous peptides and to identify tumor associated T cell epitopes. The treatment is especially effective, in that only the MHC class I molecules are destabilized (and associated peptides released), while other surface antigens remain intact, including MHC class II molecules. Most importantly, treatment of cells with the mild acid solutions does not affect the cell's viability or metabolic state. The mild acid treatment is rapid since the stripping of the endogenous peptides occurs in two minutes at 4° C. and the APC is ready to perform its function after the appropriate peptides are loaded. The technique is utilized herein to make peptide-specific APCs for the generation of primary antigen-specific CTL. The resulting APC are efficient in inducing peptide-specific CD8+ CTL.

Activated CD8+ cells may be effectively separated from the stimulator cells using one of a variety of known methods. For example, monoclonal antibodies specific for the stimulator cells, for the peptides loaded onto the stimulator cells, or for the CD8+ cells (or a segment thereof).may be utilized to bind their appropriate complementary ligand. Antibody-tagged molecules may then be extracted from the stimulator-effector cell admixture via appropriate means, e.g., via well-known immunoprecipitation or immunoassay methods.

Effective, cytotoxic amounts of the activated CD 8+ cells can vary between in vitro and in vivo uses, as well as with the amount and type of cells that are the ultimate target of these killer cells. The amount can also vary depending on the condition of the patient and should be determined via consideration of ail appropriate factors by the practitioner. About 1×10⁶ to about 1×10¹², about 1×10⁸ to about 1×10¹¹, or about 1×10⁹ to about 1×10¹⁰ activated CD8+ cells can be utilized for adult humans, compared to about 5×10⁶-5×10⁷ cells used in mice.

As discussed herein, the activated CD8+ cells are harvested from the cell culture prior to administration of the CD8+ cells to the individual being treated. It is important to note, however, that unlike other present and proposed treatment modalities, the present method uses a cell culture system that is not tumorigenic. Therefore, if complete separation of stimulator cells and activated CD8+ cells are not achieved, there is no inherent danger known to be associated with the administration of a small number of stimulator cells, whereas administration of mammalian tumor-promoting cells may be extremely hazardous.

Methods of re-introducing cellular components are known in the art and include procedures such as those exemplified in U.S. Pat. No. 4,844,893 to Honsik, et al. and U.S. Pat. No. 4,690,915 to Rosenberg. For example, administration of activated CD8+ cells via intravenous infusion is appropriate.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning; A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Wei, 1996); “Gene Transfer Vectors for Mammalian Cells” (Miller and Calos, 1987); “Current Protocols in Molecular Biology” (Ausubel, 1987); “PGR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments are discussed in the sections that follow.

### VIII. Therapeutic Methods

The present invention provides methods of inducing a neoplasia/tumor specific immune response in a subject, vaccinating against a neoplasia/tumor, treating and or alleviating a symptom of cancer in a subject by administering the subject a neoplasia vaccine or a neoantigenic peptide or composition of the invention and at least one inhibitor, such as a checkpoint inhibitor or CD40 agonist.

In particular, the present invention is directed to methods of treating or preventing a neoplasia comprising the steps of administering to a subject (a) a neoplasia vaccine or immunogenic composition, and (b) at least one inhibitor, such as a checkpoint inhibitor or CD40 agonist.

According to the invention, the herein-described neoplasia vaccine or immunogenic composition may be used for a patient that has been diagnosed as having cancer, or at risk of developing cancer.

The described combination of the invention is administered in an amount sufficient to induce a CTL response.

The vaccine or immunogenic composition comprising neoantigenic peptides may be administered to a subject for the treatment of a condition. The subject may be administered one or more inhibitors, such as a checkpoint inhibitor or CD40 agonist, in addition to the neoantigenic peptides. Administration of the one or more inhibitors, such as a checkpoint inhibitor or CD40 agonist, may be performed before the administration of the neoantigenic peptides. In some embodiments, more than one inhibitor, such as a checkpoint inhibitor or CD40 agonist, is being administered to the patient. In such cases, one inhibitor, such as a checkpoint inhibitor or CD40 agonist, may be administered before the administration of the neoantigen peptides. For instance, in a case where a combination of neoantigenic peptides, nivolumab and another inhibitor, such as a checkpoint inhibitor or CD40 agonist, is being administered to the patient, a starting dose of nivolumab may be administered to a patient before the neoantigenic peptides are administered.

Patients may be screened before administration and after the administration of the compositions described herein. Patients may undergo screening assessments that document historical health status as well as their current and future health status in general and as related to their underlying disease. The screening assessments may include tests like vital signs (including diastolic and systolic blood pressure, heart rate, temperature, weight, and height), electrocardiograms, symptom directed physical exam, hematology (including hematocrit, hemoglobin, RBC count, WBC count with differential, and platelet count), chemistry (including tests for glucose, urea nitrogen, creatinine, sodium, potassium, calcium, total and direct bilirubin, AST, ALT, alkaline phosphatase, lactate dehydrogenase (LDH), and adrenocorticotropic hormone), liver function tests (such as detecting levels of AST, ALT, total and direct bilirubin), pregnancy testing, CT or MRI, surgical or core needle biopsy of a primary or metastatic tumor site for DNA and RNA sequencing, immunological analysis. Biopsies may be used to evaluate the presence of T-cell infiltrates in the tumor and their localization with respect to tumor margins by tests like immunohistochemistry, western blot analysis, RNA and DNA analysis. The presence of tumor-associated macrophages and DCs within the tumor micro-environment may also be evaluated. The list of markers for analysis may include, but not be limited to, the following: CD3, CD4, CD8, CD45RO, PD-L1, PD-1, FoxP3, Granzyme B, Perforin, CD68, CD163, MHC Class I, MHC Class II, CD83 AND CD11b.

Immune response parameters may be analyzed for changes over time from baseline levels before the administration of the treatments and may include summaries of characterization of nucleic acids (e.g., DNA mutations, transcript abundance), histopathology, and immune cell analyses in tissues obtained at the Pretreatment, Pre-vaccination Treatment, and Vaccination Treatment phases as well as at the time of preliminary assessment. Reporting the test results may include tables depicting shifts from earlier time points in order to compare changes in immune parameters. Descriptive statistics and frequency distributions may be used as appropriate. Immunological analyses may include summaries for CD8+ and CD4+ T-cell response measured by ex vivo IFN-γ ELISpot and assessed through spot counts. Analyses can be used to assess changes from pretreatment to pre-vaccination treatment to vaccination treatment to preliminary assessment. Reporting may include medians and inter-quartile ranges, as well as tables depicting shifts from earlier time points for each patient. Additionally, nonparametric tests (e.g., Wilcoxon signed-rank test) may be used to determine differences in the ELISpot data between time points as appropriate. Fold changes in biomarkers measured on a continuous scale may be summarized and compared across response categories using the Wilcoxon rank-sum test between treatment arms for every cohort. For multi-gene assays, genes may be grouped into analysis sets to characterize biological functions of the cells as appropriate.

Tests and results may include a detailed characterization of the phenotype and abundance of antigen-specific T cells, both in the periphery and in the tumor microenvironment. The abundance of regulatory cells such as regulatory T cells or myeloid-derived suppressor cells, and T-cell recognition, activation, and cytotoxicity may also be evaluated with PBMCs and tumor cells. Additionally, ex vivo induction of neoantigen T-cell responses may also be performed on peripheral blood and leukopheresis samples. Presence of circulating tumor DNA (ctDNA) and vaccine-specific antibody responses may be evaluated after treatment with the compositions described herein.

### IX. Additional Therapies

The tumor specific neoantigen peptides and pharmaceutical compositions described herein can also be administered in further combination with another agent, for example a therapeutic agent. In certain embodiments, the additional agents can be, but are not limited to, chemotherapeutic agents, anti-angiogenesis agents and agents that reduce immune-suppression.

The neoplasia vaccine or immunogenic composition and one or more inhibitors, such as a checkpoint inhibitor or CD40 agonist, can be administered before, during, or after administration of the additional agent. In embodiments, the neoplasia vaccine or immunogenic composition and/or one or more inhibitors, such as a checkpoint inhibitor or CD40 agonist, are administered before the first administration of the additional agent. In other embodiments, the neoplasia vaccine or immunogenic composition and/or one or more inhibitors, such as a checkpoint inhibitor or CD40 agonist, are administered after the first administration of the additional therapeutic agent (e.g., 1, 2, 3, 4, 5, 6, 7, 8, 9, 10, 11, 12, 13, 14 or more days). In embodiments, the neoplasia vaccine or immunogenic composition and one or more inhibitors, such as a checkpoint inhibitor or CD40 agonist, are administered simultaneously with the first administration of the additional therapeutic agent.

The therapeutic agent is for example, a chemotherapeutic or biotherapeutic agent, radiation, or immunotherapy. Any suitable therapeutic treatment for a particular cancer may be administered. Examples of chemotherapeutic and biotherapeutic agents include, but are not limited to, an angiogenesis inhibitor, such ashydroxy angiostatin K 1-3, DL-a-Difluoromethyl-ornithine, endostatin, fumagillin, genistein, minocycline, staurosporine, and thalidomide; a DNA intercaltor/cross-linker, such as Bleomycin, Carboplatin, Carmustine, Chlorambucil, Cyclophosphamide, cis-Diammineplatinum(II) dichloride (Cisplatin), Melphalan, Mitoxantrone, and Oxaliplatin; a DNA synthesis inhibitor, such as (±)-Amethopterin (Methotrexate), 3-Amino-1,2,4-beiizotriazine 1,4-dioxide, Aminopterin, Cytosine β-D-arabinofuranoside, 5-Fluoro-5′˜ deoxyuridine, 5-Flurorouracil, Ganciclovir, Hydroxyurea, and Mitomycin C; a DNA-RNA transcription regulator, such as Actinomycin D, Daunorubicin, Doxorubicin, Homoharringtonine, and Idarubicin; an enzyme inhibitor, such as S(+)-Camptothecin, Curcumin, (−)-Deguelin, 5,6-Dichlorobenzimidazole 1-β-D-ribofuranoside, Etoposide, Formestane, Fostriecin, Hispidin, 2-Imino-1-imidazole-dineacetic acid (Cyclocreatine), Mevinolin, Trichostatin A, Tyrphostin AG 34, and Tyrphostin AG 879; a gene regulator, such as 5-Aza-2′-deoxycytidine, 5-Azacytidine, Cholecalciferol (Vitamin D3), 4-Hydroxytamoxifen, Melatonin, Mifepristone, Raloxifene, all trans-Retinal (Vitamin A aldehyde), Retinoic acid all trans (Vitamin A acid), 9-cis-Retinoic Acid, 13-cis-Retinoic acid, Retinol (Vitamin A), Tamoxifen, and Troglitazone; a microtubule inhibitor, such as Colchicine, docetaxel, Dolastatins 15, Nocodazole, Paclitaxel, Podophyllotoxin, Rhizoxin, Vinblastine, Vincristine, Vindesine, and Vinorelbine (Navelbine); and an unclassified therapeutic agent, such as 17-(Allylamino)-1 7-demethoxygeldanamycin, 4-Amino-1,8-naphthalimide, Apigenin, Brefeldin A, Cimetidine, Dichloromethylene-diphosphonic acid, Leuprolide (Leuprorelin), Luteinizing Hormone-Releasing Hormone, Pifithrin-α, Rapamycin, Sex hormone-binding globulin, Thapsigargin, and Urinary trypsin inhibitor fragment (Bikunin). The therapeutic agent may be altretamine, amifostine, asparaginase, capecitabine, cladribine, cisapride, cytarabine, dacarbazine (DT1C), dactinomycin, dronabinol, epoetin alpha, filgrastim, fludarabine, gemcitabine, granisetron, ifosfamide, irinotecan, lansoprazole, levamisole, leucovorin, megestrol, mesna, metoclopramide, mitotane, omeprazole, ondansetron, pilocarpine, prochloroperazine, or topotecan hydrochloride. The therapeutic agent may be a monoclonal antibody such as rituximab (Rituxan®), alemtuzumab (Campath®), Bevacizumab (Avastin®), Cetuximab (Erbitux®), panitumumab (Vectibix®), and trastuzumab (Herceptin®), Vemurafenib (Zelboraf®) imatinib mesylate (Gleevec®), erlotinib (Tarceva®), gefitinib (Iressa®), Vismodegib (Erivedge™), 90Y-ibritumomab tiuxetan, 1311-tositumomab, ado-trastuzumab emtansine, lapatinib (Tykerb®), pertuzumab (Perjeta™), ado-trastuzumab emtansine (Adcyla™), regorafenib (Stivarga®), sunitinib (Sutent®), Denosumab (Xgeva®), sorafenib (Nexavar®), pazopanib (Votrient®), axitinib (Ini Ta®), dasatinib (Sprycel®), nilotinib (Tasigna®), bosutinib (Bosulif®), ofatumumab (Arzerra®), obinutuzumab (Gazyva™), ibrutinib (Imbruvica™), idelalisib (Zydelig®), crizotinib (Xalkori®), erlotinib (Tarceva®), afatimb dimaleate (Giiotrif®), ceritinib (LDK378/Zykadia), Tositumomab and 1311-tositumomab (Bexxar®), ibritumomab tiuxetan (Zevalin®), brentuximab vedotin (Adcetris®), bortezomib (Velcade®), siltuximab (Sylvant™), trametinib (Mekinist®), dabrafenib (Tafmlar®), pembrolizimiab (Keytruda®), carfilzomib (Kyprolis®), Ramucirumab (Cyramza™) Cabozantinib (Cometriq™), vandetanib (Caprelsa®), Optionally, the therapeutic agent is a neoantigen. The therapeutic agent may be a cytokine such as interferons (INFs), interleukins (ILs), or hematopoietic growth factors. The therapeutic agent may be INF-α, IL-2, Aldesleukin, IL-2, Erythropoietin, Granulocyte-macrophage colony-stimulating factor (GM-CSF) or granulocyte colony-stimulating factor. The therapeutic agent may be a targeted therapy such as toremifene (Fareston®), fulvestrant (Faslodex®), anastrozole (Arimidex®), exemestane (Aromasin®), letrozole (Femara®), ziv-aflibercept (Zaltrap®), alitretinoin (Panretin®), temsirolimus (Torisel®), Tretinoin (Vesanoid®), denileukin diftitox (Ontak®), vorinostat (Zoiinza®), romidepsin (Istodax®), bexarotene (Targretin®), pralatrexate (Foiotyn®), lenaliomide (Revlimid®), belinostat (Beleodaq™), lenaliomide (Revlimid®), pomalidomide (Pomalyst®), Cabazitaxel (Jevtana®), enzaluiamide (Xtandi®), abiraterone acetate (Zytiga®), radium 223 chloride (Xofigo®), or everolimus (Afinitor®). Additionally, the therapeutic agent may be an epigenetic targeted drug such as HDAC inhibitors, kinase inhibitors, DNA methyltransferase inhibitors, histone demethylase inhibitors, or histone methylation inhibitors. The epigenetic drugs may be Azacitidine (Vidaza), Decitabine (Dacogen), Vorinostat (Zoiinza), Romidepsin (Istodax), or Ruxolitinib (Jakafi). For prostate cancer treatment, a chemotherapeutic agent with which anti-CTLA-4 can be combined is paclitaxel (TAXOL).

In certain embodiments, the one or more additional agents are one or more anti-glucocorticoid-induced tumor necrosis factor family receptor (GITR) agonistic antibodies. GITR is a costimulatory molecule for T lymphocytes, modulates innate and adaptive immune system and has been found to participate in a variety of immune responses and inflammatory processes. GITR was originally described by Nocentini et al. after being cloned from dexamethasone-treated murine T cell hybridomas (Nocentini et al, Proc Natl Acad Sci USA 94:6216-6221.1997). Unlike CD28 and CTLA-4, GITR has a very low basal expression on naive CD4+ and CD8+ T cells (Ronchetti et al. Eur J Immunol 34:613-622. 2004). The observation that GITR stimulation has immunostimulatory effects in vitro and induced autoimmunity in vivo prompted the investigation, of the antitumor potency of triggering this pathway. A review of Modulation Of CTLA4 And GITR For Cancer Immunotherapy can be found in Cancer Immunology and Immunotherapy (Avogadri et al. Current Topics in Microbiology and Immunology 344. 2011). Other agents that can contribute to relief of immune suppression include checkpoint inhibitors targeted at another member of the CD28/CTLA4 Ig superfamily such as BTLA, LAG 3, ICOS, PDL1 or J (Page et a, Annual Review of Medicine 65:27 (2014)). In further additional embodiments, the inhibitor is targeted at a member of the TNFR superfamily such as CD40, OX40, CD137, GITR, CD27 or TIM-3. In some cases the inhibitor is an inhibitory antibody or similar molecule. In other cases, the inhibitor is agonist for the target (e.g., CD40); examples of this class include the stimulatory targets OX40 and GITR.

In certain embodiments, the one or more additional agents are synergistic in that they increase immunogenicity after treatment. In one embodiment the additional agent allows for lower toxicity and/or lower discomfort due to lower doses of the additional therapeutic agents or any components of the combination therapy described herein. In another embodiment the additional agent results in longer lifespan due to increased effectiveness of the combination therapy described herein. Chemotherapeutic treatments that enhance the immunological response in a patient have been reviewed (Zitvogel et al., Immunological aspects of cancer chemotherapy. Nat Rev Immunol. 2008 Jan.; 8(1):59-73). Additionally, chemotherapeutic agents can be administered safely with immunotherapy without inhibiting vaccine specific T-cell responses (Perez et al., A new era in anticancer peptide vaccines. Cancer May 2010). In one embodiment the additional agent is administered to increase the efficacy of the combination therapy described herein. In one embodiment the additional agent is a chemotherapy treatment. In one embodiment low doses of chemotherapy potentiate delayed-type hypersensitivity (DTH) responses. In one embodiment the chemotherapy agent targets regulatory T-cells. In one embodiment cyclophosphamide is the therapeutic agent. In one embodiment cyclophosphamide is administered prior to vaccination. In one embodiment cyclophosphamide is administered as a single dose before vaccination (Walter et al, Multipeptide immune response to cancer vaccine IMA901 after single-dose cyclophosphamide associates with longer patient survival. Nature Medicine; 18:8 2012). In another embodiment cyclophosphamide is administered according to a metronomic program, where a daily dose is administered for one month (Ghiringhelli et al, Metronomic cyclophosphamide regimen selectively depletes CD4+CD25+ regulatory T cells and restores T and NK effector functions in end stage cancer patients. Cancer Immunol Immunother 2007 56:641-648). In another embodiment taxanes are administered before vaccination to enhance T-cell and NK-cell functions (Zitvogel et al, 2008). In another embodiment a low dose of a chemotherapeutic agent is administered with the combination therapy described herein. In one embodiment the chemotherapeutic agent is estramustine. In one embodiment the cancer is hormone resistant prostate cancer. A >50% decrease in serum prostate specific antigen (PSA) was seen in 8.7% of advanced hormone refractory prostate cancer patients by personalized vaccination alone, whereas such a decrease was seen in 54% of patients when the personalized vaccination, was combined with a low dose of estramustine (I ton et al., Personalized peptide vaccines: A new therapeutic modality for cancer. Cancer Sci 2006; 97: 970-976). In another embodiment glucocorticoids are not administered with or before the combination therapy described herein (Zitvogef et al., 2008). In another embodiment glucocorticoids are administered after the combination therapy described herein. In another embodiment Gemcitabine is administered before, simultaneously, or after the combination therapy described herein to enhance the frequency of tumor specific CTL precursors (Zitvogel et al., 2008). In another embodiment 5-ftuorouracil is administered with the combination therapy described herein as synergistic effects were seen with a peptide based vaccine (Zitvogel et al., 2008). In another embodiment an inhibitor of Braf, such as Vemurafenib, is used as an additional agent. Braf inhibition has been shown to be associated with an increase in melanoma antigen expression and T-cell infiltrate and a decrease in immunosuppressive cytokines in tumors of treated patients (Frederick et al, BRAF inhibition is associated with enhanced melanoma antigen expression and a more favorable tumor microenvironment in patients with metastatic melanoma. Clin Cancer Res. 2013; 19:1225-1231). In another embodiment an inhibitor of tyrosine kinases is used as an additional agent. In one embodiment the tyrosine kinase inhibitor is used before vaccination with the combination therapy described herein. In one embodiment the tyrosine kinase inhibitor is used simultaneously with the combination therapy described herein. In another embodiment the tyrosine kinase inhibitor is used to create a more immune permissive environment. In another embodiment the tyrosine kinase inhibitor is sunitinib or matinib mesylate. It has previously been shown that favorable outcomes could be achieved with sequential administration of continuous daily dosing of sunitinib and recombinant vaccine (Farsaci et al., Consequence of dose scheduling of sunitinib on host immune response elements and vaccine combination therapy. Int J Cancer; 130: 1948-1959). Sunitinib has also been shown to reverse type-1 immune suppression using a daily dose of 50 mg/day (Finke et al., Sunitinib Reverses Type-1 Immune Suppression and Decreases T-Regulatory Cells in Renal Cell Carcinoma Patients. Clin Cancer Res 2008; 14(20)). In another embodiment targeted therapies are administered in combination with the combination therapy described herein. Doses of targeted therapies have been described previously (Alvarez, Present and future evolution of advanced breast cancer therapy. Breast Cancer Research 2010, 12 (Suppl 2):S1). In another embodiment temozolomide is administered with the combination therapy described herein. In one embodiment temozolomide is administered at 200 mg day for 5 days every fourth week of a combination therapy with the combination therapy described herein. Results of a similar strategy have been shown to have low toxicity (Kyte et al., Telomerase Peptide Vaccination Combined with Temozolomide: A Clinical Trial in Stage IV Melanoma Patients. Clin Cancer Res; 17(13) 2011). In another embodiment the combination therapy is administered with an additional therapeutic agent that results in lymphopenia. In one embodiment the additional agent is temozolomide. An immune response can still be induced under these conditions (Sampson et al., Greater chemotherapy-induced lymphopenia enhances tumor-specific immune responses that eliminate EGFRvIII-expressing tumor cells in patients with glioblastoma. Neuro-Oncology 13(3):324-333, 2011).

The herein-described compositions and methods may be used on patients in need thereof with any cancer according to the general flow process comprising subject identification, collecting informed consent and pre-screening the patients. Patients can then undergo an assessment of the specific type of cancer and the mutations causing the cancer. The nucleic acid material collected from a patient may be used for exome sequencing (tumor and normal tissue), RNA sequencing for the preparation of specific personalized vaccine. Patients in need thereof may receive a series of priming vaccinations with a mixture of personalized tumor-specific peptides. Additionally, over a 4 week period the priming may be followed by two boosts during a maintenance phase. All vaccinations are subcutaneously delivered. The vaccine or immunogenic composition is evaluated for safety, tolerability, immune response and clinical effect in patients and for feasibility of producing vaccine or immunogenic composition and successfully initiating vaccination within an appropriate time frame. The first cohort can consist of 5 patients, and after safety is adequately demonstrated, an additional cohort of 10 patients may be enrolled. Peripheral blood is extensively monitored for peptide-specific T-cell responses and patients are followed for up to two years to assess disease recurrence.

### X. Administering Combination Therapy Consistent with Standard of Care

In another aspect, the combination therapy described herein provides selecting the appropriate point to administer the combination therapy in relation to and within the standard of care for the cancer being treated for a patient in need thereof. The studies described herein show that the combination therapy can be effectively administered even within the standard of care that includes surgery, radiation, or chemotherapy. The standards of care for the most common cancers can be found on the website of National Cancer Institute (http://www.cancer.gov/eancertopies). The standard of care is the current treatment that is accepted by medical experts as a proper treatment for a certain type of disease and that is widely used by healthcare professionals. Standard or care is also called best practice, standard medical care, and standard therapy. Standards of Care for cancer generally include surgery, lymph node removal, radiation, chemotherapy, targeted therapies, antibodies targeting the tumor, and immunotherapy. Immunotherapy can include checkpoint blockers (CBP), chimeric antigen receptors (CAILs), and adoptive T-cell therapy. The combination therapy described herein can be incorporated within the standard of care. The combination therapy described herein may also be administered where the standard of care has changed due to advances in medicine.

Incorporation of the combination therapy described herein may depend on a treatment step in the standard of care that can lead to activation of the immune system. Treatment steps that can activate and function synergistically with the combination therapy have been described herein. The therapy can be advantageously administered simultaneously or after a treatment that activates the immune system.

Incorporation of the combination therapy described herein may depend on a treatment step in the standard of care that causes the immune system to be suppressed. Such treatment steps may include irradiation, high doses of alkylating agents and/or methotrexate, steroids such as glucosteroids, surgery, such as to remove the lymph nodes, imatinib mesylate, high doses of TNF, and taxanes (Zitvogel et al., 2008). The combination therapy may be administered before such steps or may be administered after.

In one embodiment the combination therapy may be administered after bone marrow transplants and peripheral blood stem cell transplantation. Bone marrow transplantation and peripheral blood stem cell transplantation are procedures that restore stem cells that were destroyed by high doses of chemotherapy and/or radiation therapy. After being treated with high-dose anticancer drugs and/or radiation, the patient receives harvested stem cells, which travel to the bone marrow and begin to produce new blood cells. A “mini-transplant” uses lower, less toxic doses of chemotherapy and/or radiation to prepare the patient for transplant. A “tandem transplant” involves two sequential courses of high-dose chemotherapy and stem cell transplant. In autologous transplants, patients receive their own stem cells. In syngeneic transplants, patients receive stem cells from their identical twin. In allogeneic transplants, patients receive stem cells from their brother, sister, or parent. A person who is not related to the patient (an unrelated donor) also may be used, In some types of leukemia, the graft-versus-tumor (GVT) effect that occurs after allogeneic BMT and PBSCT is crucial to the effectiveness of the treatment. GVT occurs when white blood cells from the donor (the graft) identify the cancer cells that remain in the patient's body after the chemotherapy and/or radiation therapy (the tumor) as foreign and attack them. Immunotherapy with the combination therapy described herein can take advantage of this by vaccinating after a transplant. Additionally, the transferred cells may be presented with neoantigens of the combination therapy described herein before transplantation.

In one embodiment the combination therapy is administered to a patient in need thereof with a cancer that requires surgery. In one embodiment the combination therapy described herein is administered to a patient in need thereof in a cancer where the standard of care is primarily surgery followed by treatment to remove possible micro-metastases, such as breast cancer. Breast cancer is commonly treated by various combinations of surgery, radiation therapy, chemotherapy, and hormone therapy based on the stage and grade of the cancer. Adjuvant therapy for breast cancer is any treatment given after primary therapy to increase the chance of long-term survival. Neoadjuvant therapy is treatment given before primary therapy. Adjuvant therapy for breast cancer is any treatment given after primary therapy to increase the chance of long-term disease-free survival. Primary therapy is the main treatment used to reduce or eliminate the cancer. Primary therapy for breast cancer usually includes surgery, a mastectomy (removal of the breast) or a lumpectomy (surgery to remove the tumor and a small amount of normal tissue around it; a type of breast-conserving surgery). During either type of surgery, one or more nearby lymph nodes are also removed to see if cancer cells have spread to the lymphatic system. When a woman has breast-conserving surgery, primary therapy almost always includes radiation therapy. Even in early-stage breast cancer, cells may break away from the primary tumor and spread to other parts of the body (metastasize). Therefore, doctors give adjuvant therapy to kill any cancer cells that may have spread, even if they cannot be detected by imaging or laboratory tests.

In one embodiment the combination therapy is administered consistent with the standard of care for Ductal carcinoma in situ (DCIS). Care for this breast cancer type comprises: 1. Breast-conserving surgery and radiation therapy with or without tamoxifen. 2. Total mastectomy with or without tamoxifen. 3. Breast-conserving surgery without radiation therapy.

The combination therapy may be administered before breast conserving surgery or total mastectomy to shrink the tumor before surgery. In another embodiment the combination therapy can be administered as an adjuvant therapy to remove any remaining cancer cells.

In another embodiment patients diagnosed with stage I, II, IIIA, and Operable IIIC breast cancer are treated with the combination therapy as described herein. Care for this breast cancer type comprises:
1. Local-regional treatment;
   Breast-conserving therapy (lumpectomy, breast radiation, and surgical staging of the axilla).
   Modified radical mastectomy (removal of the entire breast with level I-II axillary dissection) with or without breast reconstruction.
   Sentinel node biopsy.
2. Adjuvant radiation therapy postmastectomy in axillary node-positive tumors:
   For one to three nodes: unclear role for regional radiation (infra/supraclavicular nodes, internal mammary nodes, axillary nodes, and chest wall).
   For more than four nodes or extranodal involvement: regional radiation is advised.
3. Adjuvant systemic therapy

In one embodiment the combination therapy is administered as a neoadjuvant therapy to shrink the tumor. In another embodiment the combination is administered as an adjuvant systemic therapy.

In another embodiment patients diagnosed with inoperable stage IIIB or IIIC or inflammatory breast cancer are treated with the combination therapy as described herein. The standard of care for this breast cancer type is: 1. Multimodality therapy delivered with curative intent is the standard of care for patients with clinical stage IHB disease. 2. Initial surgery is generally limited to biopsy to permit the determination of histology, estrogen-receptor (ER) and progesterone-receptor (PR) levels, and human epidermal growth factor receptor 2 (HER2/neu) overexpression. Initial treatment with anthracycline-based chemotherapy and/or taxane-based therapy is standard. For patients who respond to neoadjuvant chemotherapy, local therapy may consist of total mastectomy with axillary lymph node dissection followed by postoperative radiation therapy to the chest wall and regional lymphatics. Breast-conserving therapy can be considered in patients with a good partial or complete response to neoadjuvant chemotherapy. Subsequent systemic therapy may consist of further chemotherapy. Hormone therapy should be administered to patients whose tumors are ER-positive or unknown. All patients should be considered candidates for clinical trials to evaluate the most appropriate fashion in which to administer the various components of multimodality regimens.

In one embodiment the combination therapy is administered as part of the various components of multimodality regimens. In another embodiment the combination therapy is administered before, simultaneously with, or after the multimodality regimens. In another embodiment the combination therapy is administered based on synergism between the modalities. In another embodiment the combination therapy is administered after treatment with anthracycline-based chemotherapy and/or taxane-based therapy (Zirvogel et al, 2008). Treatment after administering the combination therapy may negatively affect dividing effector T-cells. The combination therapy may also be administered after radiation.

In another embodiment the combination therapy described herein is used in the treatment in a cancer where the standard of care is primarily not surgery and is primarily based on systemic treatments, such as Chronic Lymphocytic Leukemia (CLL).

In another embodiment patients diagnosed with stage I, II, III, and IV Chronic Lymphocytic Leukemia are treated with the combination therapy as described herein. The standard of care for this cancer type is:

1. Observation in asymptomatic or minimally affected patients

### 2. Rituximab

### 3. Ofatumomab

4. Oral alkylating agents with or without corticosteroids
5. Fludarabine, 2-chlorodeoxyadenosine, or pentostatin

### 6. Bendamustine

### 7. Lenalidomide

8. Combination chemotherapy.
Combination chemotherapy regimens include the following:
   Fludarabine plus cyclophosphamide plus rituximab.
   Fludarabine plus rituximab as seen in the CLB-9712 and CLB-9011 trials,
   Fludarabine plus cyclophosphamide versus fludarabine plus cyclophosphamide plus rituximab.
   Pentostatin plus cyclophosphamide plus rituximab as seen in the MAYO-MC0183 trial, for example,
   Ofatumumab plus fludarabine plus cyclophosphamide,
   CVP: cyclophosphamide plus vincristine plus prednisone,
   CHOP: cyclophosphamide plus doxorubicin plus vincristine plus prednisone,
   Fludarabine plus cyclophosphamide versus fludarabine as seen in the E2997 trial [NCT00003764] and the LRF-CLL4 trial, for example,
   Fludarabine plus chlorambucil as seen in the CLB-9011 trial, for example.
      9. Involved-field radiation therapy.

### 10. Alemtuzumab

11. Bone marrow and peripheral stem cell transplantations are under clinical evaluation,

### 12. Ibrutinib

In one embodiment the combination therapy is administered before, simultaneously with or after treatment with Rituximab or Ofatumomab. As these are monoclonal antibodies that target B-cells, treatment with the combination therapy may be synergistic. In another embodiment the combination therapy is administered after treatment with oral alkylating agents with or without corticosteroids, and Fludarabine, 2-chlorodeoxyadenosine, or pentostatin, as these treatments may negatively affect the immune system if administered before. In one embodiment bendamustine is administered with the combination therapy in low doses based on the results for prostate cancer described herein. In one embodiment the combination therapy is administered after treatment with bendamustine.

### XI. Vaccine or Immunogenic Composition Kits and Co-Packaging

In an aspect, the invention provides kits containing any one or more of the elements discussed herein to allow administration of the combination therapy. Elements may be provided individually or in combinations, and may be provided in any suitable container, such as a vial, a bottle, or a tube. In some embodiments, the kit includes instructions in one or more languages, for example in more than one language. In some embodiments, a kit comprises one or more reagents for use in a process utilizing one or more of the elements described herein. Reagents may be provided in any suitable container. For example, a kit may provide one or more delivery or storage buffers. Reagents may be provided in a form that is usable in a particular process, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more of the vectors, proteins and/or one or more of the polynucleotides described herein. The kit may advantageously al low the provision of all elements of the systems of the invention. Kits can involve vector(s) and/or particle(s) and/or nanoparticle(s) containing or encoding RNA(s) for 1-50 or more neoantigen mutations to be administered to an animal, mammal, primate, rodent, etc., with such a kit including instructions for administering to such a eukaryote; and such a kit can optionally include any of the anti-cancer agents described herein. The kit may include any of the components above (e.g. vector(s) and/or particle(s) and/or nanoparticle(s) containing or encoding RNA(s) for 1-50 or more neoantigen mutations, neoantigen proteins or peptides, checkpoint inhibitors, or CD40 agonists) as well as instructions for use with any of the methods of the present invention.

In one embodiment the kit contains at least one vial with an immunogenic composition or vaccine and at least one vial with an anticancer agent. In one embodiment kits may comprise ready to use components that are mixed and ready to administer. In one aspect a kit contains a ready to use immunogenic or vaccine composition and a ready to use anti-cancer agent. The ready to use immunogenic or vaccine composition may comprise separate vials containing different pools of immunogenic compositions. The immunogenic compositions may comprise one vial containing a viral vector or DNA plasmid and the other vial may comprise immunogenic protein. The ready to use anticancer agent may comprise a cocktail of anticancer agents or a single anticancer agent. Separate vials may contain different anti-cancer agents. In another embodiment a kit may contain a ready to use anti-cancer agent and an immunogenic composition or vaccine in a ready to be reconstituted form. The immunogenic or vaccine composition may be freeze dried or lyophilized. The kit may comprise a separate vial with a reconstitution buffer that can be added to the lyophilized composition so that it is ready to be administered. The buffer may advantageously comprise an adjuvant or emulsion according to the present invention. In another embodiment the kit may comprise a ready to reconstitute anticancer agent and a ready to reconstitute immunogenic composition or vaccine. In this aspect both may be lyophilized. In this aspect separate reconstitution buffers for each may be included in the kit. The buffer may advantageously comprise an adjuvant or emulsion according to the present invention. In another embodiment the kit may comprise single vials containing a dose of immunogenic composition and anti-cancer agent that are administered together. In another aspect multiple vials are included so that one vial is administered according to a treatment timeline. One vial may only contain the anti-cancer agent for one dose of treatment, another may contain both the anti-cancer agent and immunogenic composition for another dose of treatment and one vial may only contain the immunogenic composition for yet another dose. In a further aspect the vials are labeled for their proper administration to a patient in need thereof. The immunogen or anti-cancer agents of any embodiment may be in a lyophilized form, a dried form or in aqueous solution as described herein. The immunogen may be a live attenuated virus, protein, or nucleic acid as described herein.

In one embodiment the anticancer agent is one that enhances the immune system to enhance the effectiveness of the immunogenic composition or vaccine. In an embodiment the anti-cancer agent is an inhibitor, such as a checkpoint inhibitor or CD40 agonist. In another embodiment the kit contains multiple vials of immunogenic compositions and anti-cancer agents to be administered at different time intervals along a treatment plan. In another embodiment the kit may comprise separate vials for an immunogenic composition for use in priming an immune response and another immunogenic composition to be used for boosting. In one aspect the priming immunogenic composition could be DNA or a “viral, vector and the boosting immunogenic composition may be protein. Either composition may be lyophilized or ready for administering. In another embodiment different cocktails of anti-cancer agents containing at least one anticancer agent are included in different vials for administration in a treatment plan.

Although the present invention and its advantages have been described in detail, it should be understood that various changes, substitutions and alterations can be made herein without departing from the spirit and scope of the invention as defined in the appended claims.

The present invention is further illustrated in the following Examples which are given for illustration purposes only and are not intended to limit the invention in any way.

### EXAMPLES

### Example 1: Treatment of Melanoma Patients

According to the Surveillance, Epidemiology, and End Results (SEER) program's data, approximately 76,100 individuals were diagnosed with melanoma and an estimated 9710 died from the disease in 2014 in the United States (US). The incidence of melanoma continues to rise worldwide at approximately 3% per year. Roughly 4% of melanomas are already metastatic at the time of diagnosis. Disseminated, locally advanced, or recurrent melanoma is notoriously unresponsive to standard treatment and is associated with a dismal prognosis, with 5-year survival of the order of 10%-25%. Recent advances with targeted agents that block driver oncogenic mutations such as BRAF^{v600} have shown significant but transient clinical efficacy in patients with unresectable or metastatic melanoma (Flaherty, 2012; Sosman, 2012), whereas immunotherapy using co-stimulatory molecule blockade with monoclonal antibodies (mAbs) such as anti-cytotoxic T-lymphocyte-associated antigen 4 (CTLA-4), programmed cell death protein 1 (PD-1), or programmed death ligand 1 (PD-L1) can lead to durable responses in 15%-40% of melanoma patients (Hodi, 2010; Brahmer, 2012; Topalian, 2012; Topalian, 2014). Nivolumab is approved for the treatment of BRAF^{v600} wild-type and mutant unresectable or metastatic melanoma.

Patients with melanoma are chosen for administering the compositions described herein because this tumor type frequently has a large number of mutations, reflecting its etiology as a carcinogen-induced cancer (Alexandrov, 2013). Immunotherapy with anti-PD-1, ipilimumab, and other immunotherapeutic agents has shown some anti-tumor activity in melanoma; however, many patients do not respond or do not respond robustly. By combining the immune-stimulating effects of a neoantigen-targeted vaccine, the enhanced immune priming from CD40 agonism using APX005M, and the release of immune suppression by an immunomodulator such as nivolumab, a significant improvement in the response rate, the depth of tumor responses, and the durability of responses may be possible due to the synergistic effects of the combination therapies. By combining the immune-stimulating effects of a neoantigen-targeted vaccine, the enhanced immune priming from CTLA4 agonism using ipilimumab, and the release of immune suppression by an immunomodulator such as nivolumab, a significant improvement in the response rate, the depth of tumor responses, and the durability of responses are possible due to the synergistic effects of the combination therapies. Patients with other types of cancer are enrolled in different studies with different regimens of the same compositions.

Such patients have a benefit from treatment with the above mentioned compositions. Patients with advanced or metastatic melanoma are enrolled in a clinical trial to evaluate the safety of administering neoantigens using different regimens as well as in combination with APX005M or ipilimumab with nivolumab. Patients with other types of neoplasia can also benefit from treatment with these compositions.

Patients enrolled in a clinical study are evaluated in a screening and pretreatment period. The Screening Period and Pretreatment Period runs concurrently over approximately 30 days and can be extended by 15 days (to 45 days total) if a repeat biopsy is required. Enrolled patients then undergo surgical or core needle biopsy of a primary or metastatic tumor site for DNA and RNA sequencing, immunological analysis as described herein.

### Example 2: Neoantigenic Peptide Composition Preparation

The neoantigenic peptide vaccine composition is prepared by combining the peptides with an adjuvant such as Poly-ICLC. One instance of the composition comprises:
Neoantigenic peptides: Personalized neoantigen peptides combined into up to 4 pools, with each peptide pool containing:
   Up to 5 peptides; each peptide at a concentration of 400m/mL
   4% dimethyl sulfoxide (DMSO) USP (United States Pharmacopeia) Grade
   4.9%-5.0% dextrose in water (D5W) injection
   ≤5.0 mM sodium succinate
Poly-ICLC—clinical grade poly I; poly C stabilized with carboxymethylcellulose and poly-L-lysine, which is composed of:
   1.8 mg/mL poly-inosinic acid: poly-cytidilic acid
   1.5 mg/mL poly-L-lysine
   5 mg/mL sodium carboxymethylcellulose
   0.9% sodium chloride

### Example 3: Generalized Method of Drug Preparation and Administration

Patients undergo a screening period to detect the type and prognosis of cancer. Enrolled patients undergo surgical or core needle biopsy of a primary or metastatic tumor site for DNA and RNA sequencing, immunological analysis, and, only for patients assigned to vaccine treatment, generation of neoantigenic vaccine. An archival biopsy sample that is obtained within 180 days of patient consent can be used only if the sample has been stored appropriately, meets tumor cellularity and quantity requirements, and if the patient has not received any inter-current therapy. Patients whose tumors do not have a sufficient number of mutations or epitopes to manufacture a vaccine are withdrawn from the study. In addition, each patient undergoes a blood draw to serve as a normal tissue reference for vaccine development and genotyping of HLA loci. Also, an 80-mL peripheral blood draw is completed for comprehensive immune monitoring.

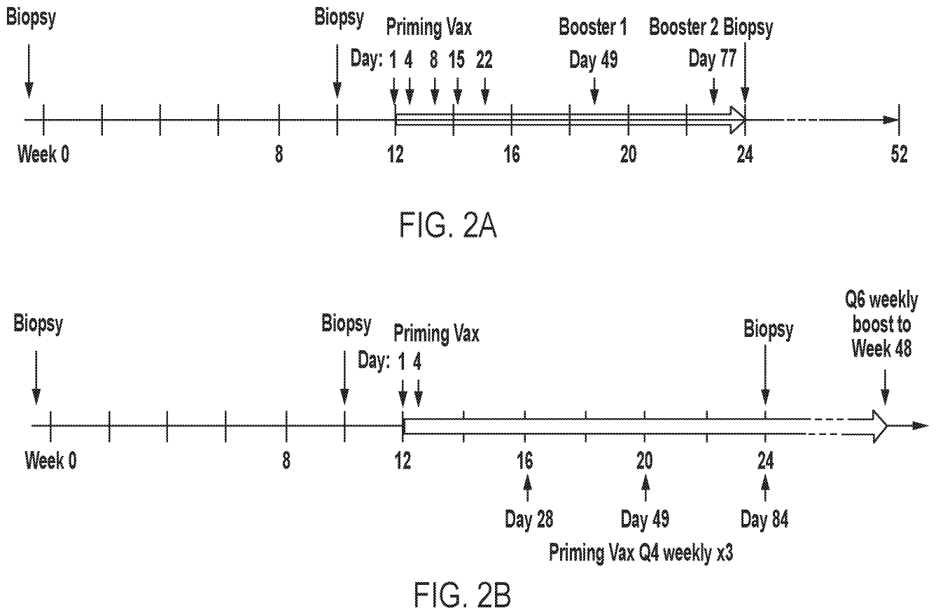

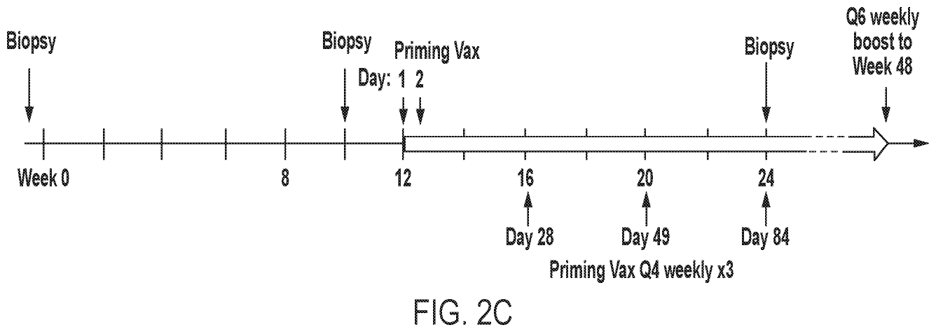

A treatment regimen comprises administration of multiple compositions such as the neoantigenic vaccine composition, ipilimumab, nivolumab, APX005M amongst others. If vaccination occurs on the same day as treatment with other study drug(s) described herein, then vaccination with neoantigenic composition can occur before administration of the other study drug(s). Exemplary schematic of a dosage regimen are described in FIG. 1 and FIG. 2A and FIG. 2B and FIG. 2C.

The pharmaceutical preparation is in unit dosage form. In such form the preparation is subdivided into unit doses containing appropriate quantities of the active component. The unit dosage form is a packaged preparation, the package containing discrete quantities of preparation, such as packeted tablets, capsules, and powders in vials or ampoules. Also, the unit dosage form can be a liquid, capsule, tablet, cachet, or lozenge itself, or it can be the appropriate number of any of these in packaged form.

In the instance where one or more compositions disclosed herein comprise a combination of a neoantigenic peptide and one or more additional therapeutic or prophylactic agents, such as a checkpoint inhibitor or a CD40 agonist, the one or more additional therapeutic or prophylactic agents, such as a checkpoint inhibitor or a CD40 agonist is administered at dosage levels of between about 1 to 100%, between about 5 to 95% of the dosage normally administered in a monotherapy regimen. In the instance where one or more compositions disclosed herein comprise a combination of a neoantigenic peptide and one or more additional therapeutic or prophylactic agents, such as a checkpoint inhibitor or a CD40 agonist, the neoantigenic peptide can be administered at dosage levels of between about 1 to 100%, between about 5 to 95% of the dosage normally administered in a monotherapy regimen. In the instance where one or more compositions disclosed herein comprise a combination of a neoantigenic peptide and one or more additional therapeutic or prophylactic agents, such as a checkpoint inhibitor or a CD40 agonist, the neoantigenic peptide and/or the one or more additional therapeutic or prophylactic agents, such as a checkpoint inhibitor or a CD40 agonist, can be administered at dosage levels of between about 1 to 100%, such as between about 5 to 95%, of the dosage normally administered in a monotherapy regimen.

In the instance where one or more compositions disclosed herein comprise a combination of a neoantigenic peptide and one or more additional therapeutic or prophylactic agents, such as a checkpoint inhibitor or a CD40 agonist, the neoantigenic peptide and/or the one or more additional therapeutic or prophylactic agents, such as a checkpoint inhibitor or a CD40 agonist, can be administered at dosage levels of greater than 1% and less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10% or less than 5% the dosage normally administered in a monotherapy regimen. In some embodiments, the neoantigenic peptide and one or more additional therapeutic or prophylactic agents, such as a checkpoint inhibitor or a CD40 agonist, are administered separately, as part of a multiple dose regimen. Alternatively, those one or more additional therapeutic or prophylactic agents, such as a checkpoint inhibitor or a CD40 agonist, can be a part of a single dosage form, mixed together with the neoantigenic peptides disclosed herein in a single composition.

In case of the one dosage regimens, administration of ipilimumab is in a prime boost regimen. In one dosage regimen, administration of ipilimumab is at vaccine day 1, 2, 3 or 4 as a prime. In one dosage regimen, administration of ipilimumab is at months 2 or 3 as a boost from the beginning of the vaccine treatment period. In some embodiments, in one dosage regimen, administration of ipilimumab is at day 49 as a boost from the beginning of the vaccination treatment period. In one dosage regimen, administration of APX005M is in a prime boost regimen. In one dosage regimen, administration of APX005M is at week 1, 2, 3, 4 or 5 as a prime. In one dosage regimen, administration of APX005M is at vaccine days 1 and 21 during the vaccine prime period. In some embodiments, administration of APX005M is at months 2 or 3 or vaccine day 49 as a boost from the beginning of the vaccine treatment period. In some embodiments, administration of APX005M is at months 2 or 3 as a boost from the beginning of the vaccination treatment period. In some embodiments, in one dosage regimen, administration of APX005M is at day 49 as a boost from the beginning of the vaccination treatment period.

In some aspects, provided herein is a method of treating or preventing metastatic melanoma in a human subject in need thereof that has been treated with nivolumab comprising administering to the subject: at least five peptides each comprising a unique neoepitope of a protein at a dose of from 200-400 μg of each peptide; and then an anti-CD40 agonist antibody that is APX005M at a dose of from 0.05-2.0 mg/kg.

In some aspects, provided herein is a method of treating or preventing metastatic melanoma in a human subject in need thereof that has been treated with nivolumab comprising administering to the subject: at least five peptides each comprising a unique neoepitope of a protein at a dose of from 200-400 μg of each peptide; and then ipilimumab at a dose of from 0.5-1.5 mg/kg.

In some aspects, provided herein is a method of treating or preventing cancer in a human subject in need thereof that has been treated with nivolumab comprising administering to the subject an anti-CD40 agonist antibody at a dose of less than 1.0 mg/kg or at a dose of less than 0.1 mg/kg. In some aspects, provided herein is a method of treating or preventing cancer in a human subject in need thereof that has been treated with nivolumab comprising administering to the subject an anti-CD40 agonist antibody at a dose of from 1-95% a dosage of the anti-CD40 agonist antibody normally administered in a monotherapy regimen. In some embodiments the anti-CD40 agonist antibody is APX005M. In some aspects, provided herein is a method of treating or preventing cancer in a human subject in need thereof that has been treated with nivolumab at a dose of from 1-95% a dosage of the nivolumab normally administered in a monotherapy regimen, the method comprising administering to the subject an anti-CD40 agonist antibody at a dose of less than 1.0 mg/kg or at a dose of less than 0.1 mg/kg. In some embodiments the anti-CD40 agonist antibody is APX005M. In some aspects, provided herein is a method of treating or preventing cancer in a human subject in need thereof that has been treated with nivolumab at a dose of from 1-95% a dosage of the nivolumab normally administered in a monotherapy regimen, the method comprising administering to the subject an anti-CD40 agonist antibody at a dose of from 1-95% a dosage of the anti-CD40 agonist antibody normally administered in a monotherapy regimen. In some embodiments the anti-CD40 agonist antibody is APX005M. In some aspects, provided herein is a method of treating or preventing cancer in a human subject in need thereof comprising administering to the subject: nivolumab at a dose of less than 1.0 mg/kg or at a dose of less than 3.0 mg/kg or at a dose of from 1-95% a dosage of the nivolumab normally administered in a monotherapy regimen; and an anti-CD40 agonist antibody at a dose of less than 1.0 mg/kg or at a dose of less than 0.1 mg/kg or at a dose of from 1-95% a dosage of the anti-CD40 agonist antibody normally administered in a monotherapy regimen. In some embodiments the anti-CD40 agonist antibody is APX005M. In some embodiments, the method further comprises administering to the subject at least five peptides each comprising a unique neoepitope of a protein at a dose of from 100-500 μg of each peptide.

In some aspects, provided herein is a method of treating or preventing cancer in a human subject in need thereof that has been treated with nivolumab comprising administering to the subject ipilimumab at a dose of less than 1.0 mg/kg. In some aspects, provided herein is a method of treating or preventing cancer in a human subject in need thereof that has been treated with nivolumab comprising administering to the subject ipilimumab at a dose of from 1-95% a dosage of the ipilimumab normally administered in a monotherapy regimen. In some aspects, provided herein is a method of treating or preventing cancer in a human subject in need thereof that has been treated with nivolumab at a dose of from 1-95% a dosage of the nivolumab normally administered in a monotherapy regimen, the method comprising administering to the subject ipilimumab at a dose of less than 1.0 mg/kg. In some aspects, provided herein is a method of treating or preventing cancer in a human subject in need thereof that has been treated with nivolumab at a dose of from 1-95% a dosage of the nivolumab normally administered in a monotherapy regimen, the method comprising administering to the subject ipilimumab at a dose of from 1-95% a dosage of the ipilimumab normally administered in a monotherapy regimen. In some aspects, provided herein is a method of treating or preventing cancer in a human subject in need thereof that has been treated with nivolumab comprising administering to the subject: nivolumab at a dose of less than 1.0 mg/kg or at a dose of less than 3.0 mg/kg or at a dose of from 1-95% a dosage of the nivolumab normally administered in a monotherapy regimen; and ipilimumab at a dose of less than 1.0 mg/kg or at a dose of from 1-95% a dosage of the ipilimumab normally administered in a monotherapy regimen. In some embodiments, the method further comprises administering to the subject at least five peptides each comprising a unique neoepitope of a protein at a dose of from 100-500 μg of each peptide.

Neoantigenic vaccine is administered subcutaneously starting at Week 12 of a study after the patient has been screened and completed a pre-treatment analysis. Patients can be vaccinated according to the cohort and treatment arm assignments described elsewhere herein. For patients whose first neoantigenic composition vaccination is administered earlier than Week 12 or delayed beyond Week 12, the timing of procedures that are linked to the vaccination schedule could be adjusted accordingly.

On the day of vaccination, 0.75 mL from one of each of the neoantigenic composition peptide pool vials can be separately mixed with 0.25 mL Poly-ICLC in a single syringe for injection. Each of the four neoantigenic composition vaccination syringes can be assigned to one of four extremities. At each vaccination, each neoantigenic composition vaccination syringe can be administered subcutaneously to the assigned extremity. Alternative anatomical locations for patients with complete axillary or inguinal lymph node dissection, or other contraindications that prevent injections to a particular extremity, can be the left and right midriff, respectively.

Nivolumab can be prepared according to the manufacturer's specifications. Patients can receive treatment with nivolumab according to the cohort and treatment arm assignments described elsewhere herein. The nivolumab infusion can be administered at a dose of 240 mg over 30 minutes through an IV line containing a sterile, non-pyrogenic, low-protein-binding in-line filter (pore size of 0.2 micrometer to 1.2 micrometer). Other drugs may not be co-administered through the same IV line. The IV line can be flushed at the end of the infusion. In some embodiments, nivolumab is administered at a dose of 240 mg every 2 weeks or 480 mg every 4 weeks. In some embodiments, nivolumab is administered at a dose of 1 mg/kg, followed by ipilimumab on the same day, every 3 weeks for 4 doses, then nivolumab at a dose of 240 mg every 2 weeks or 480 mg every 4 weeks.

When administration of all agents falls on the same day, the sequence of study drug administration can be neoantigenic composition, nivolumab, and, then, APX005M. The required volume of APX005M can be withdrawn from the vial and transferred into an IV container. APX005M may be diluted with 0.9% sodium chloride injection to prepare an infusion with a final concentration ranging from 1 mg/mL to 10 mg/mL. Concentrations as low as 0.04 mg/mL may be stable for up to 8 hours after preparation. The solution can be mixed by gentle inversion and may not be shaken.

The APX005M infusion may be administered at a dose of 0.1 mg/kg over 60 minutes through an IV line containing a sterile, non-pyrogenic, low-protein-binding in-line or add-on filter (pore size of 0.2 micron to 5 micron). Other drugs may not be co-administered through the same IV line. The IV line can be flushed at the end of the infusion. A window between −5 minutes and +10 minutes may be permitted (i.e., infusion time is 55 minutes to 70 minutes). The APX005M infusion can be interrupted in the case of infusion reaction. Once symptoms resolve, the infusion can be restarted at 50% of the initial infusion rate (e.g., from 50 mL/hr to 25 mL/hr).

The following guidance is provided regarding prevention and management of toxicities that might occur during APX005M administration: All patients can be premedicated approximately 30 minutes before the administration of the first dose of APX005M with a regimen containing: Oral H1 antagonist (e.g., loratadine 10 mg) or optional oral H2 antagonist (e.g., ranitidine 150 to 300 mg, cimetidine 300 to 800 mg, nizatidine 150 to 300 mg, and famotidine 20 to 40 mg) or oral nonsteroidal anti-inflammatory drug (may comprise ibuprofen 400 mg or equivalent) or Acetaminophen 650 mg.

In the cases where the time between premedication and the scheduled APX005M administration exceeds 4 hours, or if a patient experiences a grade 2 infusion reaction to nivolumab or any other study drug, patients may receive an additional course of premedication prior to APX005M administration.

The following guidance is provided regarding prevention and management of toxicities that might occur after APX005M administration: ensure that patients are well-hydrated prior to discharge; consider administration of IV fluids; instruct the patient to drink volume-increasing fluids (e.g., Gatorade, broth) for the remainder of the infusion day and maintain an adequate oral fluid intake for the first 24-48 hours after APX005M administration. In some cases, prophylactic fever management for the first 24 hours (e.g., ibuprofen 400-600 mg every 8 hours, alternating with acetaminophen 1000 mg every 6 hours) may be considered. In some cases, withholding anti-hypertensive medications on the day of APX005M administration may be considered if such action poses no risk to the patient.

When administration of all agents falls on the same day, the sequence of study drug administration can be neoantigenic composition, nivolumab, and, then, ipilimumab. Ipilimumab can be prepared according to the manufacturer's specifications. Patients can receive treatment with ipilimumab according to the cohort and treatment arm assignments described elsewhere herein.

The ipilimumab infusion can be administered at a dose of 1.0 mg/kg over 90 minutes through an IV line containing a sterile, nonpyrogenic, low-protein-binding in-line filter. Ipilimumab may not be mixed with or administered as an infusion with other medicinal products. The IV line can be flushed at the end of the infusion.

### Example 4: Treatment Regimen for Patients Using Nivolumab and Neoantigens

In this example, treatment of patients with melanoma with a combination of an immune checkpoint modulator such as nivolumab and the neoantigen peptides is described. The combination of immunogenic neoantigenic peptides and nivolumab is expected to have better results than treatment performed separately. The vaccine composition of neoantigenic peptides is mixed with an adjuvant such as poly-ICLC for reasons mentioned above.

The vaccine composition consists of up to 20 neoantigen peptides of approximately 14 to 35 amino acids in length, derived from sequence data from neoantigens in each patient's tumor. Personalized, synthesized neoantigen peptides are be split into 4 pools with up to 5 peptides each.

The vaccine composition is administered in a prime-boost schedule over approximately 12 weeks in Treatment Arms A and B as 5 priming vaccinations followed by 2 booster vaccinations as shown in FIG. 2A, Table 1. In Cohort 1 Treatment Arm C, an alternative vaccination schedule is followed over approximately 36 or 48 weeks with the vaccine composition administered as 5 priming vaccinations followed by 4 booster vaccinations as shown in FIG. 2B, Table 2. For each vaccination time point in Treatment Arms A and B, the vaccine composition is administered at a volume of 1 mL by subcutaneous injection at 4 peripheral sites (1 peptide pool per site). The vaccine composition can be administered to the patients in arm A on days 1, 4, 8, 15 and 22 and booster doses are provided on days 49 and 77 from the beginning of the vaccine treatment period. The vaccine composition is administered to the patients in arm C on Day 1, 2, 28, 49, 84 and booster doses can be provided on days 126, 168, 210 and 252 from the beginning of the vaccine treatment period.

All patients receive 240 mg of nivolumab, administered by IV infusion every 2 weeks from Day 1/Week 0 to the end of the Post-Vaccination Treatment Period. The nivolumab administration is continued in treatment arms A and B for up to 12 weeks and up to 36 weeks in treatment arm C. As a control, patients in Cohort 1 Treatment Arm B receive adjuvant (poly-ICLC) (Table 1).

Assessment of the patient's health is performed with each administration of the combination therapy. Tests that are performed have been described elsewhere herein. Patients with other types of neoplasia may also benefit from such a treatment regimen with some alterations. All assessments may occur during one of these phases.

An exemplary schedule of treatment using Nivolumab and neoantigens with the assessments to be performed is provided in Table 1 and FIG. 1, FIG. 2A, FIG. 2B, and FIG. 2C.

TABLE 1
Treatment and Assessment Schedule for Nivolumab and Neoantigens

| | Vaccination Treatment Period¹ | | | | | | | | | | | |
|---|---|---|---|---|---|---|---|---|---|---|---|---|
| | Study Week | | | | | | | | | | | |
| | Wk 12 | Wk 12 | | | | | | | | | | |
| | Day 1 | Day 4 | Wk 13 | Wk 14 | Wk 15 | Wk 16 | Wk 18 | Wk 19 | Wk 20 | Wk 22 | Wk 23 | Wk 24² |
| | Days Relative to First Vaccine: | | | | | | | | | | | |
| | 1 | 4 | 8 | 15 | 22 | 29 | 43 | 50 | 57 | 71 | 78 | 85 |
| Treatment | (±1 d) | (±1 d) | (±3 d) | (±2 d) | (±3 d) | (±2 d) | (±2 d) | (±7 d) | (±2 d) | (±2 d) | (±7 d) | (±2 d) |
| Nivolumab | Nivolumab administered at 240 mg IV Q2W to Week 52. Nivolumab administration window is | | | | | | | | | | | |
| | Q2W ±2 days, with a minimum of 12 days between infusions. | | | | | | | | | | | |
| Arm A: | X | X | X | X | X | | | X | | | X | |
| Neoantigen vaccination¹ | Prime 1 | Prime 2 | Prime 3 | Prime 4 | Prime 5 | | | Boost 1 | | | Boost 2 | |
| Arm B: | X | X | X | X | X | | | X | | | X | |
| Poly-ICLC | | | | | | | | | | | | |
| administration¹ | | | | | | | | | | | | |
| Procedure/Assessment¹ | | | | | | | | | | | | |
| Symptom-directed | X | X | X | X | X | X | X | X | X | X | X | X |
| physical examination³ | | | | | | | | | | | | |
| ECOG PS | X | | | | | | | | | | | X |
| Vital signs | X | X | X | X | X | X | X | X | X | X | | X |
| CT/MRI^{1, 4} | X | | | | | | | | | | | X |
| Hematology⁵ | X | | | | | X | | | X | | | X |
| Chemistry⁵ | X | | | | | X | | | X | | | X |
| Pregnancy testing⁶ | X | | | | | X | | | X | | | X |
| Leukapheresis^{1, 7} | | | | | | | | | X | | | |
| Tumor biopsy^{9, 10} | | | | | | | | | | | | X |
| 80-mL blood draw for | | | | X | | X | | | | | | X |
| immune monitoring¹ | | | | | | | | | | | | |

| | |
|---|---|
| AE and SAE collection | SAEs will be collected from signing of ICF throughout study; AEs from Day 1/Week 0 through the |
| | 30-day Post-treatment Period visit |
| Concomitant medications | Collected front 30 days prior to Day 1/Week 0 throughout study |
| and procedures | |

Abbreviations: AE = adverse event; ; β-hCG = Beta-human chorionic gonadotropin; CT = computed tomography; d = days; ECG = electrocardiogram; ECOG PS = Eastern Cooperative Oncology Group Performance Score; HLA = human leukocyte antigen; ICF = informed consent form; IV = intravenous; MRI = magnetic resonance imaging; OS = overall survival; PD = progressive disease; Q2W = every 2 weeks; RECIST = Response Evaluation Criteria in Solid Tumors; SAE = serious adverse event; Tx = treatment; Wk = week
¹At the discretion of the Investigator, patients with PD may begin the Vaccination Treatment Period earlier than 12 weeks, if NEO-PV-01 vaccine is available, while continuing therapy with nivolumab. For these patients, the timing of all treatments and assessments should be adjusted accordingly.
²After assessments at Week 24 of the Vaccination Treatment Period, refer to Table 5 for assessments during the post-Vaccination Treatment Period.
³A symptom-directed physical exam must be conducted at all study visits.
⁴CT or MRI of other areas of disease (e.g., neck) should be obtained as clinically indicated. If IV contrast is contraindicated, a non-contrast CT and MRI may be used to evaluate sites of disease where a CT without contrast is not adequate. The imaging modality should remain consistent throughout the study and should be the same modality used at Screening. Clinical assessments must be based on RECIST v1.1.
⁵Collect all samples prior to dosing.
⁶Women of childbearing potential must have a negative urine or serum β-hCG or urine pregnancy test during Screening (within 7 days of starting treatment). Urine pregnancy testing will be performed at subsequent study visits.
⁷Patients will undergo a leukapheresis procedure to collect peripheral blood mononuclear cells for comprehensive immune monitoring. If leukapheresis cannot be performed for safety reasons, a total of 180 mL to 200 mL of blood should be drawn as a replacement for this procedure.
8. The Week 20 leukapheresis may be completed within a 15-day window following the study visit and must occur after the first booster vaccination (Week 19). The procedure must be completed prior to any scheduled treatment that day.
⁹Additional tumor biopsy to be obtained in patients who show PD and are discontinued from the study.
¹⁰The Week 24 biopsies may be completed within a 15-day window following the study visit and must occur after the final vaccination (Week 24).

TABLE 2
Treatment and Assessment Schedule for Nivolumab and Neoantigens Alternative Vaccination Schedule

| | Vaccination Treatment Period¹ | | | | | | | | | | | | |
|---|---|---|---|---|---|---|---|---|---|---|---|---|---|
| | Study Week | | | | | | | | | | | | |
| | Wk 12 | Wk 12 | | | | | | | | | | | |
| | Day 1 | Day 2 | Wk 14 | Wk 16 | Wk 18 | Wk 19 | Wk 20 | Wk 22 | Wk 24 | Wk 30 | Wk 36 | Wk 42 | Wk 48² |
| | Days Relative to First Vaccine: | | | | | | | | | | | | |
| | 1 | 2 | 15 | 29 | 43 | 50 | 57 | 71 | 85 | 127 | 169 | 211 | 253 |
| Treatment | (±1 d) | (±1 d) | (±2 d) | (±2 d) | (±2 d) | (±7 d) | (±2 d) | (±2 d) | (±2 d) | (±2 d) | (±2 d) | (±2 d) | (±2 d) |
| Nivolumab | Nivolumab administered at 240 mg IV Q2W to Wk 60. Nivolumab administration window is | | | | | | | | | | | | |
| | Q2W ±2 days, with a minimum of 12 days between infusions. | | | | | | | | | | | | |
| Arm C: | X | X | | X | | X | | | X | X | X | X | X |
| (alternative schedule) | Prime 1 | Prime 2 | | Prime 3 | | Prime 4 | | | Prime 5 | Boost 1 | Boost 2 | Boost 3 | Boost 4 |
| Neoantigen vaccination¹ | | | | | | | | | | | | | |
| Procedure/Assessment¹ | | | | | | | | | | | | | |
| Symptom-directed | X | X | X | X | X | X | X | X | X | X | X | X | X |
| physical | | | | | | | | | | | | | |
| examination³ | | | | | | | | | | | | | |
| ECOG PS | X | | | | | | | | X | | X | | X |
| Vital signs | X | X | X | X | X | | X | X | X | X | X | X | X |
| CT/MRI^{1, 4} | X | | | | | | | | X | | | | X |
| Hematology⁵ | X | | | X | | | X | | X | X | X | X | X |
| Chemistry⁵ | X | | | X | | | X | | X | X | X | X | X |
| Pregnancy testing⁶ | X | | | X | | | X | | X | X | X | X | X |
| Leukapheresis^{1, 7} | | | | | | | | | X⁹ | | | | |
| Tumor biopsy¹⁰ | | | | | | | | | X | | | | |
| 80-mL blood draw for | | | | X⁸ | | | | | | X⁸ | X⁸ | X⁸ | |
| immune monitoring¹ | | | | | | | | | | | | | |

| | |
|---|---|
| AE and SAE collection | SAEs will be collected from signing of ICF throughout study; AEs from Day 1/Week 0 through the |
| | 30-day Post-treatment Period Visit |
| Concomitant medications | Collected from 30 days prior to Day 1/Week 0 throughout study |
| and procedures | |

Abbreviations: AE = adverse event; β-hCG = Beta-human chorionic gonadotropin; CT = computed tomography; d = days; ECG = electrocardiogram; ECOG PS = Eastern Cooperative Oncology Group Performance Score; HLA = human leukocyte antigen; ICF = informed consent form; IV = intravenous; MRI = magnetic resonance imaging; OS = overall survival; PD = progressive disease; Q2W = every 2 weeks; RECIST = Response Evaluation Criteria in Solid Tumors; SAE = serious adverse event; Tx = treatment; Wk = week
¹At the discretion of the Investigator, patients with PD may begin the Vaccination Treatment Period earlier titan 12 weeks, if NEO-PV-01 vaccine is available, while continuing therapy with nivolumab. For these patients, the timing of all treatments and assessments should be adjusted accordingly, where applicable.
²After assessments at Week 48 of the Vaccination Treatment Period, refer to Table 5 for assessments during the Post- Vaccination Treatment Period.
³A symptom-directed physical exam must be conducted at all study visits.
⁴CT or MRI of other areas of disease (e.g., neck) should be obtained as clinically indicated. If IV contrast is contraindicated, a non-contrast CT and MRI may be used to evaluate sites of disease where a CT without contrast is not adequate. The imaging modality should remain consistent throughout the study and should be the same modality used at Screening. Clinical assessments must be based on RECIST v1.1.
⁵Collect all samples prior to dosing.
⁶Women of childbearing potential must have a negative urine or serum β-hCG pregnancy test during Screening (within 7 days of starting treatment). Urine pregnancy testing will be performed at subsequent study visits.
⁷Patients will undergo a leukapheresis procedure to collect peripheral blood mononuclear cells for comprehensive immune monitoring. A 15-day window prior to the study visit is allowed for leukapheresis, but the procedure must be completed prior to any scheduled treatment. If leukapheresis cannot be performed for safety reasons, a total of 180 mL to 200 mL of blood should be drawn as a replacement for this procedure.
⁸The Week 24 leukapheresis may be completed within a 15-day window following the study visit and must occur after the final priming vaccination (Week 24). The procedure must be completed prior to any scheduled treatment that day.
⁹Conduct assessment 1 week after vaccination.
¹⁰Additional tumor biopsy to be obtained in patients who show PD and are discontinued from the study.
11. The Week 24 biopsies may be completed within a 15-day window following the study visit and must occur after the final priming vaccination (Week 24).

### Example 5: Treatment Regimen for Patients Using Nivolumab, Neoantigens and APX005M

In this example, a treatment regimen of patients with a metastatic disease such as melanoma is described. The treatment encompasses administration of a combination of an immune checkpoint modulator such as nivolumab, an anti-CD40 antibody such as APX005M and the neoantigen peptides as described herein. The combination of immunogenic neoantigenic peptides, APX005M and nivolumab is expected to have better results than treatment performed separately. The vaccine composition of neoantigenic peptides can be mixed with an adjuvant such as poly-ICLC for reasons mentioned above.

The vaccine composition consists of up to 20 neoantigen peptides of approximately 14 to 35 amino acids in length, derived from sequence data from neoantigens in each patient's tumor. Personalized, synthesized neoantigen peptides are be split into 4 pools with up to 5 peptides each.

Patients in treatment arms A and B receive 0.1 mg/kg of APX005M administered as an intravenous (IV) infusion over 60 minutes at Week 12, Week 15, and Week 19 of the vaccination treatment period. The vaccine composition is administered in a prime-boost schedule over approximately 12 weeks in Treatment Arm A as 5 priming vaccinations followed by 2 booster vaccinations. The vaccine composition is administered to the patients in arm A on days 1, 4, 7, 14, 21 and booster doses can be provided on days 49 and 77 from the beginning of the treatment period.

All patients receive 240 mg of nivolumab, administered by IV infusion every 2 weeks from Day 1/Week 0 to the end of the Post-Vaccination Treatment Period. The nivolumab administration is continued in treatment arms A and B for up to 12 weeks. As a control, Arm B can be administered only a combination of APX005M and nivolumab.

Assessment of the patient's health is performed with each administration of the combination therapy. Tests that are performed have been described elsewhere herein. Patients with other types of neoplasia may also benefit from such a treatment regimen with some alterations.

An exemplary schedule of treatment using Nivolumab, APX005M and Neoantigens with the assessments to be performed is provided in Table 3.

TABLE 3
Treatment and Assessment Schedule for Nivolumab, APX005M and Neoantigens

| | Vaccination Treatment Period¹ | | | | | | | | | | | | |
|---|---|---|---|---|---|---|---|---|---|---|---|---|---|
| | Study Week | | | | | | | | | | | | |
| | Wk 12 | Wk 12 | Wk 12 | | | | | | | | | | |
| | Day 1 | Day 2 | Day 4 | Wk 13 | Wk 14 | Wk 15 | Wk 16 | Wk 18 | Wk 19 | Wk 20 | Wk 22 | Wk 23 | Wk 24² |
| | Days Relative to First Vaccine: | | | | | | | | | | | | |
| | 1 | | 4 | 8 | 15 | 22 | 29 | 43 | 50 | 57 | 71 | 78 | 85 |
| Treatment | (±1 d) | 2 | (±1 d) | (±2 d) | (±2 d) | (±2 d) | (±2d) | (±2 d) | (±7 d) | (±2 d) | (±2 d) | (±7 d) | (±2 d) |
| Nivolumab | Nivolumab administered at 240 mg IV Q2W to Week 52. Nivolumab administration window is | | | | | | | | | | | | |
| | Q2W ±2 days, with a minimum of 12 days between infusions. | | | | | | | | | | | | |
| Arm A: | X | | X | X | X | X | | | X | | | X | |
| Neoantigen vaccination¹ | Prime 1 | | Prime 2 | Prime 3 | Prime 4 | Prime 5 | | | Boost 1 | | | Boost 2 | |
| Arm A: | X | | | | | X | | | X | | | | |
| APX005M | | | | | | | | | | | | | |
| (0.1 mg/kg IV) | | | | | | | | | | | | | |
| administration¹ | | | | | | | | | | | | | |
| Arm B: | X | | | | | X | | | X | | | | |
| APX005M | | | | | | | | | | | | | |
| (0.1 mg/kg IV) | | | | | | | | | | | | | |
| administration¹ | | | | | | | | | | | | | |
| Procedure/Assessment¹ | | | | | | | | | | | | | |
| Symptom-directed | X | X | X⁴ | X⁴ | X | X | X | X | X | X | X | X | X |
| physical examination³ | | | | | | | | | | | | | |
| ECOG PS | X | | | | | | | | | | | | X |
| Vital signs | X | X | X⁴ | X⁴ | X | X | X | X | X | X | X | X | X |
| CT/MRI^{1, 5} | X | | | | | | | | | | | | X |
| Hematology⁶ | X | | | | | | X | | | X | | | X |
| Chemistry⁶ | X | | | | | | X | | | X | | | X |
| Pregnancy testing | X | | | | | | X | | | X | | | X |
| Thyroid-stimulating | X | | | | | X | | | X | | | | |
| hormone^{6, 8} | | | | | | | | | | | | | |
| Leukapheresis^{1, 9, 10} | | | | | | | | | | X | | | |
| Tumor biopsy^{11, 12} | | | | | | | | | | | | | X |
| 80-mL blood draw for | | | | | X | | X | | | | | | X |
| immune monitoring¹ | | | | | | | | | | | | | |

| | |
|---|---|
| AE and SAE collection | SAEs will be collected from signing of ICF throughout study; AEs from Day 1/Week 0 through the |
| | 30-day Post-treatment Period Visit |
| Concomitant medications | Collected from 30 days prior to Day 1/Week 0 throughout study |
| and procedures | |

Abbreviations: AE = adverse event; β-hCG = Beta-human chorionic gonadotropin; CT = computed tomography; d = days; ECG = electrocardiogram; ECOG PS = Eastern Cooperative Oncology Group Performance Score; HLA = human leukocyte antigen; ICF = informed consent form; IV = intravenous; MRI = magnetic resonance imaging; OS = overall survival; PD = progressive disease; Q2W = every 2 weeks; RECIST = Response Evaluation Criteria in Solid Tumors; SAE = serious adverse event; Tx = treatment; Wk = week
¹At the discretion of the Investigator, patients with PD may begin the Vaccination Treatment Period earlier than 12 weeks, if NEO-PV-01 vaccine is available, while continuing therapy with nivolumab. For these patients, the timing of all treatments and assessments should be adjusted accordingly.
²After assessments at Week 24 of the Vaccination Treatment Period, refer to Table 5 for assessments during the Post-Vaccination Treatment Period.
³A symptom-directed physical exam must be conducted at all study visits.
⁴Patients treated in Arm B do not have a visit at this time point.
⁵CT or MRI of other areas of disease (e.g., neck) should be obtained as clinically indicated. If IV contrast is contraindicated, a non-contrast CT and MRI may be used to evaluate sites of disease where a CT without contrast is not adequate. The imaging modality should remain consistent throughout the study and should be the same modality used at Screening. Clinical assessments must be based on RECIST v1.1.
⁶Collect all samples prior to dosing.
7. Women of childbearing potential must have a negative urine or serum β-hCG pregnancy test during Screening (within 7 days of starting treatment). Urine pregnancy testing will be performed at subsequent study visits.
⁸Thyroid-stimulating hormone with reflex T4.
⁹Patients will undergo a leukapheresis procedure to collect peripheral blood mononuclear cells for comprehensive immune monitoring. A 15-day window prior to the study visit is allowed for leukapheresis. but the procedure must be completed prior to any scheduled treatment. If leukapheresis cannot be performed for safety reasons, a total of 180 mL to 200 mL of blood should be drawn as a replacement for this procedure.
¹⁰The Week 20 leukapheresis may be completed within a 15-day window following the study visit and must occur after the first booster vaccination (Week 19). The procedure must be completed prior to any scheduled treatment that day.
¹¹Additional tumor biopsy to be obtained in patients who show PD and are discontinued from the study.
¹²The Week 24 biopsies may be completed within a 15-day window following the study visit and must occur after the final vaccination (Week 24).

### Example 6: Treatment Regimen for Patients Using Nivolumab, Neoantigens and Ipilimumab

In this example, treatment of patients with a metastatic disease such as melanoma with a combination of an immune checkpoint modulator such as nivolumab, an anti-CTLA4 antibody such as ipilimumab and the neoantigen peptides is described. The combination of immunogenic neoantigenic peptides, ipilimumab and nivolumab is expected to have better results than treatment performed separately. The vaccine composition of neoantigenic peptides can be mixed with an adjuvant such as poly-ICLC for reasons mentioned above.

The vaccine composition consists of up to 20 neoantigen peptides of approximately 14 to 35 amino acids in length, derived from sequence data from neoantigens in each patient's tumor. Personalized, synthesized neoantigen peptides can then be split into 4 pools with up to 5 peptides each.

Patients in treatment arms A and B receive 1.0 mg/kg of ipilimumab administered as an IV infusion over 90 minutes at Week 12 and Week 19 of the vaccination treatment period. The vaccine composition is administered in a prime-boost schedule over approximately 12 weeks in Treatment Arm A as 5 priming vaccinations followed by 2 booster vaccinations. The vaccine composition is administered to the patients in arm A on days 1, 4, 7, 15, 21 and booster doses are provided on days 49 and 77 from the beginning of the treatment period.

All patients receive 240 mg of nivolumab, administered by IV infusion every 2 weeks from Day 1/Week 0 to the end of the Post-Vaccination Treatment Period. The nivolumab administration is continued in treatment arms A and B for up to 12 weeks. As a control, Arm B is administered only a combination of ipilimumab and nivolumab.

Assessment of the patient's health is performed with each administration of the combination therapy. Tests that are performed have been described elsewhere herein. Patients with other types of neoplasia may also benefit from such a treatment regimen with some alterations.

An exemplary schedule of treatment using Nivolumab, Ipilimumab and Neoantigens with the assessments to be performed is provided in Table 4.

TABLE 4
Treatment and Assessment Schedule for Nivolumab, Ipilimumab and Neoantigens

| | Vaccination Treatment Period¹ | | | | | | | | | | | |
|---|---|---|---|---|---|---|---|---|---|---|---|---|
| | Study Week | | | | | | | | | | | |
| | Wk 12 | Wk 12 | | | | | | | | | | |
| | Day 1 | Day 4 | Wk13 | Wk 14 | Wk 15 | Wk16 | Wk 18 | Wk 19 | Wk 20 | Wk 22 | Wk 23 | Wk 24² |
| | Days Relative to First Vaccine: | | | | | | | | | | | |
| | 1 | 4 | 8 | 15 | 22 | 29 | 43 | 50 | 57 | 71 | 78 | 85 |
| Treatment | (±1 d) | (±1 d) | (±3 d) | (±2 d) | (±3 d) | (±2 d) | (±2 d) | (±7 d) | (±2 d) | (±2 d) | (±7 d) | (±2 d) |
| Nivolumab | Nivolumab administered at 240 mg IV Q2W to Week 52. Nivolumab administration window is | | | | | | | | | | | |
| | Q2W ±2 days, with a minimum of 12 days between infusions. | | | | | | | | | | | |
| Arm A: | X | X | X | X | X | | | X | | | X | |
| Neoantigen vaccination¹ | Prime 1 | Prime 2 | Prime 3 | Prime 4 | Prime 5 | | | Boost 1 | | | Boost 2 | |
| Arm A: | X | | | | | | | X | | | | |
| Ipilimumab (1.0 mg/kg IV) | | | | | | | | | | | | |
| administration¹ | | | | | | | | | | | | |
| Arm B: | X | | | | | | | X | | | | |
| Ipilimumab (1.0 mg/kg IV) | | | | | | | | | | | | |
| administration¹ | | | | | | | | | | | | |
| Procedure/Assessment¹ | | | | | | | | | | | | |
| Symptom-directed physical | X | X⁴ | X⁴ | X | X⁴ | X | X | X | X | X | X | X |
| examination³ | | | | | | | | | | | | |
| ECOG PS | X | | | | | | | | | | | X |
| Vital signs | X | X⁴ | X⁴ | X | X⁴ | X | X | X | X | X | | X |
| CT/MRI^{1, 5} | X | | | | | | | | | | | X |
| Hematology⁶ | X | | | | | X | | | X | | | X |
| Chemistry⁶ | X | | | | | X | | X | X | | | X |
| Pregnancy testing⁷ | X | | | | | X | | | X | | | X |
| Thyroid-stimulating | X | | | | | | | X | | | | X |
| hormone^{6, 8} | | | | | | | | | | | | |
| Leukapheresis^{1, 9, 10} | | | | | | | | | X | | | |
| Tumor biopsy^{11, 12} | | | | | | | | | | | | X |
| 80-mL blood draw for | | | | X | | X | | | | | | X |
| immune monitoring¹ | | | | | | | | | | | | |

| | |
|---|---|
| AE and SAE collection | SAEs will be collected from signing of ICF throughout study; AEs from Day 1/Week 0 through the |
| | 30-day Post-treatment Period Visit |
| Concomitant medications | Collected from 30 days prior to Day 1/Week 0 throughout study |
| and procedures | |

Abbreviations: AE = adverse event; β-hCG = Beta-human chorionic gonadotropin; CT = computed tomography; d = days; ECG = electrocardiogram; ECOG PS = Eastern Cooperative Oncology Group Performance Score; HLA = human leukocyte antigen; ICF = informed consent form; IV = intravenous; MRI = magnetic resonance imaging; OS = overall survival; PD = progressive disease; Q2W = every 2 weeks; RECIST = Response Evaluation Criteria in Solid Tumors; SAE = serious adverse event; Tx = treatment; Wk = week
¹At the discretion of the Investigator, patients with PD may begin the Vaccination Treatment Period earlier than 12 weeks, if NEO-PV-01 vaccine is available.
²After assessments at Week 24 of the Vaccination Treatment Period, refer to Table 5 for assessments during the Post-Vaccination Treatment Period.
³A symptom-directed physical exam must be conducted at all study visits.
⁴Patients treated in Arm B do not have a visit at this time point.
⁵CT or MRI of other areas of disease (e.g., neck) should be obtained as clinically indicated. If IV contrast is contraindicated, a non-contrast CT and MRI may be used to evaluate sites of disease where a CT without contrast is not adequate. The imaging modality should remain consistent throughout the study and should be the same modality used at Screening. Clinical assessments must be based on RECIST v1.1.
⁶Collect all samples prior to dosing.
⁷Women of childbearing potential must have a negative urine or serum β-hCG pregnancy test during Screening (within 7 days of starting treatment). Urine pregnancy testing will be performed at subsequent study visits.
⁸Thyroid-stimulating hormone with reflex T4.
⁹Patients will undergo a leukapheresis procedure to collect peripheral blood mononuclear cells for comprehensive immune monitoring. A 15-day window prior to the study visit is allowed for leukapheresis, but the procedure must be completed prior to any scheduled treatment. If leukapheresis cannot be performed for safety reasons, a total of 180 mL to 200 mL of blood should be drawn as a replacement for this procedure.
¹⁰The Week 20 leukapheresis may be completed within a 15-day window following the study visit and must occur after the first booster vaccination (Week 19). The procedure must be completed prior to any scheduled treatment that day.
¹¹Additional tumor biopsy to be obtained in patients who show PD and are discontinued from the study.
¹²The Week 24 biopsies may be completed within a 15-day window following the study visit and must occur after the final vaccination (Week 24).

### Example 7: Drug Dose Modification, Dose Delays and Discontinuation of Treatment with APX005M

In cases where patients experience a toxicity that requires a dose hold for nivolumab or APX005M or ipilimumab then the treatment is stopped. If nivolumab, APX005M, or ipilimumab is held for any reason, then vaccination with neoantigens may be held until the other drug is resumed.

Treatment with neoantigenic vaccine can be held for any of the following: a) Grade 3 or higher injection site reaction; b) Grade 3 or higher myalgias not adequately managed by antipyretics or c) Grade 3 or higher fevers not adequately managed by antipyretic treatment. Vaccine administration may be resumed in patients when AEs resolve to Grade 1 or less. If Grade 2 or greater symptoms last for more than 7 days during priming vaccination, any further doses may be stopped till a recourse is found.

If APX005M administration is held, then nivolumab can also be held until APX005M is resumed. Vaccination with neoantigens can be held until nivolumab is resumed. There will be no intra-patient dose modifications for nivolumab. Nivolumab doses may or may not be reduced.

Patients who require delay of nivolumab should be re-evaluated weekly or more frequently if clinically indicated and resume nivolumab dosing when retreatment criteria are met. Immune-mediated adverse events (IMAEs) are defined as serious or non-serious AEs consistent with an immune-mediated mechanism or immune-mediated component for which non-inflammatory etiologies (such as infection or tumor progression) have been ruled out. These IMAEs can include events with an alternate etiology which were exacerbated by the induction of autoimmunity. Management of IMAEs should follow the manufacturer's guidance provided in the Opdivo product labeling (Opdivo Package Insert, 2018).

Suspected adverse reactions (SARs) associated with APX005M exposure may represent an immunologic etiology. These adverse events may occur shortly after the administration of investigational products or several months after the last dose of investigational product. Management of SARs may require treatment hold, dose reduction, or discontinuation of investigational product(s) as shown in Table 1. If a patient experiences several toxicities, the recommended dose modification should be based on the highest grade toxicity.

### Example 8: Prior and Concomitant Medications

All medications taken within 30 days prior to the first dose of study treatment may be recorded in the electronic case report form (eCRF). In addition, all prior antineoplastic treatments for the underlying metastatic disease can be recorded in the eCRF.

Medications and treatments other than those specified elsewhere herein may be permitted during the study or the treatment course. All intercurrent medical conditions and complications of the underlying metastatic disease may be treated at the discretion of the care team according to acceptable local standards of medical care. Patients can receive analgesics, antiemetics, anti-infectives, antipyretics, and blood products as necessary.

All treatments that the care team considers necessary for a patient's welfare may be administered at the discretion of the Investigator in keeping with the community standards of medical care. All concomitant medication may be recorded on the eCRF including all prescription, over-the-counter, herbal supplements, and IV medications and fluids. If changes occur during the treatment Phase, documentation of drug dosage, frequency, route, and date may also be included on the eCRF.

Patients should inform the care team of any new medication or significant non-drug therapies (i.e., blood transfusions) including vitamins, supplements, etc., administered after the start of study treatment.

Patients may be prohibited from receiving some therapies during the screening and treatment phase. Examples of such therapies include: antineoplastic systemic chemotherapy or biological therapy; Immunotherapy other than the treatment regimen; investigational agents other than neoantigens, Poly-ICLC, APX005M, and ipilimumab; radiation therapy; non-oncology vaccine therapy; annual influenza vaccinations (for up to 8 weeks after the last neoantigen booster shot); systemic corticosteroids (>10 mg daily prednisone equivalents) or other immunosuppressive medications with the exception of steroids for the treatment of nivolumab-related immune AEs; premedication with steroids; prednisone-based: 50 mg prednisone by mouth at 13 hours, 7 hours, and 1 hour before contrast medium administration plus 50 mg diphenhydramine intravenously, intramuscularly, or by mouth 1 hour before contrast medium administration or methylprednisolone-based: 32 mg methylprednisolone by mouth 12 hours and 2 hours before contrast medium administration plus 50 mg diphenhydramine intravenously, intramuscularly, or by mouth 1 hour before contrast medium administration.

Patients taking narrow therapeutic index medications (such as warfarin, phenytoin, quinidine, carbamazepine, phenobarbital, cyclosporine, and digoxin) can be monitored proactively.

Pre-vaccination Period Assessments may include:
Adjust the timing of these assessments so that they are performed within 15 days prior to the first neoantigen priming vaccine dose:
   Imaging (CT/MRI)
   Tumor biopsy
   Vaccination Period Assessments
Adjust the timing of these assessments relative to the vaccine timing:
   80-mL blood draw (immune monitoring)
   Leukopheresis
   Post-Vaccination
Adjust the timing of these assessments so that they are performed 15 days after the final neoantigen booster vaccine dose:
   Tumor biopsy
   Imaging (CT/MRI)
   80-mL blood draw (immune monitoring)

### Example 9: Patient Criteria

Patients who may benefit from treatment with the above mentioned regimes include but are not limited to patients with advanced or metastatic melanoma. Patients who have previously not been administered therapies for the metastatic disease are included. Also included are patients who have an anticipated life expectancy of more than 6 months. Patients who may most benefit from the treatment include patients with White blood cell (WBC) count ≥3×10³/μL; Platelet count ≥100×10³/μL; Hemoglobin >9 g/dL; Serum creatinine ≤1.5× upper limit of normal (ULN); Aspartate aminotransferase (AST) and alanine aminotransferase (ALT)≤2.5×ULN or ≤5×ULN for patients with liver metastases; Total bilirubin ≤1.5×ULN (except in patients with Gilbert Syndrome who can have total bilirubin <3.0 mg/dL). Depending on the patient and their type of cancer, female patients who are pregnant or breastfeeding are not enrolled for treatment.

In some cases, patients who react adversely to treatment may be removed from a treatment regimen. Patients showing a grade 3 or higher injection site reaction, grade 3 or higher myalgias not adequately managed by antipyretics and grade 3 or higher fevers not adequately managed by antipyretic treatment are excluded from treatment in some cases.

Depending on the patient's health status, the type of neoplasia, age and weight they may or may not benefit significantly from treatment with the compositions provided herein. Patients who have been treated previously with compositions comprising anti-PD-1, anti-PD-L1, anti-CD40, or anti-CTLA-4 antibody therapies may not benefit from the combination therapy including such antibodies and neoantigen peptides. In some cases, such as for a specific treatment regimen and specific syndromes may benefit from a previous treatment with anti-PD-1, anti-PD-L1, anti-CD40, or anti-CTLA-4 antibodies.

Patients who have recently been administered chemotherapy, targeted small molecule therapy or radiation therapy less than 30 days prior to the treatment regimen may not be able to reconstitute their immune systems to achieve all the benefits from the treatment with the compositions presented herein. Similar results from patients with autoimmune disease that has required systemic treatment in 2 years (i.e., with use of disease-modifying agents, corticosteroids, or immunosuppressive drugs) or a diagnosis of immunodeficiency before the treatment regimen may also not benefit from the treatment regimen. Replacement therapy (e.g., thyroxine, insulin, or physiologic corticosteroid replacement therapy for adrenal or pituitary insufficiency) may not be considered a form of systemic treatment. In such patients, the treatment regimen, dosages may be modified to provide the greatest benefit from the treatment regimen.

Patients with active infections may be given the treatment after the infection is cleared. Patients with sensitivity or allergy to mAbs or IgG may be provided a modified composition to avoid allergic reactions. Patients with a history of allogeneic bone marrow transplantation, a known history of HIV, Hepatitis B, Hepatitis C, uncontrolled intercurrent illness including, but not limited to, ongoing or active infection requiring treatment, symptomatic congestive heart failure, unstable angina pectoris, cardiac arrhythmia may not benefit from treatment with the compositions described herein. Treatment regimens, compositions and dosages may be altered to benefit such patients.

### Example 10—Study Assessments and Schedule of Events

Unless scheduled assessments are adjusted for early or delayed vaccination, assessments will be conducted through Week 60/EOT for Cohort 1 (Treatment Arm C) and through Week 52/EOT for Cohorts 2 and 3 according to the schedule in Table 5.

TABLE 5

| | | Pre- | Pre-vaccination Treatment Period | | | | | |
|---|---|---|---|---|---|---|---|---|
| | Screen¹ | Tx¹ | Day 1/ | | | | | |
| Procedure^{1, 2} | Wk −4 to Wk 0 | | Wk 0 | Wk 2 | Wk 4 | Wk 6 | Wk 8 | Wk 10 |
| Informed consent⁶ | X | | | | | | | |
| Complete medical history | X | | | | | | | |
| including prior cancer | | | | | | | | |
| therapies | | | | | | | | |
| Demographics | X | | | | | | | |
| Symptom-directed | X | | X | X | X | X | X | X |
| physical examination⁷ | | | | | | | | |
| ECOG PS | X | | X | | | | | |
| Vital signs | X | | X | X | X | X | X | X |
| 12-Lead ECG¹⁰ | X | | | | | | | X |
| CT/MRI^{1, 13} | X | | | | | | X | |
| Hematology¹⁵ | X | | X | X | X | X | X | X |
| Chemistry¹⁵ | X | | X | X | X | X | X | X |
| Pregnancy testing¹⁶ | X | | X | X | X | X | X | X |
| Thyroid-stimulating | X | | | | | | | |
| hormone^{15, 17} | | | | | | | | |
| HLA Class I typing | | X | | | | | | |
| Tumor biopsy^{18, 19} | | X | | | | | | X |
| Blood draw for sequencing | | X | | | | | | |
| leukapheresis^{2, 20} | | | | | | | | X |
| 80-mL blood draw² | | X | | | | X | | |
| Vaccination | | | | | | | | |
| administration² | | | | | | | | |
| APX005M administration² | | | | | | | | |
| Ipilimumab | | | | | | | | |
| administration² | | | | | | | | |
| Survival follow-up | | | | | | | | |

| | |
|---|---|
| Nivolumab administration | Nivolumab administered at 240 mg IV Q2W to Wk 52 (Treatment Arms A |
| | and B) or to Wk 60 (Treatment Arm C). Nivolumab administration window is |
| | Q2W ±2 days, with a minimum of 12 days between infusions. |

| AE and SAE collection | SAEs will be collected from signing of ICF through the 90-day | |
|---|---|---|
| | Post-treatment Period unless new treatment is started | |
| | AEs from Day 1/Week 0 through the 30-Day Post-treatment Period visit | |
| Concemitant medication | | Collected from 30 days prior to Day 1/Week 0 throughout the study |
| and procedures | | |

| | | | Post-Vaccination | | | |
|---|---|---|---|---|---|---|
| | Vaccination Treatment Period | | Treatment Period | | | |
| | Cohorts 1-3 | Cohort 1 | Cohorts 1-3 | Cohort 1 | | |
| | Arms A and B | Arm C | Arms A and B | Arm C | Post- | |
| | Wks | Wks | Wks | Wks | Tx⁵ | OS |
| Procedure^{1, 2} | 12-14 | 12-48 | 25-52 (Q2W)³ | 54 and 60⁴ | ±7 d | Check |
| Informed consent⁶ | Refer to the | Refer to the | | | | |
| Complete medical history | following tables | following tables | | | | |
| including prior cancer | depending on | depending on | | | | |
| therapies | Cohort and | Cohort and | | | | |
| Demographics | Treatment Arm | Treatment Arm | | | | |
| Symptom-directed | Cohort 1/Arm A: | Cohort 1/Arm C: | X | X | X | |
| physical examination⁷ | Table 1 | Table 2 | | | | |
| ECOG PS | Cohort 1/Arm B: | | X⁸ | X⁹ | X | |
| Vital signs | Table 1 | | | | | |
| 12-Lead ECG¹⁰ | Cohort 2/Arm A: | | X¹¹ | X¹¹ | X¹² | |
| CT/MRI^{1, 13} | Table 3 | | X¹⁴ | X⁹ | | |
| Hematology¹⁵ | Cohort 2/Arm B: | | X | X | | |
| Chemistry¹⁵ | Table 3 | | X | X | | |
| Pregnancy testing¹⁶ | Cohort 3/Arm A: | | X | X | X | |
| Thyroid-stimulating | Table 4 | | | | | |
| hormone^{15, 17} | Cohort 5/Arm B: | | | | | |
| HLA Class I typing | Table 4 | | | | | |
| Tumor biopsy^{18, 19} | | | | | | |
| Blood draw for sequencing | | | | | | |
| leukapheresis^{2, 20} | | | X²¹ | X⁹ | | |
| 80-mL blood draw² | | | X²² | | | |
| Vaccination | | | | | | |
| administration² | | | | | | |
| APX005M administration² | | | | | | |
| Ipilimumab | | | | | | |
| administration² | | | | | | |
| Survival follow-up | | | | | | X |

| Nivolumab administration | Nivolumab administered at 240 mg IV Q2W to Wk 52 (Treatment Arms A |
|---|---|
| | and B) or to Wk 60 (Treatment Arm C). Nivolumab administration window is |
| | Q2W ±2 days, with a minimum of 12 days between infusions. |
| AE and SAE collection | SAEs will be collected from signing of ICF through the 90-day |
| | Post-treatment Period unless new treatment is started |
| | AEs from Day 1/Week 0 through the 30-Day Post-treatment Period visit |
| Concemitant medication | Collected from 30 days prior to Day 1/Week 0 throughout the study |
| and procedures | |

¹Abbreviations: AE = adverse event; β-hCG = Beta-human chorionic gonadotropin; CT = computed tomography; d = days; ECG = electrocardiogram; ECOG PS = Eastern Cooperative Oncology Group Performance Score; HLA = human leukocyte antigen; ICF = informed consent form; IV = intravenous; MRI = magnetic resonance imaging; OS = overall survival; PD = progressive disease; Q2W = every 2 weeks; RECIST = Response Evaluation Criteria in Solid Tumors; SAE = serious adverse event; Tx = treatment; Wk = weekThe Screening Period and Pre-treatment Period run concurrently over approximately 30 days and may be extended by 15 days (to 45 days total) if a repeat biopsy is required. All Screening Period procedures must be completed to determine eligibility before any Pre-treatment Period procedures can be conducted. Assessments must be completed prior to the first dose of nivolumab at Day 1/Week 0. All study procedures should be performed within ±7 days of the scheduled time, unless specified otherwise.
²At the discretion of the Investigator, patients with PD may begin the Vaccination Treatment Period earlier than 12 weeks, if NEO-PV-01 vaccine is available, while continuing therapy with nivolumab. For these patients, the timing of all treatments and assessments should be adjusted accordingly.
³EOT visit is at Week 52 for Treatment Arms A and B of Cohorts 1, 2, and 3.
⁴EOT visit is at Week 60 for Treatment Arms C of Cohort 1.
⁵Conducted at 30 days (±7 days) and 90 days (±7 days) after last dose of nivolumab.
⁶Informed consent must be obtained before any study-specific procedures are performed.
⁷A symptom-directed physical exam must be conducted at all study visits.
⁸Weeks 36 and 48 only.
⁹Week 60 only.
¹⁰ECGs are permitted at any time during the study, as clinically indicated.
¹¹Conduct ECG at last study visit of the Post-Vaccination Treatment Period.
¹²Only at 90 days (±7 days) follow-up.
¹³CT or MRI of other areas of disease (e.g., neck) should be obtained as clinically indicated. If IV contrast is contraindicated, a non-contrast CT and MRI may be used to evaluate sites of disease where a CT without contrast is not adequate. The imaging modality should remain consistent throughout the study and should be the same modality used at Screening. Clinical assessments must be based on RECIST v1.1.
¹⁴Conduct CT/MRI at Week 36 and Week 52 only.
¹⁵Collect all samples prior to dosing.
¹⁶Women of childbearing potential must have a negative urine or serum β-hCG or urine pregnancy test during Screening (within 7 days of starting treatment). Urine pregnancy testing will be performed at subsequent study visits.
¹⁷Thyroid-stimulating hormone with reflex T4.
¹⁸Additional tumor biopsy to be obtained in patients who show PD and are discontinued from the study.
¹⁹Biopsies may be completed within a 15-day window prior to the study visit and must occur before nivolumab treatment (Week 0, Day 1) and before the first vaccination (Week 12).
²⁰A 15-day window prior to the study visit is allowed for leukapheresis, but the procedure must becompleted prior to any scheduled treatment. If leukapheresis cannot be performed for safety reasons, a total of 180 mL to 200 mL of blood should be drawn as a replacement for this procedure.
²¹Week 52 only.
²²At Week 36 only.

SEQUENCES

| SEQ | |
|---|---|
| ID | |
| NO. | Sequence |
| 1 | GFSFSSTYVC |
| 2 | CIYTGDGTNYSASWAK |
| 3 | PDITYGFAINF |
| 4 | QASQSISSRLA |
| 5 | RASTLAS |
| 6 | QCTGYGISWP |
| 7 | QVQLVESGGGVVQPGRSLRLSCAASGFSFSSTYVCWVRQAPGKGL |
| | EWIACIYTGDGTNYSASWAKGRFTISKDSSKNTVYLQMNSLRAED |
| | TAVYFCARPDITYGFAINFWGPGTLVTVSS |
| 8 | MDMRVPAQLLGLLLLWLRGARCDIQMTQSPSSLSASVGDRVTIKC |
| | QASQSISSRLAWYQQKPGKPPKWYRASTLASGVPSRFSGSGSGTD |
| | FTLTISSLQPEDVATYYCQCTGYGISWPIGGGTKVEIK |
| 9 | METGLRGLLLVAVLKGVQCQSLEESGGDLVKPGASLTLTCTASGF |
| | SFSSTYVCWVRQAPGKGLEWIACIYTGDGTNYSASWAKGRFTISK |
| | PSSTTVTLQMTSLTPADTATYFCARPDITYGFAINFWGPGTLVTV |
| | SS |
| 10 | MDTRAPTQLLGLLLLWLPGARSADIVMTQTPSSASEPVGGTVTIKC |
| | QASQSISSRLAWYQQKPGQPPKWYRASTLASGVPSRFKGSGSGTE |
| | FTLTISDLECADAATYYCQCTGYGISWPIGGGTEVVVK |

1. A method of treating or preventing a neoplasia in a human subject in need thereof comprising administering to a subject in need thereof:
(a) a first component comprising (i) a peptide comprising a neoepitope of a protein expressed by the human subject, (ii) a polynucleotide encoding the peptide, (iii) one or more APCs comprising the peptide or the polynucleotide encoding the peptide, or (iv) T cells comprising a T cell receptor (TCR) specific for the neoepitope in complex with an HLA protein expressed by the human subject; and
(b) a second component comprising at least two inhibitors, wherein the at least two inhibitors comprise: (i) nivolumab and an anti-CD40 agonist antibody, or (ii) nivolumab and ipilimumab, or (iii) ipilimumab and an anti-CD40 agonist antibody.

2. The method of claim 1, wherein the anti-CD40 agonist antibody comprises a heavy chain complement determining region 1 (HCDR1) of SEQ ID NO: 1, a HCDR2 of SEQ ID NO: 2, and a HCDR3 of SEQ ID NO: 3; and a light chain CDR1 (LCDR1) of SEQ ID NO: 4, a LCDR2 of SEQ ID NO: 5 and an LCDR3 of SEQ ID NO: 6.

3. The method of claim 2, wherein the anti-CD40 agonist antibody comprises a heavy chain variable sequence (V_{H}) with at least 80% sequence identity to SEQ ID NO: 7, and a light chain variable sequence (V_{L}) with at least 80% sequence identity to SEQ ID NO: 8.

4. The method of claim 2, wherein the anti-CD40 agonist antibody comprises a heavy chain sequence with at least 70% sequence identity to SEQ ID NO: 9, and a light chain sequence with at least 70% sequence identity to SEQ ID NO: 10.

5. (canceled)

6. (canceled)

7. (canceled)

8. The method of claim 1, wherein the first component further comprises an adjuvant.

9. The method of claim 8, wherein the adjuvant is poly-ICLC.

10. The method of claim 1, wherein the first component comprises T cells comprising a first TCR specific for a first neoepitope in complex with a first HLA protein and a second TCR specific for a second neoepitope in complex with a second HLA protein.

11-16. (canceled)

17. The method of claim 1, wherein the neoplasia is selected from the group consisting of Non-Hodgkin's Lymphoma (NHL), clear cell Renal Cell Carcinoma (ccRCC), melanoma, sarcoma, leukemia, bladder cancer, colon cancer, brain cancer, breast cancer, head and neck cancer, endometrial cancer, lung cancer, ovarian cancer, uterine cancer, pancreatic cancer, stomach cancer, esophageal cancer, thyroid cancer, prostate cancer, a cancer associated with microsatellite instability (MSI), a mismatch repair deficient (dMMR) cancer, unresectable melanoma, metastatic melanoma, metastatic non-small cell lung cancer, advanced renal cell carcinoma, classical Hodgkin lymphoma, squamous cell carcinoma of the head and neck, urothelial carcinoma, microsatellite instability-high (MSI-H) metastatic colorectal cancer, dMMR metastatic colorectal cancer, hepatocellular carcinoma and combinations thereof.

18-20. (canceled)

21. The method of any of claim 1, wherein the second component is administered before the first component.

22. (canceled)

23. (canceled)

24. The method of any of claim 1, wherein administration of nivolumab is initiated before initiation of administration of the first component.

25. The method of claim 1, wherein administration of nivolumab is initiated before initiation of administration of the anti-CD40 agonist antibody.

26-31. (canceled)

32. The method of claim 1, wherein administration of the first component is in a prime boost dosing regimen.

33. The method of claim 0, wherein administration of the first component is at weeks 1, 2, 3 or 4 as a prime; and wherein administration of the first component is at weeks 19, 20, 21, 22, 23 or 24 or months 2, 3, 4 or 5 as a boost.

34-37. (canceled)

38. The method of claim 1, wherein nivolumab is administered at a dose of from 200-260 mg.

39. The method of claim 2, wherein the anti-CD40 agonist antibody is administered at a dose of from 0.05-0.2 mg/kg.

40. The method of claim 2, wherein the anti-CD40 agonist antibody is administered at a dose of from 0.5-2.0 mg/kg.

41-53. (canceled)

54. A method of treating or preventing cancer in a human subject in need thereof that has been treated with nivolumab comprising administering to the subject:
(a) T cells comprising a T cell receptor (TCR) specific for the neoepitope in complex with an HLA protein; and
(b) an anti-CD40 agonist antibody that is APX005M at a dose of from 0.05-2.0 mg/kg, wherein the anti-CD40 agonist antibody comprises a heavy chain complement determining region 1 (HCDR1) of SEQ ID NO: 1, a HCDR2 of SEQ ID NO: 2, and a HCDR3 of SEQ ID NO: 3; and a light chain CDR1 (LCDR1) of SEQ ID NO: 4, a LCDR2 of SEQ ID NO: 5 and an LCDR3 of SEQ ID NO: 6.

55. A method of treating or preventing cancer in a human subject in need thereof that has been treated with nivolumab comprising administering to the subject:
(a) T cells comprising a T cell receptor (TCR) specific for the neoepitope in complex with an HLA protein; and
(b) ipilimumab at a dose of from 0.5-1.5 mg/kg.

56-60. (canceled)

61. A method of treating or preventing cancer in a human subject in need thereof comprising administering to the subject:
(a) nivolumab at a dose of less than 3.0 mg/kg; and
(b) an anti-CD40 agonist antibody at a dose of less than 1.0 mg/kg, wherein the anti-CD40 agonist antibody comprises a heavy chain complement determining region 1 (HCDR1) of SEQ ID NO: 1, a HCDR2 of SEQ ID NO: 2, and a HCDR3 of SEQ ID NO: 3; and a light chain CDR1 (LCDR1) of SEQ ID NO: 4, a LCDR2 of SEQ ID NO: 5 and an LCDR3 of SEQ ID NO: 6.

62. The method of claim 61, wherein the nivolumab is administered at a dose of less than 1.0 mg/kg.

63. The method of claim 61, wherein the anti-CD40 agonist antibody is administered at a dose of less than 0.1 mg/kg.

64. The method of claim 62, wherein the anti-CD40 agonist antibody is administered at a dose of less than 0.1 mg/kg.

65. (canceled)

66. A method of treating or preventing cancer in a human subject in need thereof that has been treated with nivolumab comprising administering to the subject:
(a) nivolumab at a dose of less than 3.0 mg/kg; and
(b) ipilimumab at a dose of less than 1.0 mg/kg.

67. The method of claim 66, wherein the nivolumab is administered at a dose of less than 1.0 mg/kg.

68-71. (canceled)
